SEQUENCE LISTING (24 sequences)

SEQ ID NO: 1 - protein, 3163 aa, Yersinia pestis
MDNLRFSSAPTADSIDASIAQHYPDCEPVAVIGYACHFPESPDGETFWQNLLEGRECSRRFTREELLAVGLDAAIIDDPHYVNIGTVLDNADCFDATLFGYSRQEAESMDPQQRLFLQAVWHALEHAGYAPGAVPHKTGVFASSRMSTYPGREALNVTEVAQVKGLQSLMGNDKDYIATRAAYKLNLHGPALSVQTACSSSLVAVHLACESLRAGESDMAVAGGVALSFPQQAGYRYQPGMIFSPDGHCRPFDASAEGTWAGNGLGCVVLRRLRDALLSGDPIISVILSSAVNNDGNRKVGYTAPSVAGQQAVIEEALMLAAIDDRQVGYIETHGTGTPLGDAIEIEALRNVYAPRPQDQRCALGSVKSNMGHLDTAAGIAGLLKTVLAVSRGQIPPLLNFHTPNPALKLEESPFTIPVSAQAWQDEMRYAGVSSFGIGGTNCHMIVASLPDALNARLPNTDSGRKSTALLLSAASDSALRRLATDYAGALRENADASSLAFTALHARRLDLPFRLAAPLNRETAEALSAWAGEKSGALVYSGHGASGKQVWLFTGQGSHWRTMGQTMYQHSTAFADTLDRCFSACSEMLTPSLREAMFNPDSAQLDNMAWAQPAIVAFEIAMAAHWRAEGLKPDFAIGHSVGEFAAAVVCGHYTIEQVMPLVCRRGALMQQCASGAMVAVFADEDTLMPLARQFELDLAANNGTQHTVFSGPEARLAVFCATLSQHDINYRRLSVTGAAHSALLEPILDRFQDACAGLHAEPGQIPIISTLTADVIDESTLNQADYWRRHMRQPVRFIQSIQVAHQLGARVFLEMGPDAQLVACGQREYRDNAYWIASARRNKEASDVLNQALLQLYAAGVALPWADLLAGDGQRIAAPCYPFDTERYWKERVSPACEPADAALSAGLEVASRAATALDLPRLEALKQCATRLHAIYVDQLVQRCTGDAIENGVDAMTIMRRGRLLPRYQQLLQRLLNNCVVDGDYRCTDGRYVRARPIEHQQRESLLTELAGYCEGFQAIPDTIARAGDRLYEMMSGAEEPVAIIFPQSASDGVEVLYQEFSFGRYFNQIAAGVLRGIVQTRQPRQPLRILEVGGGTGGTTAWLLPELNGVPALEYHFTDISALFTRRAQQKFADYDFVKYSELDLEKEAQSQGFQAQSYDLIVAANVIHATRHIGRTLDNLRPLLKPGGRLLMREITQPMRLFDFVFGPLVLPLQDLDAREGELFLTTAQWQQQCRHAGFSKVAWLPQDGSPTAGMSEHIILATLPGQAVSAVTFTAPSEPVLGQALTDNGDYLADWSDCAGQPERFNARWQEAWRLLSQRHGDALPVEPPPVAAPEWLGKVRLSWQNEAFSRGQMRVEARHPTGEWLPLSPAAPLPAPQTHYQWRWTPLNVASIDHPLTFSFSAGTLARSDELAQYGIIHDPHASSRLMIVEESEDTLALAEKVIAALTASAAGLIVVTRRAWRVEENEALSASHHALWALLRVAANEQPERLLAAIDLAENTPWETLHQGLSAVSLSQRWLAARGDTLWLPSLAPNTGCAAELPANVFTGDSRWHLVTGAFGGLGRLAVNWLREKGARRIALLAPRVDESWLRDVEGGQTRVCRCDVGDAGQLATVLDDLAANGGIAGAIHAAGVLADAPLQELDDHQLAAVFAVKAQAASQLLQTLRNHDGRYLILYSSAAATLGAPGQSAHALACGYLDGLAQQFSTLDAPKTLSVAWGAWGESGRAATPEMLATLASRGMGALSDAEGCWHLEQAVMRGAPWRLAMRVFTDKMPPLQQALFNISATEKAATPVIPPADDNAFNGSLSDETAVMAWLKKRIAVQLRLSDPASLHPNQDLLQLGMDSLLFLELSSDIQHYLGVRINAERAWQDLSPHGLTQLICSKPEATPAASQPEVLRHDADERYAPFPLTPIQHAYWLGRTHLIGYGGVACHVLFEWDKRHDEFDLAILEKAWNQLIARHDMLRMVVDADGQQRILATTPEYHIPRDDLRALSPEEQRIALEKRRHELSYRVLPADQWPLFELVVSEIDDCHYRLHMNLDLLQFDVQSFKVMMDDLAQVWRGETLAPLAITFRDYVMAEQARRQTSAWHDAWDYWQEKLPQLPLAPELPVVETPPETPHFTTFKSTIGKTEWQAVKQRWQQQGVTPSAALLTLFAATLERWSRTTTFTLNLTFFNRQPIHPQINQLIGDFTSVTLVDFNFSAPVTLQEQMQQTQQRLWQNMAHSEMNGVEVIRELGRLRGSQRQPLMPVVFTSMLGMTLEGMTIDQAMSHLFGEPCYVFTQTPQVWLDHQVMESDGELMFSWYCMDNVLEPGAAEAMFNDYCAILQAVIAAPESLKTLASGIAGHIPRRRWPLNAQADYDLRDIEQATLEYPGIRQARAEITEQGALTLDIVMADDPSPSAAMPDEHELTQLALPLPEQAQLDELEATWRWLEARALQGIAATLNRHGLFTTPEIAHRFSAIVQALSAQASHQRLLRQWLQCLTEREWLIREGESWRCRIPLSEIPEPQEACPQSQWSQALAQYLETCIARHDALFSGQCSPLELLFNEQHRVTDALYRDNPASACLNRYTAQIAALCSAERILEVGAGTAATTAPVLKATRNTRQSYHFTDVSAQFLNDARARFHDESQVSYALFDINQPLDFTAHPEAGYDLIVAVNVLHDASHVVQTLRRLKLLLKAGGRLLIVEATERNSVFQLASVGFIEGLSGYRDFRRRDEKPMLTRSAWQEVLVQAGFANELAWPAQESSPLRQHLLVARSPGVNRPDKKAVSRYLQQRFGTGLPILQIRQREALFTPLHAPSDAPTEPAKPTPVAGGNPALEKQVAELWQSLLSRPVARHHDFFELGGDSLMATRMVAQLNRRGIARANLQDLFSHSTLSDFCAHLQAATSGEDNPIPLCQGDGEETLFVFHASDGDISAWLPLASALNRRVFGLQAKSPQRFATLDQMIDEYVGCIRRQQPHGPYVLAGWSYGAFLAAGAAQRLYAKGEQVRMVLIDPVCRQDFCCENRAALLRLLAEGQTPLALPEHFDQQTPDSQLADFISLAKTAGMVSQNLTLQAAETWLDNIAHLLRLLTEHTPGESVPVPCLMVYAAGRPARWTPAETEWQGWINNADDAVIEASHWQIMMEAPHVQACAQHITRWLCATSTQPENTL

SEQ ID NO: 2 - protein, 2035 aa, Yersinia pestis
MISGAPSQDSLLPDNRHAADYQQLRERLIQELNLTPQQLHEESNLIQAGLDSIRLMRWLHWFRKNGYRLTLRELYAAPTLAAWNQLMLSRSPENAEEETPPDESSWPNMTESTPFPLTPVQHAYLTGRMPGQTLGGVGCHLYQEFEGHCLTASQLEQAITTLLQRHPMLHIAFRPDGQQVWLPQPYWNGVTVHDLRHNDAESRQAYLDALRQRLSHRLLRVEIGETFDFQLTLLPDNRHRLHVNIDLLIMDASSFTLFFDELNALLAGESLPAIDTRYDFRSYLLHQQKINQPLRDDARAYWLAKASTLPPAPVLPLACEPATLREVRNTRRRMIVPATRWHAFSNRAGEYGVTPTMALATCFSAVLARWGGLTRLLLNITLFDRQPLHPAVGAMLADFTNILLLDTACDGDTVSNLARKNQLTFTEDWEHRHWSGVELLRELKRQQRYPHGAPVVFTSNLGRSLYSSRAESPLGEPEWGISQTPQVWIDHLAFEHHGEVWLQWDSNDALFPPALVETLFDAYCQLINQLCDDESAWQKPFADMMPASQRAIRERVNATGAPIPEGLLHEGIFRIALQQPQALAVTDMRYQWNYHELTDYARRCAGRLIECGVQPGDNVAITMSKGAGQLVAVLAVLLAGAVYVPVSLDQPAARREKIYADASVRLVLICQHDASAGSDDIPVLAWQQAIEAEPIANPVVRAPTQPAYIIYTSGSTGTPKGVVISHRGALNTCCDINTRYQVGPHDRVLALSALHFDLSVYDIFGVLRAGGALVMVMENQRRDPHAWCELIQRHQVTLWNSVPALFDMLLTWCEGFADATPENLRAVMLSGDWIGLDLPARYRAFRPQGQFIAMGGATEASIWSNACEIHDVPAHWRSIPYGFPLTNQRYRVVDEQGRDCPDWVPGELWIGGIGVAEGYFNDPLRSEQQFLTLPDERWYRTGDLGCYWPDGTIEFLGRRDKQVKVGGYRIELGEIESALSQLAGVKQATVLAIGEKEKTLAAYVVPQGEAFCVTDHRNPALPQAWHTLAGTLPCCAISPEISAEQVADFLQHRLLKLKPGHTAGADPLPLMNSLAIQPRWQAVVERWLAFLVTQRRLKPAAEGYQVCAGEEREDEHPHFSGHDLTLSQILRGARNELSLLNDAQWSPESLAFNHPASAPYIQELATICQQLAQRLQRPVRLLEVGTRTGRAAESLLAQLNAGQIEYVGLEQSQEMLLSARQRLAPWPGARLSLWNADTLAAHAHSADIIWLNNALHRLLPEDPGLLATLQQLAVPGALLYVMEFRQLTPSALLSTLLLTNGQPEALLHNSADWAALFSAAAFNCQHGDEVAGLQRFLVQCPDRQVRRDPRQLQAALAGRLPGWMVPQRIVFLDALPLTANGKIDYQALKRRHTPEAENPAEADLPQGDIEKQVAALWQQLLSTGNVTRETDFFQQGGDSLLATRLTGQLHQAGYEAQLSDLFNHPRLADFAATLRKTDVPVEQPFVHSPEDRYQPFALTDVQQAYLVGRQPGFALGGVGSHFFVEFEIADLDLTRLETVWNRLIARHDMLRAIVRDGQQQVLEQTPPWVIPAHTLHTPEEALRVREKLAHQVLNPEVWPVFDLQVGYVDGMPARLWLCLDNLLLDGLSMQILLAELEHGYRYPQQLLPPLPVTFRDYLQQPSLQSPNPDSLAWWQAQLDDIPPAPALPLRCLPQEVETPRFARLNGALDSTRWHRLKKRAADAHLTPSAVLLSVWSTVLSAWSAQPEFTLNLTLFDRRPLHPQINQILGDFTSLMLLSWHPGESWLHSAQSLQQRLSQNLNHRDVSAIRVMRQLAQRQNVPAVPMPVVFTSALGFEQDNFLARRNLLKPVWGISQTPQVWLDHQIYESEGELRFNWDFVAALFPAGQVERQFEQYCALLNRMAEDESGWQLPLAALVPPVKHAGQCAERSPRVCPEHSQPHIAADESTVSLICDAFREVVGESVTPAENFFEAGATSLNLVQLHVLLQRHEFSTLTLLDLFTHPSPAALADYLAGVATVEKTKRPRPVRRRQRRI

SEQ ID NO: 3 - protein, 525 aa, Yersinia pestis
MNSSFESLIEQYPLPIAEQLRHWAARYASRIAVVDAKGSLTYSALDAQVDELAAGLSSLGLRSGEHVIVQLPNDNAFVTLLFALLRLGVIPVLAMPSQRALDIDALIELAQPVAYVIHGENHAELARQMAHKHACLRHVLVAGETVSDDFTPLFSLHGERQAWPQPDVSATALLLLSGGTTGTPKLIPRRHADYSYNFSASAELCGISQQSVYLAVLPVAHNFPLACPGILGTLACGGKVVLTDSASCDEVMPLIAQERVTHVALVPALAQLWVQAREWEDSDLSSLRVIQAGGARLDPTLAEQVIATFDCTLQQVFGMAEGLLCFTRLDDPHATILHSQGRPLSPLDEIRIVDQDENDVAPGETGQLLTRGPYTISGYYRAPAHNAQAFTAQGFYRTGDNVRLDEVGNLHVEGRIKEQINRAGEKIAAAEVESALLRLAEVQDCAVVAAPDTLLGERICAFIIAQQVPTDYQQLRQQLTRMGLSAWKIPDQIEFLDHWPLTAVGKIDKKRLTALAVDRYRHSAQ

SEQ ID NO: 4 - protein, 386 aa, Yersinia pestis
MCATHYALALRNLNATGEHVMMPSASPKQRVLIVGAKFGEMYLNAFMQPPEGLELVGLLAQGSARSRELAHAFGIPLYTSPEQITRMPDIACIVVRSTVAGGTGTQLARHFLTRGVHVIQEHPLHPDDISSLQTLAQEQGCCYWVNTFYPHTRAGRTWLRDAQQLRRCLAKTPPVVHATTSRQLLYSTLDLLLLALGVDAAAVECDVVGSFSDFHCLRLFWPEGEACLLLQRYLDPDDPDMHSLIMHRLLLGWPEGHLSLEASYGPVIWSSSLFVADHQENAHSLYRRPEILRDLPGLTRSAAPLSWRDCCETVGPEGVSWLLHQLRSHLAGEHPPAACQSVHQIALSRLWQQILRKTGNAEIRRLTPPHHDRLAGFYNDDDKEAL

SEQ ID NO: 5 - protein, 242 aa, Yersinia pestis
MSVLNIPLLPPFITHASLQPLDGHPCVDRLDVRFDAGHFTPSLFSTLDIPCPAHLERSVNKRRAEYLVSRYGLQQALASLGIARFVLGNDENRAPIWPAGIAGSLSHTHQQVCALLTRDDNLLLGIDCEQMMTLDTASEMQSMLINQRERERLEQCSLPFNHALTVVFSLKESLYKALYPRLKQFMDFSAAEVMECYPDMQQVSLRLTQTFSAEMVAGRVFTGHAVLQPDRVMTWVVEPQPR

SEQ ID NO: 6 - protein, 434 aa, Yersinia pestis
MKISEFLHLALPEEQWLPTISGVLRQFAEEECYVYERPPCWYLGKGCQARLHINADGTQATFIDDAGEQKWAVDSIADCARRFMAHPQVKGRRVYGQVGFNFAAHARGIAFNAGEWPLLTLTVPREELIFEKGNVTVYADSADGCRRLCEWVKEASTTTQNAPLAVDTALNGEAYKQQVARAVAEIRRGEYVKVIVSRAIPLPSRIDMPATLLYGRQANTPVRSFMFRQEGREALGFSPELVMSVTGNKVVTEPLAGTRDRMGNPEHNKAKEAELLHDSKEVLEHILSVKEAIAELEAVCLPGSVVVEDLMSVRQRGSVQHLGSGVSGQLAENKDAWDAFTVLFPSITASGIPKNAALNAIMQIEKTPRELYSGAILLLDDTRFDAALVLRSVFQDSQRCWIQAGAGIIAQSTPERELTETREKLASIAPYLMV

SEQ ID NO: 7 - protein, 267 aa, Yersinia pestis
MTQSAMCIPLWPARNGNTAHLVMCPFAGGSSSAFRHWQAEQLTDCALSLVTWPGRDRLRHLEPLRSITQLAALLANELEASVSPDTPLLLAGHSMGAQVAFETCRLLEQRGLAPQGLIISGCHAPHLHSERQLSHRDDADFIAELIDIGGCSPELRENQELMSLFLPLLRADFYATESYHYDSPDVCPPLRTPALLLCGSHDREASWQQVDAWRQWLSHVTGPVVIDGDHFYPIQQARSFFTQIVRHFPHAFSAMTALQKQPSTSER

SEQ ID NO: 8 - protein, 448 aa, Yersinia pestis
MVMSQKTLFTKSALAVAVALISTQAWSAGFQLNEFSSSGLGRAYSGEGAIADDAGNVSRNPALITMFDRPTFSAGAVYIDPDVNISGTSPSGRSLKADNIAPTAWVPNMHFVAPINDQFGWGASITSNYGLATEFNDTYAGGSVGGTTDLETMNLNLSGAYRLNNAWSFGLGFNAVYVRAKIERFAGDLGQLVAGQIMQSPAGQTQQGQALAATANGIDSNTKIAHLNGNQWGFGWNAGILYELDKNNRYALTYRSEVKIDFKGNYSSDLNRAFNNYGLPIPTATGGRTQSGYLTLNLPEMWEVSGYNRVDPQWAIHYSLAYTSWSQFQQLKATSTSGDTLFQKHEGFKDAYRIALGTTYYYDDNWTFRTGIAFDDSPVPAQNRSISIPDQDRFWLSAGTTYAFNKDASVDVGVSYMHGQSVKINEGPYQFESEGKAWLFGTNFNYAF

SEQ ID NO: 9 - DNA, 30 bp, artificial sequence
  primer
aatcatatgaaaatcagtgaatttctacac

SEQ ID NO: 10 - DNA, 31 bp, artificial sequence
  primer
aatcctcgagctactacaccattaaataggg

SEQ ID NO: 11 - DNA, 27 bp, artificial sequence
  primer
aatcatatggtcatgagccagaaaacc

SEQ ID NO: 12 - DNA, 30 bp, artificial sequence
  primer
aatctcgagtcagaacgcgtagttaaagtt

SEQ ID NO: 13 - DNA, 21 bp, artificial sequence
  primer
atggtcatgagccagaaaacc

SEQ ID NO: 14 - DNA, 21 bp, artificial sequence
  primer
tcagaacgcgtagttaaagtt

SEQ ID NO: 15 - DNA, 29 bp, artificial sequence
  primer
attcaattggtgtaggctggagctgcttc

SEQ ID NO: 16 - DNA, 29 bp, artificial sequence
  primer
attggtaccatgggaattagccatggtcc

SEQ ID NO: 17 - DNA, 38 bp, artificial sequence
  primer
tggggcgcttctattacccgtaactatggtctggctac

SEQ ID NO: 18 - DNA, 38 bp, artificial sequence
  primer
gtagccagaccatagttacgggtaatagaagcgcccca

SEQ ID NO: 19 - DNA, 29 bp, artificial sequence
  primer
tgttcggaacccattccgtaggcgcgatg

SEQ ID NO: 20 - DNA, 29 bp, artificial sequence
  primer
catcgcgcctacggaatgggttccgaaca

SEQ ID NO: 21 - DNA, 30 bp, artificial sequence
  primer
attcatatggctgacattctgctgctcgat

SEQ ID NO: 22 - DNA, 30 bp, artificial sequence
  primer
attctcgagttaccctcgtgccgccagtgc

SEQ ID NO: 23 - DNA, 31 bp, artificial sequence
  primer
attgaattcgcaaacacaaaaaccgactctc

SEQ ID NO: 24 - DNA, 32 bp, artificial sequence
  primer
attgcggccgctcagaaagtctcctgtgcatg

(19) United States
(12) Patent Application Publication
(10) Pub. No.: US 2018/0065940 A1
(43) Pub. Date: Mar. 8, 2018

(54) METAL-BINDING COMPOUNDS, HETEROLOGOUS PRODUCTION AND USES THEREOF

(71) Applicant: The Research Foundation for The State University of New York, Buffalo, NY (US)

(72) Inventors: Blaine PFEIFER, Buffalo, NY (US); Mahmoud AHMADI, Amherst, NY (US)

(21) Appl. No.: 15/559,604

(22) PCT Filed: Mar. 21, 2016
(86) PCT No.: PCT/US16/23434

§ 371 Date: Sep. 19, 2017

Related U.S. Application Data
(60) Provisional application No. 62/136,416, filed on Mar. 20, 2015.

(51) Int. Cl. C07D 277/08; C12P 17/16; C12P 21/02; C02F 1/68; C02F 3/34

(52) U.S. Cl. CPC C07D 277/08; C12P 17/167; C02F 2101/203; C02F 1/683; C02F 3/346; C12P 21/02

(57) ABSTRACT

Provided are Ybt analogs and derivatives thereof and compositions comprising Ybt and Ybt analogs and derivatives thereof. Methods of making Ybt and Ybt analogs and methods of using Ybt and Ybt analogs are also provided. The methods use microbes modified to produce Ybt and Ybt analogs. The compounds and compositions can be used to remove metals from metal containing samples.

### CROSS-REFERENCE TO RELATED APPLICATIONS

This application claims priority to U.S. provisional patent application No. 62/136,416, filed Mar. 20, 2015, the disclosure of which is incorporated herein by reference.

### STATEMENT REGARDING FEDERALLY SPONSORED RESEARCH

This invention was made with government support under contract no. 1550378 awarded by the National Science Foundation. The government has certain rights in the invention.

### BACKGROUND OF THE DISCLOSURE

Precious metals such as gold, silver, palladium, and platinum have been sought after for investment, adornment, and industrial use for centuries. However, ever-growing applications in catalysis, electronics, and electroplating have given rise to gradual loss through waste effluents in the automotive, medical, computer, and solar panel industries. Effluents in these industries can contain low metal concentrations, in the range of 0.1 ppm (ppm—parts per million), which makes recovery a technical challenge. It is estimated that at least $2 billion of precious metals are lost globally in these industries annually.

Various techniques have been developed to capture and recycle precious metals from waste streams. Currently, the most effective recovery methods utilize functionalized absorbents known as metal scavengers. Metal scavengers provide recovery down to concentrations between 0.1 to 1 ppm. However, current scavengers are not efficient or economically feasible for metal concentrations less than 0.1 ppm. Furthermore, the bond between the resin and precious metals can be very strong and non-specific, resulting in irreversible binding to multiple less-desirable metals. These factors make recovery of precious metals challenging, offering no opportunity for simple recovery from the scavengers in a way that is economical for small and moderate enterprises.

### SUMMARY OF THE DISCLOSURE

The present disclosure, in various examples, provides compounds comprising the following structure:

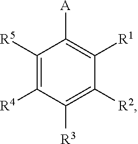

where R¹ is OH or NH₂, and R², R³, R⁴, and R⁵ are each independently H, OH, NH₂, SH, I, Br, Cl, F, alkyl (e.g., C₁-C₆ alkyl), or one or more adjacent combinations of R², R³, R⁴, and/or R⁵ that together form one or more fused rings, and A is selected from the group consisting of:

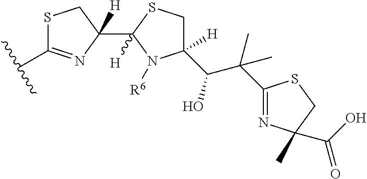

and

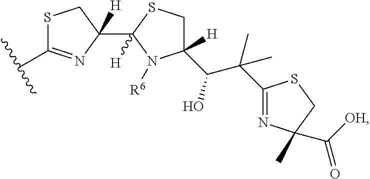

where R⁶ is H or CH₃. In an example, R⁶ is H. In another example, the compound is selected from the group consisting of:

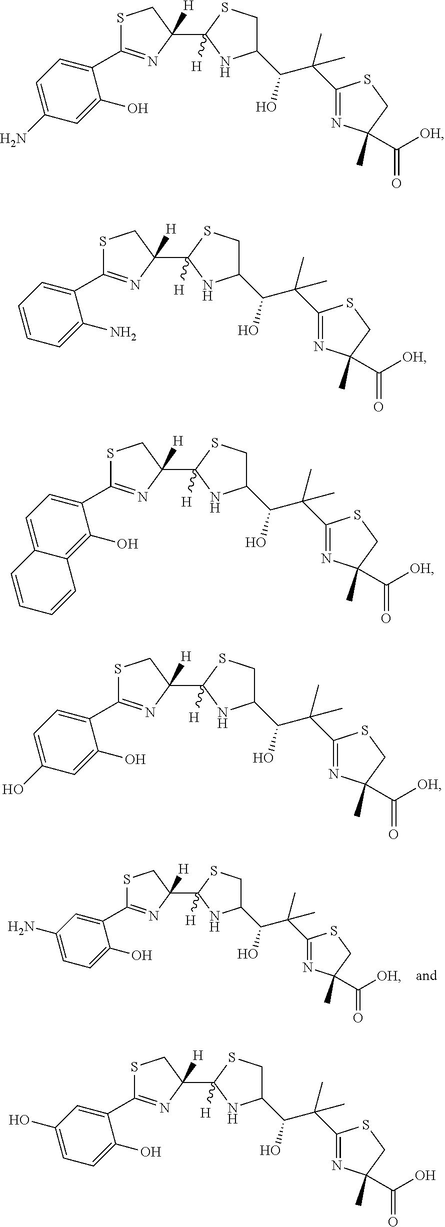

The present disclosure also provides methods of making one or more compounds of the present disclosure. For example, a method comprises contacting one or more microbes (e.g. Escherichia coli cells) modified to produce one or more compounds of the present disclosure with one or more compounds having the following structure:

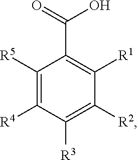

where R¹ is OH or NH₂, and R², R³, R⁴, and R⁵ are each independently H, OH, NH₂, SH, I, Br, Cl, F, alkyl (C₁-C₆ alkyl), or one or more adjacent combinations of R², R³, R⁴, and/or R⁵ that together form one or more fused rings, where a (e.g., for a period of time sufficient) to produce the one or more compounds of the present disclosure. The method may further comprising isolating the one or more compounds of the present disclosure.

The present disclosure also provides methods of removing one or more metals from a metal-containing sample. For example, a method comprises a) contacting the metal-containing sample with the one or more compounds of the present disclosure, where a complex between the one or more metals and the one or more compounds is formed and b) isolating the complex of a) from the sample. The method may further comprise reversing the complex (e.g., the isolated complex) between the one or more metals and one or more compounds of the present disclosure to provide one or more metal-free compounds of the present disclosure and uncomplexed one or more metals. The method may further comprise repeating step a) on the metal-containing sample or a second metal-containing sample using the metal-free compounds of the present disclosure and isolating the complex between the one or more metals from the metal-containing sample and metal-free compounds or the complex between the one or metals of the second metal-containing sample and metal-free compounds. In any of the steps of the methods described herein the compounds of the present disclosure are attached to a polymeric bead, polymeric resin, non-polymeric resin, activated carbon, or inorganic material. The polymeric bead may comprise a non-ionic crosslinked polymer or a hydrophobic resin. In an example the metal-containing sample is selected from the group consisting of water, ocean water, freshwater, tap water, pool water, wastewater, brine, rust-containing surface, mine tailings/waste, semiconductor effluent, jewelry and plating effluents, solar panel effluents, wastewater treatment sludge or precipitants, soil, electronic waste, coal, and fish farm samples. In an example the metal is selected from the group consisting of iron, copper, gold, silver, palladium, platinum, lithium, aluminum, bismuth, gallium, germanium, indium, rhodium, selenium, silicon, nickel, tellurium, zinc, manganese, arsenic, lead, mercury, cerium, scandium, yttrium, lanthanum, praseodymium, neodymium, promethium, samarium, europium, gadolinium, terbium, dysprosium, holmium, erbium, thulium, ytterbium, lutetium, and combinations thereof.

The present disclosure also provides a composition comprising modified microbes comprising a compound of the present disclosure. In an example, the composition comprises modified microbes comprising a compound of the present disclosure, where the modified microbes are modified Escherichia coli, Bacillus subtilis, Streptomyces coelicolor, Streptomyces lividans, Streptomyces venezuelae, Saccharomyces cerevisiae, Pichia pastoris, Aspergillus niger, Aspergillus nidulans, Pseudomonas aeruginosa, or a combination thereof.

### BRIEF DESCRIPTION OF THE FIGURES

FIG. 1. Example of modular biosynthesis of yersiniabactin (Ybt). A salicylate starter unit is activated by YbtE, via adenylation (A), prior to transfer to the first nonribosomal peptide synthetase module of HMWP2 which subsequently introduces and cyclizes two cysteine groups. Upon transfer to HMWP1, a polyketide synthase model introduces a malonyl-CoA unit whose carbonyl group is reduced and methylated in NADPH- and SAM-dependent steps via the activity of dedicated ketoreductase (KR) and methyltransferase (MT) domains. The second module of HMWP1 introduces and cyclizes a third cysteine unit. A terminal thioesterase (TE) domain releases the mixed nonribosomal peptide-polyketide chain from HMWP1 prior to a final reduction step by YbtU. ArCP: aryl carrier protein; Cy: cyclization domain; PCP: peptidyl carrier protein; KS: ketosynthase; AT: acyltransferase; Red: reductase.

FIG. 2. Example of precursor-directed biosynthesis of a Ybt analog. A) Comparison of the native salicylate starter unit and Ybt compound to an analog resulting from feeding of an alternative anthranilate starter unit (note: in this system, the irp9 gene is not included). B) Production comparison of native and altered Ybt.

FIG. 3. Ybt (analog 3) and other analogs generated by precursor-directed biosynthesis. To the left of each numbered analog appears the precursor used in its biosynthesis.

FIG. 4. Examples of Ybt analogs that can be generated by precursor-directed biosynthesis.

FIG. 5. Combination mixtures tested at pH 7.5. Al, Cu, Fe, Zn each with 50 ppb. Pt, Pd, Au, Ag each with 50 ppb.

FIG. 6. Precious metal mixtures tested at pH 8.0. Li, Pt, Pd, Au, Ag each with 50 ppb.

FIG. 7. Example of analog 2 binding Au. A) Plot of intensity vs. time. B) Plot of intensity vs. m/z, amu.

FIG. 8. Example of analog 2 binding Pt. A) Plot of intensity vs. time. B) Plot of intensity vs. m/z, amu.

FIG. 9. Example of analog 2 binding Ag. A) Plot of intensity vs. time. B) Plot of intensity vs. m/z, amu.

FIG. 10. Example of Ybt iron binding and application. A) LC/MS (left) and MS/MS (right) analysis of Ybt-Fe³⁺ complexes. B) Removal ability of Ybt-containing crude extract compared to negative (water, methanol, BAP1/pBP198/pCDFDuet-1-irp9) and positive (commercial product [Rust-Oleum]) controls (left). Visual indication of rust removal from U-shaped steel tube pieces before (i), during (ii), and after (iii) Ybt treatment (right).

FIG. 11. Example of Ybt and analog application to copper removal and wastewater treatment. A) LC/MS (top) and MS/MS (bottom) analysis of Ybt-Cu2+ complexes. B) Visual comparison of XAD and Ybt-XAD used to chelate copper. The XAD-bound Ybt and complexed copper is removed upon washing with methanol for 15 minutes. C) Comparison between non-modified XAD particles and Ybt-modified XAD in a step-wise copper removal process. D) Wastewater treatment. Though non-specific binding to XAD is observed, selective removal of nickel and silver (ppm metal removal on top and % metal removal on bottom) is demonstrated by Ybt-XAD and anthra-Ybt-XAD, respectively, including 100% silver removal by anthra-Ybt-XAD.

FIG. 12. Biological characterization of Ybt and Ybt-modified XAD on mammalian cellular viability across three cell lines (RAW264.7 [macrophage], HeLa [epithelial], and NIH3T3 [fibroblast]).

FIG. 13. Example of heterologous biosynthesis of Ybt. A) Introduction of the Irp9 enzyme to enable endogenous production of salicylate within E. coli strain BAP1 in support of heterologous Ybt biosynthesis; B) Qualitative comparison of analog production between two plasmid constructs, one with pBP205 (bottom) and one without pBP205 (top); C) Time course of salicylate produced from BAP1/pCDFDuet-1-irp9 and Ybt produced from BAP1/pBP198/pBP205/pBP200/pCDFDuet-1-irp9; D) Salicylate production with and without the irp9 gene (top). Ybt production comparison between BAP1/pBP198/pBP205+1 mM salicylate and BAP1/pBP198/pBP205/pCDFDuet-1-irp9 (bottom); E) Ybt stability over time as assessed by percent degradation of the compound was analyzed via HPLC upon addition of 64 mg/liter of Ybt-Fe³⁺ to either M9 medium or M9 medium containing strain BAP1 harboring background plasmids (without inserted biosynthetic genes so as to allow the same antibiotic selection; designated “M9 culture”). The samples were then incubated at 22° C. with shaking and IPTG addition. pBP198 contains genes encoding HMWP2, and YbtU and pBP205 contains genes encoding HMWP1 and YbtE.

FIG. 14. A) Comparison between nonmodified XAD particles, particles modified with extract from negative control BAP1/pBP198/pBP200/pCDFDuet-1-irp9, and Ybt-modified XAD across increasing initial copper concentrations. B) Copper removal comparison over time. The initial concentration of copper was 10 ppm. C) Comparison between nonmodified XAD particles and Ybt-modified XAD in a stepwise copper removal process in which fresh beads were added to the same solution to assess copper removal. D) Zinc tested for Ybt sequestration.

FIG. 15. Copper removal of unmodified and Ybt-modified XAD beads across different biological fluids, including RPMI 1640 A), 10% sheep blood in PBS B), and RPMI 1640 plus 50% FBS C). Asterisks indicate statistical significance (95% confidence) in comparison to corresponding negative controls.

FIG. 16. Biological characterization of Ybt and Ybt-modified XAD. Assays included effects on mammalian cellular viability A), RBC hemolysis B), and macrophage immunogenicity C). Controls included methanol and unmodified XAD.

FIG. 17. Precursor-directed biosynthesis summary featuring the substrates fed and the chemical structure and MW expected for final compounds.

FIG. 18. Compound analysis via A) LC-MS and B) LC-MS/MS.

FIG. 19. Example of FadL influence on precursor incorporation into the heterologous biosynthetic system. A fadL knockout version of strain BAP1 was transformed with empty plasmid, plasmids encoding FadL mutants (A77E/S100R, S100R, A77E), and plasmid encoding wild-type FadL and compared to BAP1 in the context of Ybt production with exogenous salicylate addition. Plasmids pBP198 and pBP205 were included in both BAP1 and BAP1::ΔfadL to enable Ybt biosynthesis. *Statistical significance (95% confidence) in comparison to the “Empty plasmid” control.

FIG. 20. Example of FadL influence on precursor-directed Ybt a d analog formation. Strain BAP1/pBP198/pBP205 with (pCDFDuet-fadL) and without (pCDFDuet-1) FadL was tested for biosynthesis with exogenous precursor supplementation. *Statistical significance (95% confidence) in the “pCDFDuet-1” control.

FIG. 21. Example of metabolic engineering to eliminate the need for exogenous precursor addition. A) LC trace of the anthranilate analog (analog 2; via pathway-based precursor support using pCDF-trpDE) and Ybt (via pathway-based precursor support using pCDFDuet-1-irp9. B) MS identification of the anthranilate analog.

FIG. 22. Gold binding comparison. A) LC traces for the anthranilate analog (analog 2) and Ybt in the presence of Au(III). B) The observed peak MS (i) and MS/MS (ii) patterns.

FIG. 23. Maps for examples of plasmids.

FIG. 24. Yersiniabactin 13C-isomer internal standard production characterization. A) LC analysis at 385 nm. B) Resulting MS evaluation. The 556.3 m/z value represents complete ¹²C to ¹³C substitution (21 carbons total in Ybt).

FIG. 25. Example of heterologous biosynthesis and metabolic engineering of yersiniabactin. A) Biosynthesis through the activity of four enzymes (YbtE, HMWP2, HMWP1, YbtU) and the intracellular metabolites salicylate, cysteine, malonyl-CoA, S-adenosylmethionine, and NADPH. Biosynthesis has been reconstituted through E. coli strain BAP1, and metabolic engineering steps have been indicated through the activity of Irp9, MetK, and the ACC enzymes. B) Results of metabolic engineering and exogenous cysteine addition using strain BAP1/pBP198/pBP205 with (1) pCDF-irp9, (2) pMKA-10, (3) pMKA-11, (4) pMKA-11 and pMKA-20, and (5) pMKA-11 and pMKA-20 and 2 mM cysteine addition. Plasmid pBP198 contains genes encoding for HMWP2 and YbtU; pBP205 contains genes encoding for HMWP1 and YbtE.

FIG. 26. Packed bed removal comparison for XAD, Ybt-XAD, and Chelex 100 from solutions of zinc and copper. A) Comparison at pH 6.5 and B) summary of copper removal selectivity.

FIG. 27. Packed-bed column continuous operation and pH regeneration at time points 6 and 12 hrs.

FIG. 28. Map of constructed plasmids: pMKA-10 with irp9 and ybtT under the same T7 promoter using the pCDFDuet-1 vector as the backbone plasmid; pMKA-11 is similar to pMKA-10 with an extra metK gene under a separate T7 promoter; pMKA-20 contains accABCD in a pseudo-operon configuration in which each gene is under a separate T7 promoter on the pACYCDuet-1 vector. YbtT is included to aid biosynthesis through a proposed biosynthetic editing mechanism.

FIG. 29. Example of Ybt-XAD adsorption analysis and packed-bed configuration. A) Ybt-XAD copper adsorption is governed by a Langmuir model and linearization B) provides Q0 and K values. C) Adsorption kinetics of Ybt-XAD for three different initial concentrations of copper. qe is amount of copper (mg) adsorbed per unit weight of resin (g) at equilibrium (60 minutes); Ce is equilibrium concentration of copper remaining in solution when amount adsorbed equals qe. D) The column used for copper removal.

FIG. 30. Packed-bed removal experiments at pH 4.5 and 9.

FIG. 31. Ybt degradation over time for pH 0 (1000 mM HCl), 1 (100 mM HCl), and 2 (10 mM HCl).

FIG. 32. Reduced concentration of precious metals in waste water effluent can add up to significant annual savings.

FIG. 33. Schematic of a constructed model. Waste water stream with 3.2 ppm copper concentration was entering either a commercial resin pack-bed column or a pack-bed column filled with our developed resin.

FIG. 34. A. Schematic of a constructed model. Waste water stream with 3.2 ppm copper concentration was first entering an activated carbon pack-bed column. The stream then enters either a commercial resin pack-bed column or a pack-bed column filled with our developed resin. B. Copper content before and after treatment in each step was measured using a Zincon assay. Activated carbon can remove copper up to 75%. Using additional scavenger step can remove 95 to 100% of copper.

FIG. 35. Schematic of an example of a large scale operational unit for precious metal removal from wastewater streams. Purpose of backwash is to concentrate metal content by at least 1000 times.

FIG. 36. Constructed model. Operating units and wastewater flow are marked.

### DESCRIPTION OF THE DISCLOSURE

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The present disclosure provides Ybt compounds (which include Ybt analogs (and derivatives thereof)) and compositions comprising Ybt and/or Ybt compounds. Methods of making Ybt and Ybt compounds and methods of using Ybt and/or Ybt compounds are also disclosed herein.

The present disclosure includes all polynucleotide and amino acid sequences described herein, and every polynucleotide sequence referred to herein includes its complementary DNA sequence, and also includes the RNA equivalents thereof to the extent an RNA sequence is not given. Every DNA and RNA sequence encoding polypeptides disclosed herein is encompassed by this disclosure. All polynucleotide and amino acid sequences encompassed by this disclosure include polynucleotide and amino acid sequences that are at least 80%-99% identical to such sequences. The identify may be across the entire length of the sequences, or across any contiguous segment of them.

Production of the mixed nonribosomal peptide-polyketide natural product yersiniabactin (Ybt) was established using E. coli. In the present disclosure, precursor-directed biosynthesis was used to generate new analogs of Ybt. A combination of biosynthetic and cellular engineering was used to influence the production metrics of the resulting analogs, as described further herein.

Ybt and its analogs can be used to recover metal from, for example, electroplating processes. Accordingly, there is a significant application given the volume of electronic waste (i.e., discarded electronic devices) and the electroplated metal content of this waste destined for environmental contamination. The degree of metal scavenging of Ybt and its inventive analogs is greater than existing scavengers. Ybt and its analogs can also be used as corrosion inhibitors, rust removers, biofilm formation inhibitors and metal removers/recoverers, as well as in biosensors, bioremediation, and antimicrobial approaches. The compounds of the subject disclosure which selectively bind copper may be used in treating, for example, Wilson's disease, in which an excess of this metal accumulates in tissue. Similarly, Ybt and its analogs that bind iron can be used to treat thalassemia or other iron overload diseases that bind iron. Ybt and its analogs can also be used to treat any other metal-overload conditions. Ybt and its analogs can also act as Trojan horses by linking the Ybt or analogs to antibodies or other targeting agents, or the Ybt or its analogs may themselves target a particular component, such as a cellular receptor to which the Ybt or analog binds.

The list of representative siderophore compounds (e.g., Ybt and Ybt analogs) provided herein has a common biosynthetic feature in that all derive from the action of nonribosomal peptide synthetases with occasional contributions from polyketide synthase enzymes. The modular, diverse, and flexible nature of these enzymes offer numerous possibilities for directed manipulation of the biosynthetic pathways for the purpose of new enzymatic insight and final compound molecular variation. In addition, the growing number of successful cases of heterologous production of such compounds provides parallel opportunities to study biosynthesis within a surrogate host.

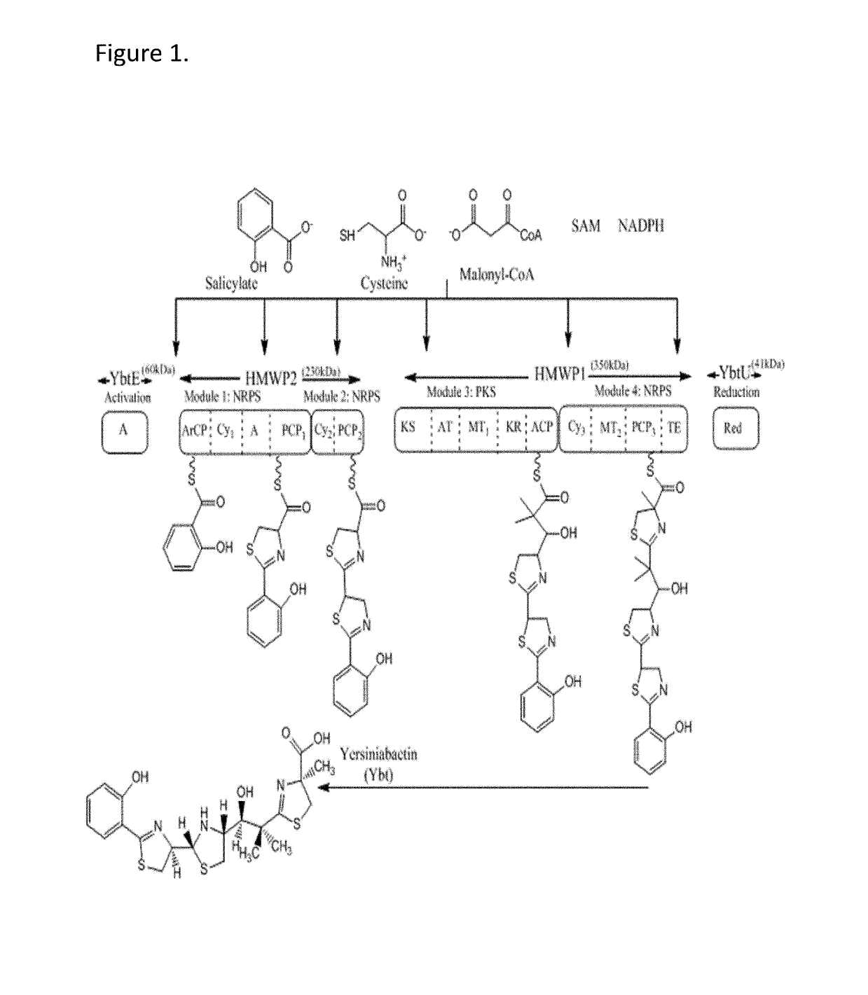

In the present disclosure, Ybt and its analogs are synthesized in a heterologous host. FIG. 1 provides an overview of the inventive biosynthetic process.

As used herein, the term “alkyl,” unless otherwise stated, refers to branched or unbranched hydrocarbons. Examples of such alkyl groups include methyl groups, ethyl groups, propyl groups, butyl groups, isopropyl groups, tert-butyl groups, and the like. For example, the alkyl group is a C₁ to C₆ alkyl group, including all integer numbers of carbons and ranges of numbers of carbons therebetween. Alkyl groups can be substituted with various other functional groups. For example, the alkyl groups can be substituted with groups such as, for example, amines (acyclic and cyclic), alcohol groups, ether groups, thiol groups, and halogen atoms.

Throughout this application, “Ybt or its analogs” refers to Ybt, an analog thereof, a combination of Ybt analogs or a combination of Ybt and Ybt analog(s). Throughout this application, “Ybt analogs” refers to Ybt analogs, derivatives thereof, a combination of Ybt analogs and derivatives thereof.

In an aspect, the present disclosure provides Ybt analogs and compositions comprising Ybt analogs. Ybt analogs can be made using methods described herein. Accordingly, in an example Ybt analogs are made by a method of the present disclosure. In an example, the compound of the present disclosure is not Ybt.

The Ybt analogs can have, for example, the following structure:

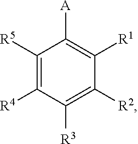

where R¹ is OH, or NH₂, R², R³, R⁴, and R⁵ are each independently H, OH, NH₂, SH, I, Br, Cl, F, or C₁-C₆ alkyl or one or more adjacent combinations of R², R³, R⁴, and/or R⁵ that together form one or more fused rings (e.g., naphthalene moieties, anthracene moieties, phenanthrene moieties, and substituted analogs thereof), and A is selected from the group consisting of:

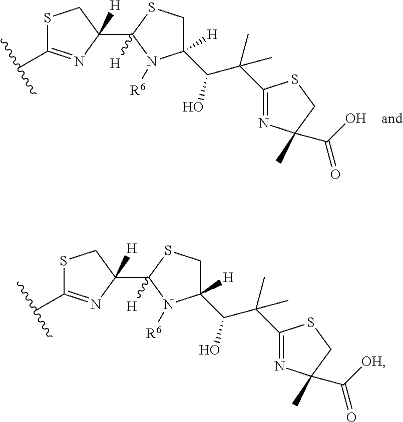

where R⁶ is H or CH₃. In an example, the compound has one of the preceding structures with the proviso that when R², R³, R⁴, and R⁵ are H, R¹ is not OH. In an example, R⁶ is H. In another example, the Ybt analog is not Ybt.

In various examples, the compounds of the present disclosure are selected from the group consisting of:

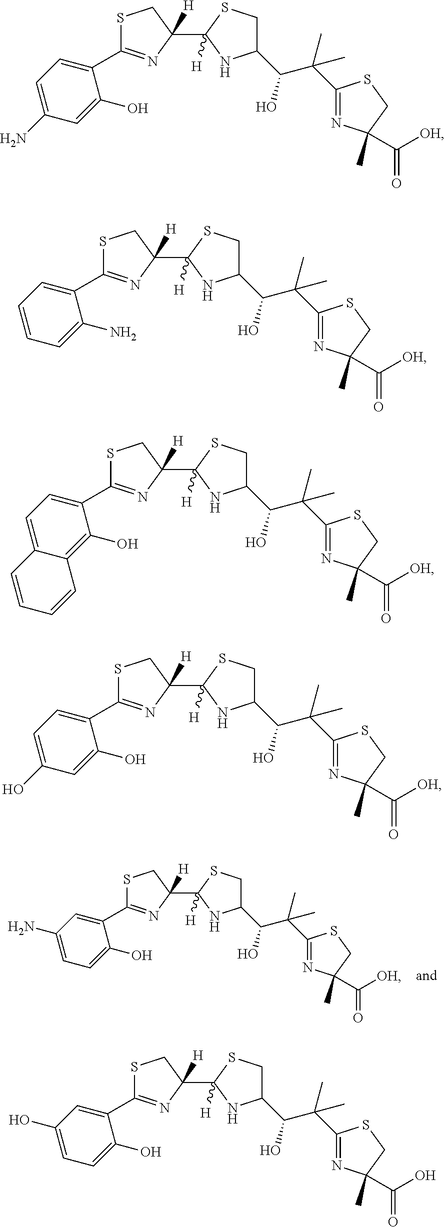

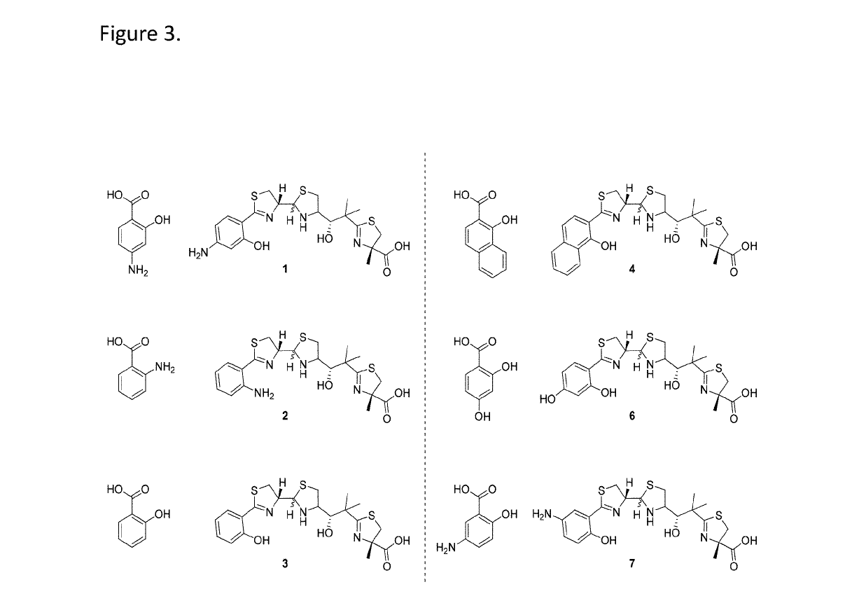

Without intending to be bound by any theory, it is believed that compounds of the subject invention that have a fused ring structure are effective in corrosion inhibition due to the increased surface area. Accordingly, analog 4 (FIG. 3) is particularly effective in corrosion inhibition.

When two NH₂ groups are present, a polymer may be formed. Accordingly, in an example, a polymer comprises (or consists of) repeat units (Ybt and/or Ybt analog moieties) derived from a compound of the present disclosure.

Ybt and/or Ybt analogs of the present disclosure can be bound (covalently or non-covalently (e.g., ionically bonded or bonded via hydrophobic interaction between the Ybt or Ybt analogs and the solid support)) to a solid support. Accordingly, in an example, a solid support comprises one or more Ybt and/or Ybt analogs, where the one or more Ybt and/or Ybt analogs are bound to one or more exterior surface of the solid support.

The support can comprise organic and/or inorganic materials. In various examples, Ybt and/or Ybt analogs are attached to a polymeric bead, polymeric resin, non-polymeric resin, activated carbon, or inorganic material. In various examples, the polymeric bead comprises a non-ionic crosslinked polymer or a hydrophobic resin. In various examples, the organic support materials are hydrophobic polyaromatic resins (e.g., styrene-divinyl benzene copolymer resins) such as Amberlite® XAD® resins (e.g., Amberlite® XAD16 resins), which are commercially available, for example, from Sigma-Aldrich.

Ybt and/or Ybt analogs of the present disclosure can be conjugated to a targeting ligand or a ligand that improves cell permeability. Accordingly, in an example, Ybt or a Ybt analog of the present disclosure is conjugated to a targeting ligand or a ligand that improves cell permeability. Acceptable ligands of the present disclosure include, but are not limited to, folate, biotin, amino acids, peptides, and proteins (e.g., antibodies). Ligands can be conjugated (e.g., covalently bonded) connected to Ybt analogs via linking moieties such as disulfide bonds or groups that can be metabolized in vivo to release the active compound. Alternatively, ligands can be directly bonded to Ybt or Ybt analogs of the present disclosure, for example, via a direct covalent bond between the compound and the ligand (e.g., a bond between a primary amine of the ligand and the carboxylic acid of the compound resulting in an amide linkage). Examples of conjugation methodology (e.g., direct linking methodology and linking methodology) are known in the art.

The Ybt analogs of the present disclosure and Ybt can coordinate a metal (e.g., a metal ion) to provide a metal-coordinated Ybt analog or metal-coordinated Ybt. Accordingly, in an example, a Ybt analog comprises a metal coordinated to the Ybt analog and/or a Ybt comprises a metal coordinated to the Ybt. The metal coordination can be reversible. For example, metal coordination to a Ybt analog or Ybt can be reversed (to provide a metal-free Ybt analog or metal-free Ybt) by changing the environment in which the metal-coordinated Ybt analog or metal-coordinated Ybt resides (e.g., changing the pH of the environment).

The present disclosure includes all possible stereoisomers and geometric isomers of Ybt or Ybt analogs of the present disclosure. The present disclosure includes both racemic compounds and optically active isomers. When a compound of the present disclosure is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetry, 8(6), pages 883-888 (1997). Resolution of the final product, an intermediate, or a starting material can be achieved by any suitable method known in the art. Additionally, in situations where tautomers of a compound of the present disclosure are possible, the present disclosure is intended to include all tautomeric forms of the compounds.

Ybt or Ybt analogs the present disclosure can exist as salts. Accordingly, in an example, Ybt or Ybt analogs is a salt. The salts can be pharmaceutically acceptable salts. As used herein, the term “pharmaceutically acceptable salts” refers to salts or zwitterionic forms of a compound of the present disclosure. Salts of compounds of the present disclosure can be prepared during the final isolation and purification of the compounds or separately by reacting the compound with an acid having a suitable cation. The pharmaceutically acceptable salts of a compound of the present disclosure are acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of this disclosure include, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulphonate, and p-toluenesulfonate salts. In addition, available amino groups present in the compounds of this disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference to compounds of the present disclosure appearing herein is intended to include a compound of the present disclosure as well as pharmaceutically acceptable salts, and hydrates.

A composition can comprise Ybt and/or Ybt analogs the present disclosure. A pharmaceutical composition can comprise one or more of Ybt and/or Ybt analogs this disclosure. Using techniques and carriers known to those of skill in the art (e.g., Remington: The Science and Practice of Pharmacy (2005) 21st Edition, Philadelphia, Pa. Lippincott Williams & Wilkins), the Ybt and its analogs can be formulated as a rinse, gel, drops, spray, cream, toothpaste, lozenge, gum, candy, chocolate, or orally-disintegrating tablet (ODT). Ybt and its analogs could also be made into a tablet, caplet, hard or soft capsule, solution, suspension, emulsion, powder, suppository. Pharmaceutically acceptable carriers include, for example, solvents, emulsifiers, suspending agents, sweeteners and flavorings. The pharmaceutical composition may also be formulated for controlled or modified release using techniques and carriers known to those of skill in the art (e.g., Remington: The Science and Practice of Pharmacy (2005) 21st Edition, Philadelphia, Pa. Lippincott Williams & Wilkins).

Compositions of the present disclosure can comprise more than one pharmaceutical agent. For example, a first composition comprising a compound of the present disclosure and a first pharmaceutical agent can be separately prepared from a composition which comprises the same compound of this disclosure and a second pharmaceutical agent, and such preparations can be mixed to provide a two-pronged (or more) approach to achieving the desired prophylaxis or therapy in an individual. Further, compositions of this disclosure can be prepared using mixed preparations of any of the compounds disclosed herein. In certain aspects an effective amount of a pharmaceutical composition of this disclosure is administered to an individual in need thereof.

In an aspect, the present disclosure provides modified microorganisms including, but not necessarily limited to, bacteria, which are modified such that they expresses heterologous genes suitable to produce the Ybt analogs described herein and methods of making such modified microorganisms. This disclosure is illustrated using Escherichia coli but other microbes are also suitable and include but are not necessarily limited to instead Bacillus subtilis, Streptomyces coelicolor, Streptomyces lividans, Streptomyces venezuelae, Saccharomyces cerevisiae, Pichia pastoris, Aspergillus niger, Aspergillus nidulans, myxobacteria, and Pseudomonas spp. The modified microbes can accordingly also comprise the precursors used to produce the analogs and/or the analogs themselves. In certain approaches the modified microbes are altered to express heterologous proteins. The heterologous proteins expressed by the modified microbes for the purpose of producing Ybt or analogs can comprise or consist of proteins endogenously produced by Yersinia pestis. In examples, modified bacteria of this disclosure express a combination of Y. pestis proteins or modified versions thereof that comprise or consist of Y. pestis HMWP1 protein (encoded by the Y. pestis irp1 gene), HMWP2 protein (encoded by the Y. pestis irp2 gene), salicyl-AMP ligase (encoded by the Y. pestis ybtE gene), thiazolinyl-S-HMWP1 reductase (encoded by the Y. pestis ybtU gene), putative phosphopantetheinyl transferase (encoded by the Y. pestis ybtD gene), for which the Bacillus subtilis sfp gene is a suitable alternative. The sequence of the Bacillus subtilis sfp gene and its encoded protein is well known in the art. In one embodiment the sfp gene may be integrated into the chromosome of a microbe, such as E. coli. The Sfp protein can be used to activate the HMWP1 and HMWP2 enzymes via phosphopantetheinylation. This disclosure includes the proviso that modified microorganisms of this disclosure in certain examples do express a salicylate synthase, including but not necessarily limited to the salicylate synthase encoded by the Y. pestis Irp9 (ybtS) gene. Thus the microorganisms can lack the irp9 gene and/or any protein coding region of it. This disclosure also includes the proviso that in certain examples the modified microorganism does not express a yersiniabactin biosynthetic protein produced by, for example, the Y. pestis YbtT gene. In certain approaches modified microorganisms of this disclosure may be modified to produce an excess of precursors which will lead to increased production of either Ybt or analogs. Overexpression of any one or any combination of the genetic modifications described herein can be achieved using standard approaches, including but not necessarily limited to using strong promoters, or increasing copy number of the genes. These include but are not limited to overexpression of malonyl-CoA support genes (e.g., accABCD), enhanced intracellular levels of NADH and ATP, cysteine and S-adenosylmethionine overproduction. It is also possible to improve precursor levels by adding exogenous substrates such as cysteine to the cell culture production medium.

In certain examples the modified bacteria express via an episomal element, or overexpress, the E. coli long-chain fatty acid transport protein (fadL) because the resulting protein product is expected to aid the transport of those precursors utilized during precursor-directed efforts to generate Ybt analogs.

The following amino acid sequences are pertinent to the present disclosure:

HMWP1 amine sequence (encoded by Y. pestis irp1
gene)
(SEQ ID NO: 1)
MDNLRFSSAPTADSIDASIAQHYPDCEPVAVIGYACHFPESPDGETFWQN
LLEGRECSRRFTREELLAVGLDAAIIDDPHYVNIGTVLDNADCFDATLFG
YSRQEAESMDPQQRLFLQAVWHALEHAGYAPGAVPHKTGVFASSRMSTYP
GREALNVTEVAQVKGLQSLMGNDKDYIATRAAYKLNLHGPALSVQTACSS
SLVAVHLACESLRAGESDMAVAGGVALSFPQQAGYRYQPGMIFSPDGHCR
PFDASAEGTWAGNGLGCVVLRRLRDALLSGDPIISVILSSAVNNDGNRKV
GYTAPSVAGQQAVIEEALMLAAIDDRQVGYIETHGTGTPLGDAIEIEALR
NVYAPRPQDQRCALGSVKSNMGHLDTAAGIAGLLKTVLAVSRGQIPPLLN
FHTPNPALKLEESPFTIPVSAQAWQDEMRYAGVSSFGIGGTNCHMIVASL
PDALNARLPNTDSGRKSTALLLSAASDSALRRLATDYAGALRENADASSL
AFTALHARRLDLPFRLAAPLNRETAEALSAWAGEKSGALVYSGHGASGKQ
VWLFTGQGSHWRTMGQTMYQHSTAFADTLDRCFSACSEMLTPSLREAMFN
PDSAQLDNMAWAQPAIVAFEIAMAAHWRAEGLKPDFATGHSVGEFAAAVV
CGHYTIEQVMPLVCRRGALMQQCASGAMVAVFADEDTLMPLARQFELDLA
ANNGTQHTVFSGPEARLAVFCATLSQHDINYRRLSVTGAAHSALLEPILD
RFQDACAGLHAEPGQIPIISTLTADVIDESTLNQADYWRRHMRQPVRFIQ
SIQVAHQLGARVFLEMGPDAQLVACGQREYRDNAYWIASARRNKEASDVL
NQALLQLYAAGVALPWADLLAGDGQRIAAPCYPFDTERYWKERVSPACEP
ADAALSAGLEVASRAATALDLPRLEALKQCATRLHAIYVDQLVQRCTGDA
IENGVDAMTIMRRGRLLPRYQQLLQRLLNNCVVDGDYRCTDGRYVRARPI
EHQQRESLLTELAGYCEGFQAIPDTIARAGDRLYEMMSGAEEPVAIIFPQ
SASDGVEVLYQEFSFGRYFNQIAAGVLRGIVQTRQPRQPLRILEVGGGTG
GTTAWLLPELNGVPALEYHFTDISALFTRRAQQKFADYDFVKYSELDLEK
EAQSQGFQAQSYDLIVAANVIHATRHIGRTLDNLRPLLKPGGRLLMREIT
QPMRLFDFVFGPLVLPLQDLDAREGELFLTTAQWQQQCRHAGFSKVAWLP
QDGSPTAGMSEHIILATLPGQAVSAVTFTAPSEPVLGQALTDNGDYLADW
SDCAGQPERFNARWQEAWRLLSQRHGDALPVEPPPVAAPEWLGKVRLSWQ
NEAFSRGQMRVEARHPTGEWLPLSPAAPLPAPQTHYQWRWTPLNVASIDH
PLTFSFSAGTLARSDELAQYGIIHDPHASSRLMIVEESEDTLALAEKVIA
ALTASAAGLIVVTRRAWRVEENEALSASHHALWALLRVAANEQPERLLAA
IDLAENTPWETLHQGLSAVSLSQRWLAARGDTLWLPSLAPNTGCAAELPA
NVFTGDSRWHLVTGAFGGLGRLAVNWLREKGARRIALLAPRVDESWLRDV
EGGQTRVCRCDVGDAGQLATVLDDLAANGGIAGAIHAAGVLADAPLQELD
DHQLAAVFAVKAQAASQLLQTLRNHDGRYLILYSSAAATLGAPGQSAHAL
ACGYLDGLAQQFSTLDAPKTLSVAWGAWGESGRAATPELATLASRGMGAL
SDAEGCWHLEQAVMRGAPWRLAMRVFTDKMPPLQQALFNISATEKAATVI
PPADDNAFNGSLSDETAVMAWLKKRIAVQLRLSDPASLHPNQDLLQLGMD
SLLFLELSSDIQHYLGVRINAERAWQDLSPHGLTQLICSKPEATPAASQP
EVLRHDADERYAPFPLTPIQHAYWLGRTHLIGYGGVACHVLFEWDKRHDE
FDLAILEKAWNQLIARHDMLRMVVDADGQQRILATTPEYHIPRDDLRALS
PEEQRIALEKRRHELSYRVLPADQWPLFELVVSEIDDCHYRLHMNLDLLQ
FDVQSFKVMMDDLAQVWRGETLAPLAITFRDYVMAEQARRQTSAWHDAWD
YWQEKLPQLPLAPELPVVETPPETPHFTTFKSTIGKTEWQAVKQRWQQQG
VTPSAALLTLFAATLERWSRTTTFTLNLTFFNRQPIHPQINQLIGDFTSV
TLVDENFSAPVTLQEQMQQTQQRLWQNMAHSEMNGVEVIRELGRLRGSQR
QPLMPVVFTSMLGMTLEGMTIDQAMSHLFGEPCYVFTQTPQVWLDHQVME
SDGELMFSWYCMDNVLEPGAAEAMENDYCAILQAVIAAPESLKTLASGIA
GHIPRRRWPLNAQADYDLRDIEQATLEYPGIRQARAEITEQGALTLDIVM
ADDPSPSAAMPDEHELTQLALPLPEQAQLDELEATWRWLEARALQGIAAT
LNRHGLFTTPEIAHRFSAIVQALSAQASHQRLLRQWLQCLTEREWLIREG
ESWRCRIPLSEIPEPQEACPQSQWSQALAQYLETCIARHDALFSGQCSPL
ELLFNEQHRVTDALYRDNPASACLNRYTAQTAALCSAERILEVGAGTAAT
TAPVLKATRNTRQSYHFTDVSAQFLNDARARFHDESQVSYALFDINQPLD
FTAHPEAGYDLIVAVNVLHDASHVVQTLRRLKLLLKAGGRLLIVEATERN
SVFQLASVGFIEGLSGYRDFRRRDEKPMLTRSAWQEVLVQAGFANELAWP
AQESSPLRQHLLVARSPGVNRPDKKAVSRYLQQRFGTGLPILQIRQREAL
FTPLHAPSDAPTEPAKPTPVAGGNPALEKQVAELWQSLLSRPVARHHDFF
ELGGDSLMATRMVAQLNRRGIARANLQDLFSHSTLSDFCAHLQAATSGED
NPIPLCQGDGEETLFVFHASDGDISAWLPLASALNRRVFGLQAKSPQRFA
TLDQMIDEYVGCIRRQQPHGPYVLAGWSYGAFLAAGAAQRLYAKGEQVRM
VLIDPVCRQDFCCENRAALLRLLAEGQTPLALPEHFDQQTPDSQLADFIS
LAKTAGMVSQNLTLQAAETWLDNIAHLLRLLTEHTPGESVPVPCLMVYAA
GRPARWTPAETEWQGWINNADDAVIEASHWQIMMEAPHVQACAQHITRWL
CATSTQPENTL
HMWP2 sequence (encoded by Y. pestis irp2 gene)
(SEQ ID NO: 2)
MISGAPSQDSLLPDNRHAADYQQLRERLIQELNLTPQQLHEESNLIQAGL
DSIRLMRWLHWFRKNGYRLTLRELYAAPTLAAWNQLMLSRSPENAEEETP
PDESSWPNMTESTPFPLTPVQHAYLTGRMPGQTLGGVGCHLYQEFEGHCL
TASQLEQAITTLLQRHPMLHIAFRPDGQQVWLPQPYWNGVTVHDLRHNDA
ESRQAYLDALRQRLSHRLLRVEIGETFDFQLTLLPDNRHRLHVNIDLLIM
DASSFTLFFDELNALLAGESLPAIDTRYDFRSYLLHQQKINQPLRDDARA
YWLAKASTLPPAPVLPLACEPATLREVRNTRRRMIVPATRWHAFSNRAGE
YGVTPTMALATCFSAVLARWGGLTRLLLNITLFDRQPLHPAVGAMLADFT
NILLLDTACDGDTVSNLARKNQLTFTEDWEHRHWSGVELLRELKRQQRYP
HGAPVVFTSNLGRSLYSSRAESPLGEPEWGISQTPQVWIDHLAFEHHGEV
WLQWDSNDALFPPALVETLFDAYCQLINQLCDDESAWQKPFADMMPASQR
AIRERVNATGAPIPEGLLHEGIFRIALQQPQALAVTDMRYQWNYHELTDY
ARRCAGRLIECGVQPGDNVAITMSKGAGQLVAVLAVLLAGAVYVPVSLDQ
PAARREKIYADASVRLVLICQHDASAGSDDIPVLAWQQAIEAEPIANPVV
RAPTQPAYIIYTSGSTGTPKGVVISHRGALNTCCDINTRYQVGPHDRVLA
LSALHFDLSVYDIFGVLRAGGALVMVMENQRRDPHAWCELIQRHQVTLWN
SVPALFDMLLTWCEGFADATPENLRAVMLSGDWIGLDLPARYRAFRPQGQ
FIAMGGATEASIWSNACEIHDVPAHWRSIPYGFPLTNQRYRVVDEQGRDC
PDWVPGELWIGGIGVAEGYFNDPLRSEQQFLTLPDERWYRTGDLGCYWPD
GTIEFLGRRDKQVKVGGYRIELGEIESALSQLAGVKQATVLAIGEKEKTL
AAYVVPQGEAFCVTDHRNPALPQAWHTLAGTLPCCAISPEISAEQVADFL
QHRLLKLKPGHTAGADPLPLMNSLAIQPRWQAVVERWLAFLVTQRRLKPA
AEGYQVCAGEEREDEHPHFSGHDLTLSQILRGARNELSLLNDAQWSPESL
AFNHPASAPYIQELATICQQLAQRLQRPVRLLEVGTRTGRAAESLLAQLN
AGQIEYVGLEQSQEMLLSARQRLAPWPGARLSLWNADTLAAHAHSADIIW
LNNALHRLLPEDPGLLATLQQLAVPGALLYVMEFRQLTPSALLSTLLLTN
GQPEALLHNSADWAALFSAAAFNCQHGDEVAGLQRFLVQCPDRQVRRDPR
QLQAALAGRLPGWMVPQRIVFLDALPLTANGKIDYQALKRRHTPEAENPA
EADLPQGDIEKQVAALWQQLLSTGNVTRETDFFQQGGDSLLATRLTGQLH
QAGYEAQLSDLFNHPRLADFAATLRKTDVPVEQPFVHSPEDRYQPFALTD
VQQAYLVGRQPGFALGGVGSHFFVEFEIADLDLTRLETVWNRLIARHDML
RAIVRDGQQQVLEQTPPWVIPAHTLHTPEEALRVREKLAHQVLNPEVWPV
FDLQVGYVDGMPARLWLCLDNLLLDGLSMQILLAELEHGYRYPQQLLPPL
PVTFRDYLQQPSLQSPNPDSLAWWQAQLDDIPPAPALPLRCLPQEVETPR
FARLNGALDSTRWHRLKKRAADAHLTPSAVLLSVWSTVLSAWSAQPEFTL
NLTLFDRRPLHPQINQILGDFTSLMLLSWHPGESWLHSAQSLQQRLSQNL
NHRDVSAIRVMRQLAQRQNVPAVPMPVVETSALGFEQDNFLARRNLLKPV
WGISQTPQVWLDHQIYESEGELRFNWDEVAALFPAGQVERQFEQYCALLN
RMAEDESGWQLPLAALVPPVKHAGQCAERSPRVCPEHSQPHIAADESTVS
LICDAFREVVGESVTPAENFFEAGATSLNLVQLHVLLQRHEFSTLTLLDL
FTHPSPAALADYLAGVATVEKTKRPRPVRRRQRRI
Salicyl-AMP ligase (encoded by Y. pestis ybtE
gene)
(SEQ ID NO: 3)
MNSSFESLIEQYPLPIAEQLRHWAARYASRIAVVDAKGSLTYSALDAQVD
ELAAGLSSLGLRSGEHVIVQLPNDNAFVTLLFALLRLGVIPVLAMPSQRA
LDIDALIELAQPVAYVIHGENHAELARQMAHKHACLRHVLVAGETVSDDF
TPLFSLHGERQAWPQPDVSATALLLLSGGTTGTPKLIPRRHADYSYNFSA
SAELCGISQQSVYLAVLPVAHNFPLACPGILGTLACGGKVVLTDSASCDE
VMPLIAQERVTHVALVPALAQLWVQAREWEDSDLSSLRVIQAGGARLDPT
LAEQVIATFDCTLQQVFGMAEGLLCFTRLDDPHATILHSQGRPLSPLDEI
RIVDQDENDVAPGETGQLLTRGPYTISGYYRAPAHNAQAFTAQGFYRTGD
NVRLDEVGNLHVEGRIKEQINRAGEKIAAAEVESALLRLAEVQDCAVVAA
PDTLLGERICAFIIAQQVPTDYQQLRQQLTRMGLSAWKIPDQIEFLDHWP
LTAVGKIDKKRLTALAVDRYRHSAQ
Thiazolinyl-S-HMWP1 reductase (encoded by Y.
pestis ybtU gene)
(SEQ ID NO: 4)
MCATHYALALRNLNATGEHVMMPSASPKQRVLIVGAKFGEMYLNAFMQPP
EGLELVGLLAQGSARSRELAHAFGIPLYTSPEQITRMPDIACIVVRSTVA
GGTGTQLARHFLTRGVHVIQEHPLHPDDISSLQTLAQEQGCCYWVNTFYP
HTRAGRTWLRDAQQLRRCLAKTPPVVHATTSRQLLYSTLDLLLLALGVDA
AAVECDVVGSFSDFHCLRLFWPEGEACLLLQRYLDPDDPDMHSLIMHRLL
LGWPEGHLSLEASYGPVIWSSSLEVADHQENAHSLYRRPEILRDLPGLTR
SAAPLSWRDCCETVGPEGVSWLLHQLRSHLAGEHPPAACQSVHQIALSRL
WQQILRKTGNAEIRRLTPPHHDRLAGFYNDDDKEAL
Putative phosphopantetheinyl transferase (encoded
by Y. pestis ybtD)
(SEQ ID NO: 5)
MSVLNIPLLPPFITHASLQPLDGHPCVDRLDVREDAGHETPSLFSTLDIP
CPAHLERSVNKRRAEYLVSRYGLQQALASLGIARFVLGNDENRAPIWPAG
IAGSLSHTHQQVCALLTRDDNLLLGIDCEQMMTLDTASEMQSMLINQRER
ERLEQCSLPFNHALTVVESLKESLYKALYPRLKQFMDFSAAEVMECYPDM
QQVSLRLTQTFSAEMVAGRVFTGHAVLQPDRVMTWVVEPQPR
Salicylate synthase (encoded by Y. pestis Irp9
(ybtS) gene):
(SEQ ID NO: 6)
MKISEFLHLALPEEQWLPTISGVLRQFAEEECYVYERPPCWYLGKGCQAR
LHINADGTQATFIDDAGEQKWAVDSIADCARREMAHPQVKGRRVYGQVGE
NFAAHARGIAFNAGEWPLLTLTVPREELIFEKGNVTVYADSADGCRRLCE
WVKEASTTTQNAPLAVDTALNGEAYKQQVARAVAEIRRGEYVKVIVSRAI
PLPSRIDMPATLLYGRQANTPVRSFMFRQEGREALGESPELVMSVTGNKV
VTEPLAGTRDRMGNPEHNKAKEAELLHDSKEVLEHILSVKEAIAELEAVC
LPGSVVVEDLMSVRQRGSVQHLGSGVSGQLAENKDAWDAFTVLFPSITAS
GIPKNAALNAIMQIEKTPRELYSGAILLLDDTRFDAALVLRSVFQDSQRC
WIQAGAGIIAQSTPERELTETREKLASIAPYLMV.
The YbtT amino acid encoded by Y. pestis is:
(SEQ ID NO: 7)
MTQSAMCIPLWPARNGNTAHLVMCPFAGGSSSAFRHWQAEQLTDCALSLV
TWPGRDRLRHLEPLRSITQLAALLANELEASVSPDTPLLLAGHSMGAQVA
FETCRLLEQRGLAPQGLIISGCHAPHLHSERQLSHRDDADFIAELIDIGG
CSPELRENQELMSLFLPLLRADFYATESYHYDSPDVCPPLRTPALLLCGS
HDREASWQQVDAWRQWLSHVTGPVVIDGDHFYPIQQARSFFTQIVRHFPH
AFSAMTALQKQPSTSER
The FadL amino acid sequence is:
(SEQ ID NO: 8)
MVMSQKTLFTKSALAVAVALISTQAWSAGFQLNEFSSSGLGRAYSGEGAI
ADDAGNVSRNPALITMFDRPTESAGAVYIDPDVNISGTSPSGRSLKADNI
APTAWVPNMHEVAPINDQFGWGASITSNYGLATEFNDTYAGGSVGGTTDL
ETMNLNLSGAYRLNNAWSFGLGFNAVYVRAKIERFAGDLGQLVAGQIMQS
PAGQTQQGQALAATANGIDSNTKIAHLNGNQWGFGWNAGILYELDKNNRY
ALTYRSEVKIDFKGNYSSDLNRAFNNYGLPIPTATGGRTQSGYLTLNLPE
MWEVSGYNRVDPQWAIHYSLAYTSWSQFQQLKATSTSGDTLFQKHEGFKD
AYRIALGTTYYYDDNWTFRTGIAFDDSPVPAQNRSISIPDQDRFWLSAGT
TYAFNKDASVDVGVSYMHGQSVKINEGPYQFESEGKAWLFGTNFNYAF

Thus, in various examples the present disclosure pertains to modified bacteria which comprise polynucleotides comprising heterologous protein coding sequences, wherein the heterologous protein coding sequences can encode Y. pestis HMWP1 protein, Y. pestis HMWP2 protein, Y. pestis Salicyl-AMP ligase, and Y. pestis Thiazolinyl-S-HMWP1 reductase, and Y. pestis putative phosphopantetheinyl transferase.

In various examples, the polynucleotide(s) encoding the proteins useful for making the Ybt analogs of this disclosure are comprised within one or more expression vectors. Any suitable expression vector can be used and can be selected by one skilled in the art, given the benefit of the present disclosure.

Polynucleotides encoding the proteins may be maintained as episomal elements, or they may be transiently or stably integrated into a chromosome of any particular modified microorganism of this disclosure. In various examples, the expression vector comprises a plasmid.

In various examples, expression of one or more of the proteins is constitutive, or is conditional, such as use of an inducible promoter to drive expression one or more of the proteins at a particular time. In certain examples, proteins are encoded on a single expression vector, or more than one expression vector, including 2, 3, 4 or 5 expression vectors.

A method of making microorganisms capable of producing compounds of the present disclosure comprises introducing into the microorganisms polynucleotides encoding one or more proteins as described herein using any variety of techniques known to those skilled in the art, such as by transformation of the cell with a vector which comprises more than one gene or by transformation with multiple vectors that each comprise one or more inserted gene. In this regard, vectors which are capable of propagation in microorganisms and which are adapted for expressing more than one inserted gene can be prepared using standard molecular biological techniques or purchased commercially. Thus, one or more nucleic acids which comprise a set of genes sufficient to direct the biosynthesis of one or more of the Ybt analogs of this disclosure can be introduced into cells using well known techniques. For example, transformation of bacteria can be achieved using electroporation or by incubation of transformation competent cells with the vector(s) or by heat shock. Such modified microorganisms can be mixed with precursors as described herein, after which a period time sufficient to produce the Ybt analogs is allowed to pass. Subsequently, the Ybt analogs can be separated from the culture media if desired and purified to any desired degree of purity.

The present disclosure includes the modified microorganisms described herein, cell cultures and culture media comprising the modified microorganisms, the cell culture media comprising the analogs of this disclosure, including but not limited to culture media that has been separated from the microorganisms. The present disclosure also includes culturing the modified microorganisms in or on any surface or location wherein sequestration of metal is desirable. The present disclosure includes containers comprising the microorganisms, non-limiting examples of which include vials, petri dishes, tubes such as Eppendorf tubes, test tubes, beakers, flasks, vats, and other containers and vessels that will be apparent to those skilled in the art. The present disclosure includes modified microorganisms that have been preserved, such as being in a mixture with a preservative such as glycerol, wherein the microorganisms may be stored at a reduced temperature, including but not necessarily limited to temperatures below freezing. The microorganisms can comprise within their cells or in their culture medium one or more Ybt analogs described herein. The present disclosure includes kits comprising the microorganisms and precursors that can be used to make the Ybt analogs.

In an aspect, the present disclosure provides methods of making Ybt and/or Ybt compounds (e.g., Ybt analogs and derivatives thereof) of the present disclosure. The methods are biosynthetic methods based on the use of modified microorganisms.

A method of making one or more compounds of the present disclosure can comprise: contacting one or more modified microorganisms (e.g., one or more modified Escherichia coli cells) such that they produce (e.g., secrete) a compound of the present disclosure with a precursor compound having the following structure:

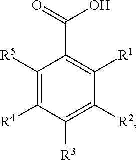

where R¹ is OH, or NH₂, R², R³, R⁴, and R⁵ are each independently H, OH, NH₂, SH, I, Br, Cl, F, C₁-C₆ alkyl, or one or more adjacent combinations of R², R³, R⁴, and/or R⁵ that together form one or more fused rings (e.g., naphthalene moieties, anthracene moieties, phenanthrene moieties, and substituted analogs thereof), for a period of time sufficient to produce the one or more compounds. The microorganisms can be incubated prior to contact with the precursor compound. The precursor compound can be endogenous or exogenous. A method of making one or more compounds of the present disclosure can further comprise isolating the one or more compounds (e.g., physically separating the one or more compounds from the sample).

The contacting is carried out for a predetermined time or until a desired amount of Ybt analog is produced. In various examples, the contacting is carried out for 5 hours to 7 days, including all 0.1 hour values and ranges therebetween.

The contacting can be carried out at various temperatures. Suitable temperatures include those in which the microorganisms are viable. In various examples, the contacting is carried out a temperature of 10° C. to 42° C., including all integer ° C. values and ranges therebetween.

The contacting can be carried out in a reaction mixture. For example, the reaction mixture comprises one or more modified microorganisms (e.g., modified Escherichia coli cells), a precursor compound, and minimal, defined, or complex growth medium. Suitable minimal, defined, or complex growth media are known in the art. In an example, a reaction mixture further comprises a support material.

Supported Ybt and/or Ybt analogs can be formed in situ. For example, in a method of making a Ybt and/or Ybt analog the modified microorganism and precursor compound are contacted in the presence of a support material (e.g., XAD16) and supported Ybt and/or Ybt analogs are formed in situ. Other supports include, but are not limited to, polymeric bead, polymeric resin, non-polymeric resin, activated carbon, and inorganic material.

Various reactors can be used to make Ybt and/or Ybt analogs. Suitable reactors are known in the art. For example, a bioreactor can be used to synthesize Ybt and/or Ybt analogs. The bioreactor can be, for example, a heated stirred tank bioreactor. For example, a 5 L bioreactor can be used, but the volume can vary depending on factors such as the desired production amounts. The height of the impeller above the bottom of the bioreactor can be configured to improve the reaction. The heaters may be resistive heaters or other heater designs known to those skilled in the art.

In an aspect, the present disclosure provide methods of using Ybt and/or Ybt compounds (e.g., Ybt analogs and derivatives thereof), or compositions comprising Ybt and/or Ybt compounds of the present disclosure. In various examples, the methods include metal sequestration by Ybt and/or Ybt compounds (e.g., Ybt analogs) of the present disclosure, or compositions comprising Ybt and/or Ybt analogs of the present disclosure.

For example, Ybt and/or Ybt compounds or compositions comprising Ybt and/or Ybt compounds of the present disclosure can be used in methods for removing rust from a ferrous metal-containing surface. As used herein, “rust” refers to a coating or film formed on a metal by oxidation or corrosion. In some cases, the rust that is removed in the methods of the present invention is “red rust” which, as used herein, refers to a coating or film formed on iron or steel by oxidation, as during exposure to air and/or moisture, that comprises iron (II) oxide (FeO, wustite), alpha phase iron (III) oxide (a-Fe₂O₃, hematite), beta phase iron (III) oxide (P—Fe₂O₃), gamma phase iron (III) oxide (Y—Fe₂O₃, maghemite), epsilon phase iron (III) oxide (s-Fe₂O₃), iron (II) hydroxide (Fe(OH)₂), iron (III) hydroxide (Fe(OH)₃, bernalite), and/or hydrated forms and combinations of any of the foregoing. In some examples, the iron oxide that is removed in the methods of the present invention is of a type that is often referred to as “mill scale” which, as used herein, refers to a coating or film formed on iron or steel by oxidation, as during exposure to air, moisture, and/or heat, that comprises iron(II,III) oxide (Fe₃O₄, magnetite), alpha phase iron (III) oxide (a-Fe₂O₃, hematite), iron(II) hydroxide Fe(OH)₂, (III) hydroxide (Fe(OH)₃, bernalite), and/or hydrated forms and combinations of any of the foregoing.

A method of removing one or more metals from a metal-containing sample can comprise: a) contacting the metal-containing sample with one or more compounds of the present disclosure (Ybt and/or Ybt analogs, which can be supported or unsupported) where a complex is formed between the one or more metals of the metal-containing sample and compound (e.g., between at least a portion of the metal(s)) or for a period of time sufficient to form a complex between the one or more metals of the metal-containing sample (e.g., between at least a portion of the metal(s)); b) optionally, isolating the complex of a) from the sample.

The Ybt and/or Ybt analogs and/or the metal-complexed Ybt and/or Ybt analogs can be removed from (e.g., isolated from) the sample.

The metal complexation can be reversible. Accordingly, in an example, a method of removing one or more metals from a metal-containing sample further comprises optionally removing the metal-complexed Ybt and/or Ybt analogs and regenerating (i.e., forming metal-free Ybt and/or Ybt analogs). The regenerating can be carried out by adjusting the pH of the environment of the metal-complexed Ybt and/or Ybt analogs such that metal-free Ybt and/or Ybt analogs and free metal(s) are formed. The metal-free Ybt and/or Ybt analogs can be isolated from the metal(s) and the metal-free Ybt and/or Ybt analogs reused.

Various metal-containing samples can be used. A metal-containing sample can comprise one or more metals. A metal containing sample can be a solution/suspension comprising one or more metals or a solid sample comprising one or more metals. In various examples the metal-containing sample (prior to contact with one or more compounds of the present disclosure) comprises one or more metals at a concentration 5 ppm or less, 1 ppm or less, 0.5 ppm or less, 0.4 or less, 0.3 or less, 0.2 or less, 0.1 or less, 0.05 or less, or 0.01 or less.

Examples of metal-containing samples include various liquid samples and solid samples. Examples of metal-containing samples include aqueous samples such as water, tap water, pool water, wastewater, ocean water, freshwater and brine), rust-containing surfaces (e.g., jewelry, flatware, aquatic vessels, buildings), various industrial samples (e.g., mine tailings/waste, semiconductor (e.g., semiconductor process) effluent, samples from fish farms (e.g., samples from fish farm growth environments or other aqueous media used for farm-raised fish production), jewelry and plating (e.g., jewelry and/or plating process) effluents, solar panel (e.g., solar panel production) effluents, wastewater treatment sludge or precipitants), soil, electronic waste, and coal that comprise one or more metals.

The metal-containing sample can be contacted with Ybt and/or Ybt analogs under static (batch) or dynamic conditions (e.g., flow conditions such as continuous flow conditions). For example, the metal-containing sample is contacted with Ybt and/or Ybt analogs (e.g., a solution comprising Ybt and/or Ybt analogs) under static (batch). The solution can be an aqueous solution (e.g., an aqueous solution having, for example, a pH of 3 to 10). In another example, the metal-containing sample is contacted with Ybt and/or Ybt analogs (e.g., a solution comprising Ybt and/or Ybt analogs) under dynamic conditions (e.g., flowing the sample or solution that has contacted the sample through supported Ybt and/or Ybt analogs, which can be carried out multiple times (e.g., a recirculating the sample or solution that has contacted the sample)). The solution can be an aqueous solution (e.g., an aqueous solution having, for example, a pH of 3 to 10).

The metal-containing sample can be contacted with Ybt and/or Ybt analogs under various conditions. Variations in the conditions are considered within the scope of the present disclosure. For instance, pH, temperature and other parameters are varied to optimize metal binding and/or release. For example, the metal-containing sample is contacted with Ybt and/or Ybt analogs in an aqueous solution at a pH of 3 to 10, including all 0.1 pH values and ranges therebetween.

The metal-containing sample can be contacted with supported or non-supported Ybt and/or Ybt analogs. Examples of supported Ybt and/or Ybt analogs are described herein. The supported or non-supported Ybt and/or Ybt analogs can be present in a housing. For example, the housing is a cartridge, column, or other standard housing used in a unit operation designated for (e.g., configured for) separation processing.

Various metals can be removed from the metal-containing sample. Examples of a metal (e.g., a metal in the metal-containing sample) include, but are not limited to, iron, copper, gold, silver, palladium, platinum, lithium, aluminum, bismuth, gallium, germanium, indium, rhodium, selenium, silicon, nickel, tellurium, zinc, manganese, arsenic, lead, mercury, cerium, scandium, yttrium, lanthanum, praseodymium, neodymium, promethium, samarium, europium, gadolinium, terbium, dysprosium, holmium, erbium, thulium, ytterbium, lutetium, and combinations thereof. The metal can be in ionic (e.g., cationic) form.

In an example, Ybt (e.g., supported Ybt) is used to remove copper from a metal-containing sample. In a metal-containing sample that comprises copper and zinc, Ybt selectively binds to copper.

The metal-containing sample can be contacted with Ybt and/or Ybt analogs for a predetermined time or until a desired amount of metal is removed from the sample. For example, the metal-containing sample are contacted with Ybt and/or Ybt analogs under static conditions or dynamic conditions for 0.1 minute to 3 hours or until a desired amount of metal is removed from the sample, including all 0.1 minute values and ranges therebetween, at a temperature of 18° C. to 35° C., including all 0.1° C. values and ranges therebetween. The amount of metal removed can be determined by analysis by a method disclosed herein of the sample or an aliquot of the sample or a solution that contacted the sample.

Desirable amounts of metal(s) can be removed from the metal-containing sample. For example, at least 90% of the metal(s) are removed from the sample. In various example, at least 95, 96, 97, 98, 99, 99.5, 99.9, 99.9, 99.99, 99.999, or 99.9999% of the metal(s) are removed from the sample. For example, the metal(s) are quantitatively removed from the sample (i.e., no detectible amounts of metal(s) are present in the sample). The amount of metal removed and/or remaining in the sample can be determined by methods known in the art and methods described herein. For example, the amount of metal removed and/or remaining in the sample are determined by gravimetric methods, chromatographic methods (e.g., liquid chromatorgraphy (LC)) assay methods, and/or spectroscopic methods such as atomic absorption, colorimetric methods, spectrometry methods such as mass spectrometry (MS) methods (e.g., ICP-MS, LC-MS, and LC-MS/MS).

The steps of the methods described herein and in the various examples disclosed herein are sufficient to produce or use the Ybt and Ybt analogs of the present disclosure. Thus, in an various examples, a method consists essentially of a combination of the steps of the methods disclosed herein. In another example, the method consists of such steps.

The following examples are presented to illustrate the present disclosure. They are not intended to be limiting in any manner.

### Example 1

This example describes the biosynthesis, characterization, and use of Ybt and Ybt analogs.

Heterologous biosynthesis was established in E. coli to provide a safe and technically-advanced production platform relative to the native host. In particular, the background E. coli strain was engineered to express a 4′-phosphopantetheine transferase (termed Sfp) which allows for the posttranslational modification of polyketide synthase and nonribosomal peptide synthetase biosynthetic enzymes. The Yersiniabactin biosynthesis uses a modular sequence of reactions that combines the activity of both enzyme types to produce a mixed nonribosomal peptide-polyketide product. The majority of native substrates required for Ybt biosynthesis (malonyl-CoA, NADPH, S-adenosylmethionine, cysteine) are provided by E. coli; whereas, the starting salicylate substrate must either be added exogenously or provided through metabolic engineering of the shikimate and chorismate pathways. The heterologous biosynthetic system was also designed to include a proofreading enzyme (YbtT) thought responsible for editing the biosynthetic process and removing any mis-primed or incorrect steps in product formation.

The option of exogenously feeding the salicylate starting precursor for Ybt formation allows the potential for compound diversification using an approach termed precursor-directed biosynthesis in which alternative starting units are supplied. Molecular variation then offers the opportunity for altered function which, in this case, means the potential for altered metal-binding properties.

Precursor-directed biosynthesis was utilized to generate five new Ybt analogs. The biosynthetic process was altered to improve analog formation by removing the editing mechanism (encoded by YbtT) that would normally accompany native compound generation. Metabolic engineering was then applied to assist with both precursor transport and endogenous provision. By doing so, the highest producing analog system was compared to the original Ybt compound in the ability to bind gold with a unique chelation pattern observed as a result of analog molecular variation.

To generate analogs of Ybt, different precursors were fed to cultures instead of the native salicylate starter unit and production cultures, compound isolation, and analysis (using native Ybt as a reference standard) were completed as described herein. To improve Ybt and analog production levels, the fadL gene from E. coli was PCR-amplified and introduced to plasmid pCDFDuet-1. The new plasmid was then introduced to the cellular system (described herein) capable of producing Ybt and associated analogs and production attempts commenced as described. The inclusion of FadL helped improve Ybt and analog compound production, presumably by enhancing aromatic precursor transport through the bacterial cell.

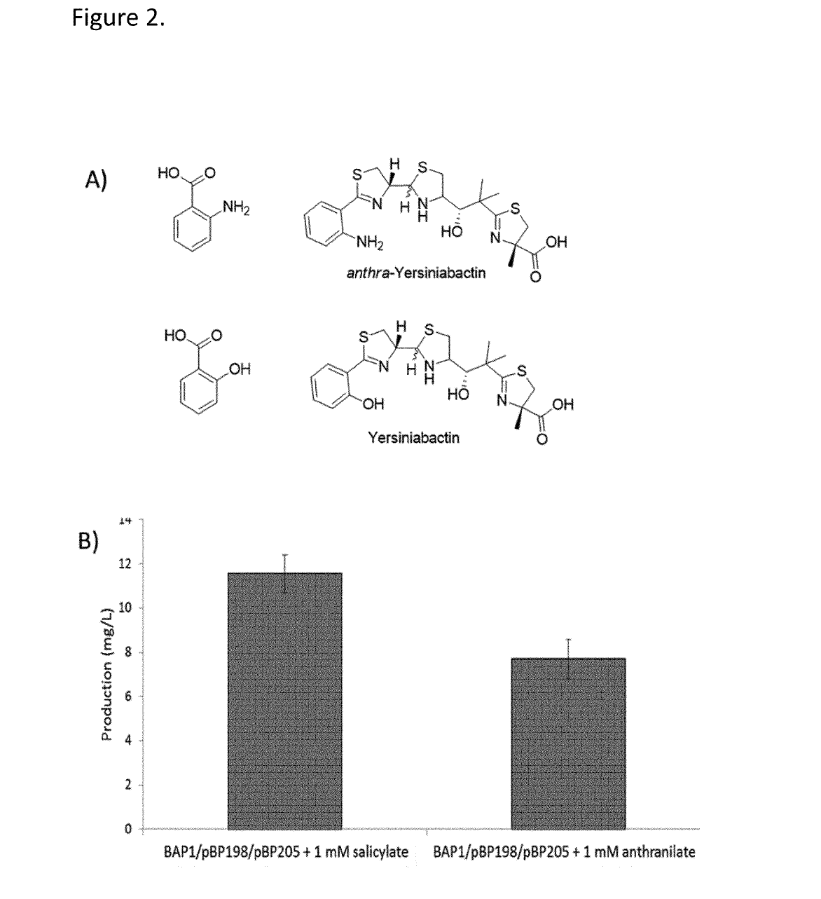

Precursor-directed biosynthesis is used to generate new Ybt analogs with the potential for improved or altered metal binding capabilities. We demonstrated the capability of altering the Ybt molecular structure through the application of precursor-directed biosynthesis. As demonstrated in FIG. 2, the Ybt pathway has shown flexibility in accepting alternative starter units. This flexibility offers the potential to direct new analogs towards specific applications. For example, the analogs selectively remove metal from wastewater samples. Both Ybt and its analogs can be dedicated to specific heavy metal removal. Furthermore, the engineering approaches are also expected be applicable to analog formation as the metabolic engineering approaches, in particular, are dedicated to maximizing the metabolic support mechanisms required for biosynthesis. Representative numbered analogs made using this processes can be seen in FIG. 3.

Materials and Methods.

Strains and plasmids. E. coli strain BAP1 F-ompT hsdSB (rB-mB-) gal dcm (DE3) ΔprpRBCD::T7prom-sfp, T7prom-prpE was used for all experiments. Plasmids containing the genes for Ybt biosynthesis as well as ybtT were provided on individual expression plasmids as described previously. Standard molecular biology protocols were then used to couple these individual genes on multicystronic expression plasmids. pBP198 (carbenicillin resistant and derived from pET21c [Novagen, Milwaukee, Wis.]) contained (in order) HMWP2 and ybtU, with each gene under the control of its own T7 promoter. Likewise, pBP205 (kanamycin resistant and derived from pET28a) contained ybtE followed by HMWP1, with each gene under individual T7 promoters. An additional plasmid, pBP200, contained ybtT under a T7 promoter on a chloramphenicol-resistant plasmid derived from pGZ119EH.

In vivo gene expression and biosynthesis. Strains BAP1/pBP198/pBP205 and BAP1/pBP198/pBP205/pBP200 were used for Ybt biosynthesis with all plasmids or plasmid combinations introduced to BAP1 via electroporation. All cultures used Luria-Bertani (LB) broth media and contained 100 μg of carbenicillin/ml, 50 μg of kanamycin/ml, and 34 μg of chloramphenicol/ml where needed. Cultures (typically 5 to 10 ml) were inoculated 3% (vol/vol) with a previous starter culture, and growth was carried out at 37° C. on a rotary shaker (250 rpm) to an optical density at 600 nm (OD600) between 0.6 and 0.8.

Cultures were then cooled at 20° C. for 10 min (minutes). At this point, 75 μM isopropyl-β-d-thiogalactopyranoside (IPTG) was added together with 1 mM salicylate. Alternatively, the irp9 gene from Yersinia enterocolitica was introduced to the cellular system to enable the endogenous production of salicylate and eliminate the need to feed this precursor to the cultures. The culture was then incubated between 12 and 30 h (hours) at either 13 or 22° C. on a rotary shaker (200 rpm).

For analysis, samples were centrifuged and 5 mM FeCl₃ was then added to the resultant supernatant. The supernatant was then extracted with ethyl acetate for liquid chromatography-mass spectrometry (LC-MS) analysis. Samples extracted were done so twice with an equal volume of ethyl acetate each time. These samples were dried and also submitted for LC-MS analysis. The gradient used for the LC-MS was a linear method from 2 to 98% acetonitrile (balance water) with Ybt-Fe³⁺ observed by MS at about 60% acetonitrile. The fragmentation pattern for Ybt-Fe³⁺ was also determined. Finally, samples taken experimentally were compared by retention times and fragmentation patterns to an authentic sample of Ybt-Fe³⁺. Negative controls included BAP1/pBP198, BAP1/pBP205, and BAP1/pBP198/pBP205 without added salicylate. Cell pellets were sonicated, clarified, and analyzed by sodium dodecyl sulfate-polyacrylamide gel electrophoresis to examine the extent of gene expression for both positive and negative controls.

High cell density fed-batch fermentation for yersiniabactin purification and quantification. The fermentation procedure derives from a similar effort used for complex polyketide production (Pfeifer et al., Appl. Environ. Microbiol. 68: 3287-92, 2002). Briefly, the fermentation medium (termed F1 medium) contained KH₂PO₄ at 1.5 g/liter, K₂HPO₄ at 4.34 g/liter, (NH₄)₂SO₄ at 0.4 g/liter, MgSO₄ at 150.5 mg/liter, glucose at 5 g/liter, trace metal solution at 1.25 ml/liter, and vitamin solution at 1.25 ml/liter. The feed medium contained (NH₄)₂SO₄ at 110 g/liter, MgSO₄ at 3.9 g/liter, glucose at 430 g/liter; trace metal solution at 10 ml/liter; vitamin solution at 10 ml/liter. The trace metals solution consisted of FeCl₃.6H₂O at 27 g/liter, ZnCl₂.4H₂O at 2 g/liter, CaCl₂.6H₂O at 2 g/liter, Na₂MoO₄.2H₂O at 2 g/liter, CuSO₄.5H₂O at 1.9 g/liter, H₃BO₃ at 0.5 g/liter, and concentrated HCl at 100 ml/liter. The vitamin solution consisted of riboflavin at 0.42 g/liter, pantothenic acid at 5.4 g/liter, niacin at 6 g/liter, pyridoxine at 1.4 g/liter, biotin at 0.06 g/liter, and folic acid at 0.04 g/liter.

Fed-batch aerated fermentations were conducted by use of an Applikon 3 L Biobundle system (Applikon Inc., Foster City, Calif.). A starter culture of BAP1/pBP198/pBP205 was grown in 1.5 ml of LB medium (with 100 mg of carbenicillin/liter and 50 mg of kanamycin/liter). After reaching late exponential phase at 37° C. and 250 rpm, the culture was centrifuged and resuspended in 50 ml of LB (at 100-mg of carbenicillin/liter and 50 mg of kanamycin/liter). The culture grew overnight at 30° C. and 200 rpm to stationary phase, was centrifuged, and was resuspended in 20 ml of phosphate-buffered saline for inoculation into the 3-liter vessel containing 2 liters of F1 medium. Growth was conducted at 37° C. with pH maintained throughout the experiment at 7.1 with 1M H₂SO₄ and concentrated NH₄OH. Aeration was maintained at 2.8 liters/min with agitation controlled at 600 to 900 rpm to maintain dissolved oxygen over 50% of air saturation. The fermentation apparatus including a salt solution [KH₂PO₄, K₂HPO₄, and (NH₄)₂SO₄] was autoclaved, whereas the additional components (MgSO₄, glucose, trace metals, and vitamins) were filter sterilized and added aseptically prior to inoculation along with carbenicillin at 150 mg/liter and kanamycin at 75 mg/liter. The feed medium was also filter sterilized. Once the glucose was exhausted from the starting medium (as indicated by a sudden decrease in the oxygen requirement of the culture), the temperature was reduced to 22° C., and IPTG (75 μM) and salicylate (0.160 g/liter) were added. At that point a peristaltic pump started to deliver 0.1 ml of the feed medium/min, and samples were typically taken twice daily thereafter.

Initially, a final fermentation broth was extracted with ethyl acetate (2 liters, twice). The extract was dried, resuspended in water and 10% acetonitrile, and loaded onto a preparatory high-performance liquid chromatography (HPLC) instrument. A 10 to 100% acetonitrile (balance water) gradient at an 8-ml/min flow rate was used to obtain pure Ybt (isolated as an Fe³⁺ chelate). Due to Ybt acid sensitivity, no trifluoroacetic acid was used in the HPLC purification process.

The Ybt fractions eluted at 75% acetonitrile, and these fractions were pooled, dried, and confirmed to contain Ybt-Fe³⁺ by MS. The remaining purified product was resuspended in water and quantified at 385 nm by using the known extinction coefficient (6=2884) for Ybt-Fe³⁺. This initial purified batch of Ybt-Fe³⁺ was then used to generate a calibration curve to quantify production from subsequent fermentation time point samples that were first clarified before directly loading the fermentation broth onto a preparatory HPLC instrument for analysis.

All culture media, sample preparation, and analytical components were purchased from Fisher Chemical (Pittsburgh, Pa.). PCR primers were obtained from Eurofins Genomics (Huntsville, Ala.). Precursors and HAuCl₄ were purchased from Sigma-Aldrich (St. Louis, Mo.).

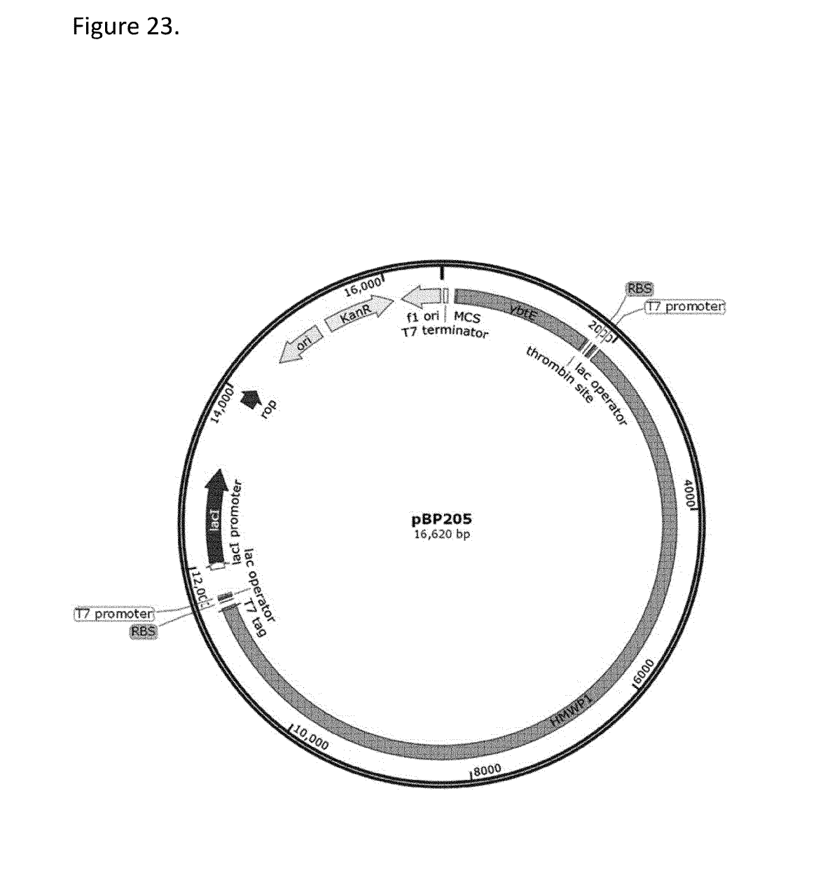

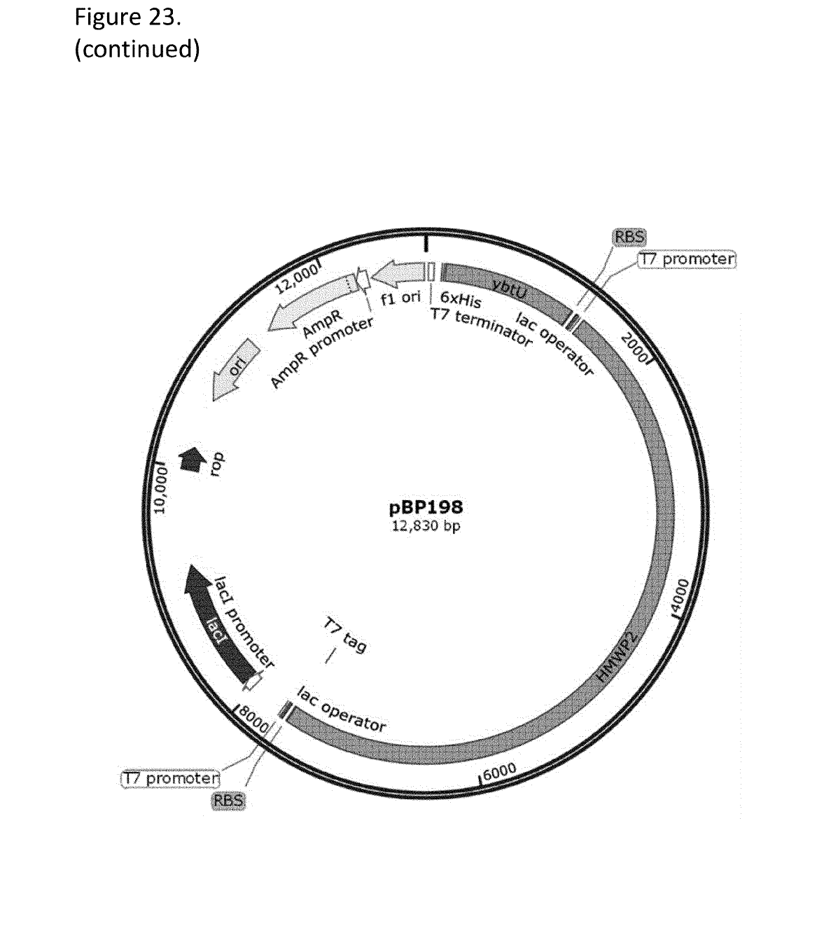

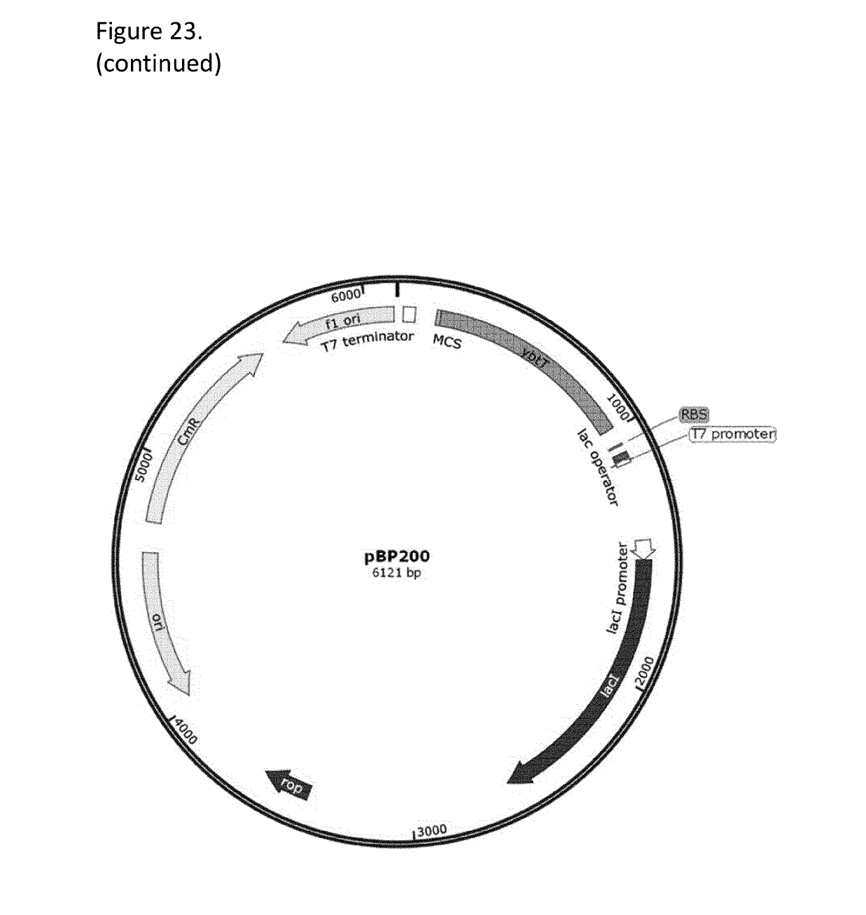

Strain and Plasmid Construction. Strains, plasmids, and PCR primers used in this example are presented in FIG. 23 and Tables 1 and 2. Plasmid cloning was completed using DH5α; whereas, Ybt production was completed in strain BAP1. The fadL, trpD, and trpE genes used during over-expression analysis were isolated from E. coli strain MG1655. Once isolated with the FadL-F and FadL-R primers, the 1.7 kb fadL PCR product was digested with the NdeI and XhoI restriction enzymes and cloned into similarly digested pCDFDuet-1 or pET28a vectors to generate pCDF-fadL or pET28-fadL, respectively. The trpD and trpE genes were amplified and cloned into the NdeI/XhoI and EcoRI/NotI sites of pCDFDuet-1 to construct pCDF-trpDE. BAP1::ΔfadL was generated using the lamda Red gene deletion protocol. Briefly, the pKD3 chloramphenicol acetyltransferase (cat) gene was PCR-amplified using primers pKD3cat-F and pKD3cat-F, double-digested with MfeI/KpnI and cloned into the similarly-digested pET28-fadL (resulting in an internally-disrupted fadL gene). The fadL-cat construct was PCR-amplified with the FadL-KO-F and FadL-KO-R primers and used to perform gene deletion within BAP1. Single or double mutations were introduced into pCDF-fadL by using the QuikChange Site-Directed Mutagenesis Kit (Stratagene). Primers FADLt379c_c380g and FADLt379c_c380g_antisense or FADLc311a_antisense and FADLc311a were designed to substitute serine 100 to arginine or alanine 77 to glutamic acid, respectively. The mutations were verified by nucleotide sequencing.

TABLE 1
Strains and plasmids used in this example.

| Strain | Genotype |
|---|---|
| BAP1 | F-ompT hsdS_{B}(r_{B}⁻ m_{B}⁻) gal dcm (DE3) ΔprpRBCD (sfp) |
| BAP1::ΔfadL | F-ompT hsdS_{B}(r_{B}⁻ m_{B}⁻) gal dcm (DE3) ΔprpRBCD (sfp) |
| | ΔfadL |
| Plasmids | Description |
| pCDF-fadL | pCDFDuet-1 containing fadL from MG1655, Strep^{r} |
| pCDF- | pCDFDuet-1 containing fadL mutant (alanine 77 to |
| fadL::A77E | glutamic acid) |
| pCDF- | pCDFDuet-1 containing fadL mutant (serine 100 to |
| fadL::S100R | arginine) |
| pCDF- | pCDFDuet-1 containing fadL double mutant |
| fadL::A77E, | |
| S100R | |
| pCDF-trpDE | pCDFDuet-1 containing trpD and E genes from MG1655 |
| pET28-fadL | pET28a containing fadL from MG1655, Kan^{r} |
| pCDFDuet-1- | pCDFDuet-1 containing irp9 from Yersinia enterocolitica |
| irp9 | (ATCC 9610), Strep^{r} |
| pBP198 | pET21c containing HMWP2 and ybtU each with their |
| | own T7 promoter, Amp^{r} |
| pBP205 | pET28a containing ybtE and HMWP1 each with their |
| | own T7 promoter, Kan^{r} |
| pBP200 | pGZ119EH containing ybtT under a T7 promoter, Cm^{r} |

TABLE 2
Primers used in this example.

| Primer Name | Sequence |
|---|---|
| FadL-F | AATCATATGGTCATGAGCCAGAAAACC (NdeI) (SEQ ID |
| | NO: 11) |
| FadL-R | AATCTCGAGTCAGAACGCGTAGTTAAAGTT (XhoI)) (SEQ |
| | ID NO: 12) |
| FadL-KO-F | ATGGTCATGAGCCAGAAAACC) (SEQ ID NO: 13) |
| FadL-KO-R | TCAGAACGCGTAGTTAAAGTT) (SEQ ID NO: 14) |
| pKD3cat-F | ATTCAATTGGTGTAGGCTGGAGCTGCTTC (MfeI) (SEQ ID |
| | NO: 15) |
| pKD3cat-R | ATTGGTACCATGGGAATTAGCCATGGTCC (KpnI) (SEQ ID |
| | NO: 16) |
| FADLt379c_ | TGGGGCGCTTCTATTACCCGTAACTATGGTCTGGCTAC |
| c380g | (SEQ ID NO: 17) |
| FADLt379c_ | GTAGCCAGACCATAGTTACGGGTAATAGAAGCGCCCCA |
| c380g_antisense | (SEQ ID NO: 18) |
| FADLc311a_ | TGTTCGGAACCCATTCCGTAGGCGCGATG (SEQ ID |
| antisense | NO: 19) |
| FADLc311a | CATCGCGCCTACGGAATGGGTTCCGAACA (SEQ ID |
| | NO: 20) |
| TrpD-F | ATTCATATGGCTGACATTCTGCTGCTCGAT (NdeI) (SEQ ID |
| | NO: 21) |
| TrpD-R | ATTCTCGAGTTACCCTCGTGCCGCCAGTGC (XhoI) (SEQ |
| | ID NO: 22) |
| TrpE-F | ATTGAATTCGCAAACACAA AAACCGACTCTC (EcoRI) |
| | (SEQ ID NO: 23) |
| TrpE-R | ATTGCGGCCGCTCAGAA AGTCTCCTGTGCATG (NotI) |
| | (SEQ ID NO: 24) |

Plasmids needed for Ybt (or analog) biosynthesis included pBP198 (pET21c containing genes encoding HMWP2 and YbtU), pBP205 (pET28a containing genes encoding HMWP1 and YbtE), and pBP200 (pGZ119EH containing the gene encoding YbtT). Plasmid pCDFDuet-1-irp9 encodes for a salicylate synthase from Yersinia enterocolitica capable of supporting heterologous E. coli Ybt biosynthesis without the need for exogenous salicylate addition. Plasmids were transformed into BAP1 or BAP1::ΔfadL using electroporation. Following selection with appropriate antibiotics, resulting strain-plasmid combinations were stored as 20% glycerol stocks.

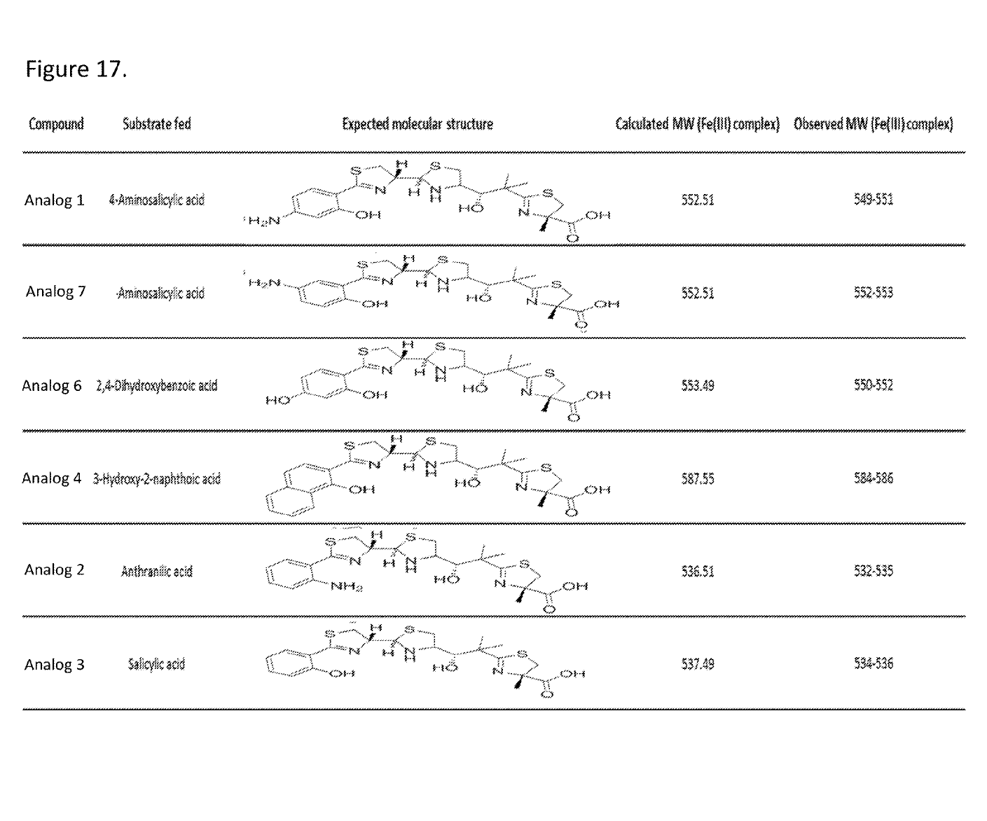

Bacterial Culture and Compound Extraction. Overnight cultures from glycerol stocks were incubated at 37° C. with shaking in lysogeny broth (LB) medium and used to inoculate (1% v/v) 25 mL cultures of M9 minimal medium (per liter: 12.8 g Na₂HPO₄.7H₂O; 6 g Na₂HPO₄; 3 g KH₂PO₄; 0.5 g NaCl; 1 g NH₄Cl; pH adjusted to 7.4 with NaOH) supplemented with glycerol (0.5 wt %) and casamino acids (0.2 wt %). Post-inoculation, cultures were incubated at 22° C. with shaking for two days with induction initiated at an OD₆₀₀ₙₘ of 0.4-0.6 using 100 μM isopropyl β-D-1-thiogalactopyranoside (IPTG). For those cultures requiring exogenous supplementation, 1 mM of specific substrate (FIG. 17) was added 3 hours after induction. Culture plasmid selection was maintained with 100 μg/mL ampicillin, 50 μg/mL kanamycin, 20 μg/mL chloramphenicol, and 50 μg/mL spectinomycin, as needed (the same antibiotic levels were used for solid medium transformation selection). Post-culture, 1 mL acetone was added per 50 mL of medium and incubated for an additional 30 minutes prior to centrifugation to collect the resulting supernatant for extraction. To the supernatant, Fe³⁺ was added (via FeCL₃ addition) at a final concentration of 5 mM followed by ethyl acetate extraction twice with an equal volume of ethyl acetate each time. Extracts were combined, evaporated to dryness under vacuum, and the resulting residue was resuspended in methanol (with the final volume concentrated 10× relative to the original culture volume).

For the FadL transport analysis, the BAP1::ΔfadL and BAP1 strains both contained the required Ybt biosynthetic pathway (i.e., pBP198/pBP205); however, the former strain was tested with the background expression plasmid and plasmids encoding FadL mutants and wild-type FadL. The strains were cultured to an OD₆₀₀ₙₘ of 0.4-0.6 at 37° C. with shaking prior to induction with IPTG (100 μM), followed by continued growth for 5 hr at 30° C. with shaking. After this time, the culture cells were washed twice with M9 medium and resuspended in M9 medium supplemented with 0.5% glycerol and 1 mM salicylate for additional culture at 30° C. with shaking for 2 hr. At which point, 5 mM FeCl₃ was added prior to supernatant LC-MS analysis.

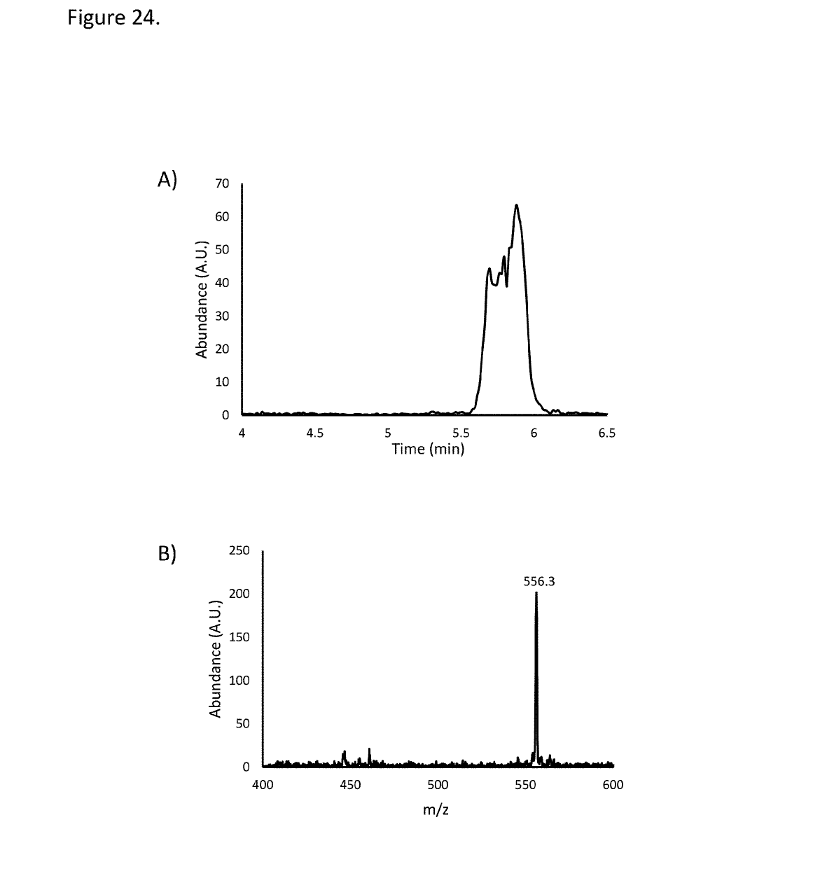

To generate a ¹³C-labeled Ybt compound to be used as an internal standard during subsequent analyses, a 5 mL culture containing strain BAP1/pBP198/pBP200/pBP205/pCDF-1-irp9 was grown overnight to inoculate (1% v/v) a 5 mL culture grown to an OD₆₀₀ₙₘ of 0.4-0.6 using LB medium at 37° C. with shaking. Next, a 12.5 mL culture was inoculated (1% v/v) in M63 medium (per liter: 2 g (NH₄)₂SO₄; 13.6 g KH₂PO₄; 0.5 mg FeSO₄.7H₂O; 0.5 g NaCl; pH adjusted to 7.4 with KOH) containing 2% ¹³C3-D-glucose and 10 mg/mL thiamine. After incubation for 12 hours at 30° C. with shaking, cells were removed by centrifugation and a frozen stock of supernatant was kept for use as an internal standard. Isotopic labeling was confirmed by LC-MS (FIG. 24).

HPLC and LC-MS Analysis. Purified Ybt product was achieved using an Agilent 1200 preparatory HPLC system equipped with a Waters C18, 5 μm, 300 Å, 150×3.9 mm ID column. In this setting, a 1 mL/minute flow rate was used together with a 10-100% acetonitrile (balance water) gradient over 15 minutes. Collected fractions containing product were quantified at 385 nm using the known extinction coefficient for Ybt-Fe³⁺ (ε=2,884). After addition of the internal standard supernatant (10% of final sample volume [with this level maintained for all samples analyzed]), purified Ybt was then used to produce a calibration curve for compound quantification via LC-MS analysis using an API 3000 Triple Quad LC-MS with a Turbo Ion Spray source (PE Sciex) coupled with a Shimadzu Prominence LC system. In the absence of pure analog standards, the Ybt calibration curve was used for all quantification purposes. The MS analyses were conducted in positive ion mode, and chromatography was performed on a Waters X Terra C18 column (5 μm, 2.1 mm×250 mm). After an injection of 4 μL of final extract, conditions for LC-MS were 10-100% acetonitrile (balance water) gradient over 15 minutes at a flow rate of 0.2 mL/minute. Production levels were determined relative to a calibration curve using purified Ybt and a ¹³C-labeled Ybt internal standard.

Gold-binding Assay To test Ybt or analog gold binding capability, HAuCl₄ was used to generate a solution of 1 mM Au(III) in de-ionized water. To this solution, crude extract of Ybt or analog (from samples prepared without FeCl₃ addition) was added at a final concentration of 2-5 μM. After incubation for 2 hours at 22° C., the solution was analyzed by LC-MS.

Statistical Evaluation. Data presented were generated from three independent experiments. Error bars represent standard deviation values. All statistical significance comparisons between groups were performed using a one-way ANOVA with Dunnett post-tests.

Results and Discussion. Precursor-directed Biosynthesis of Ybt Analogs. The exogenous addition of the Ybt starting unit opens the opportunity for directed manipulation of the final compound via precursor feeding. Several commercially-available starter unit variants were selected as outlined in FIG. 17. The alternate precursors retained an aromatic structure similar to the original salicylate substrate, yet variation in benzyl functional groups or overall ring structure allowed the assessment of biosynthetic flexibility in generating new analogs.

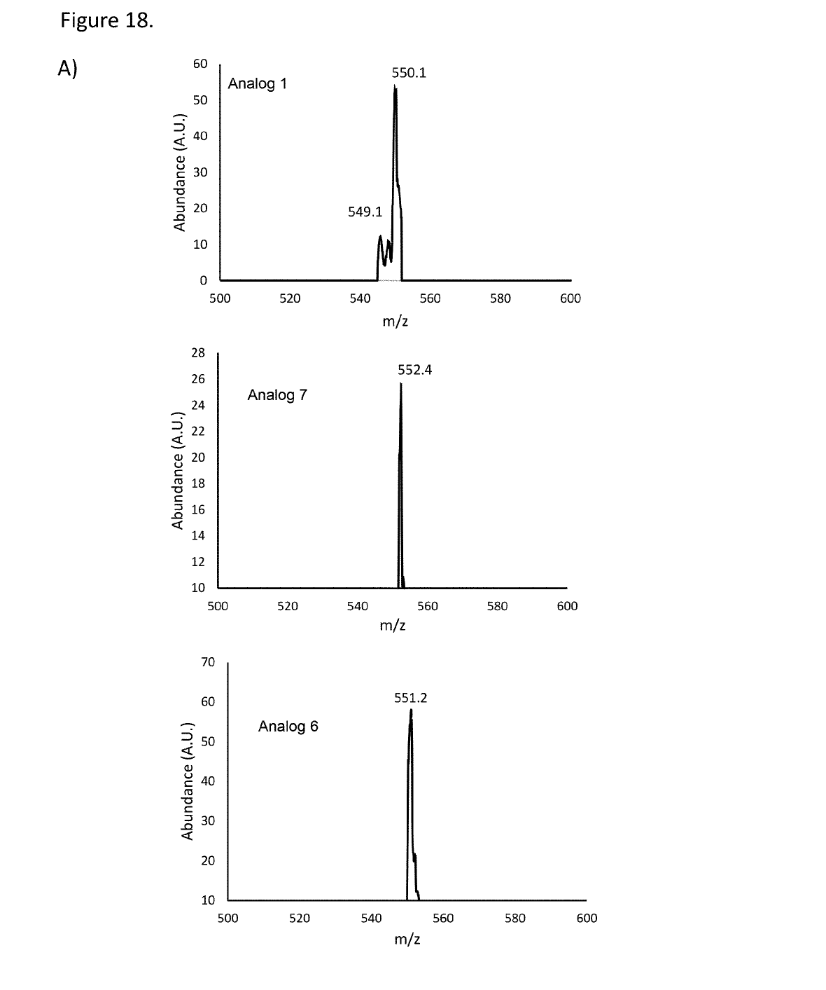

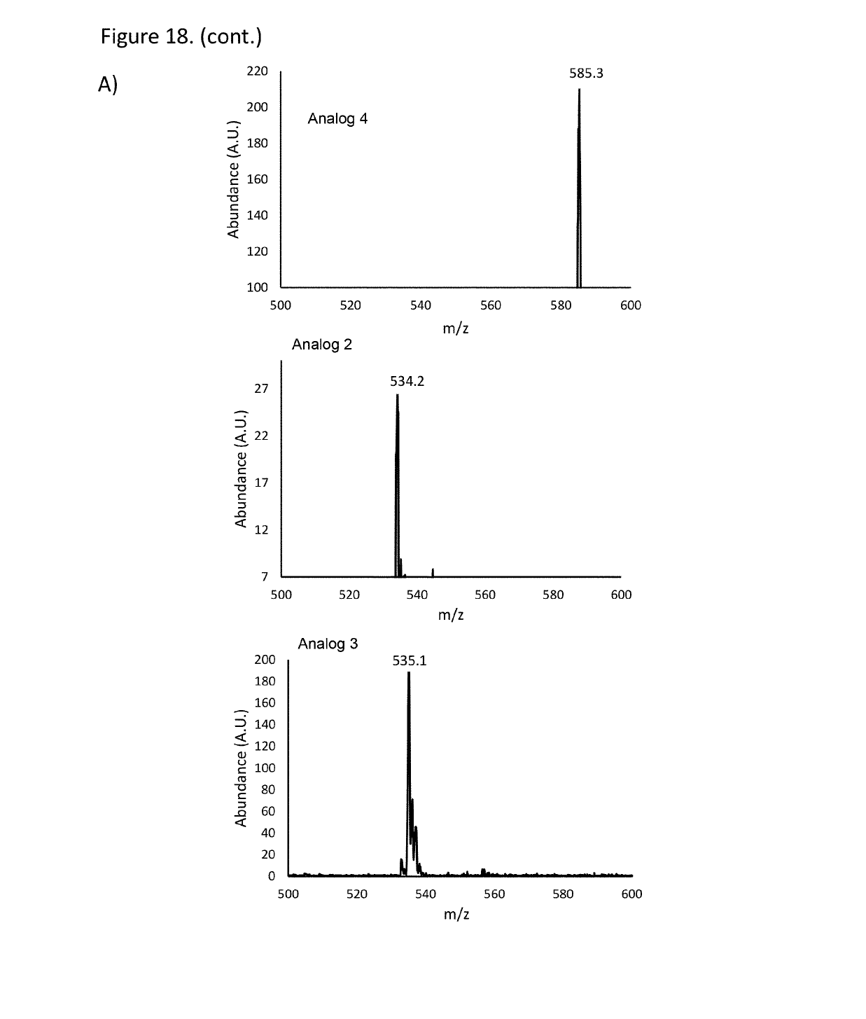

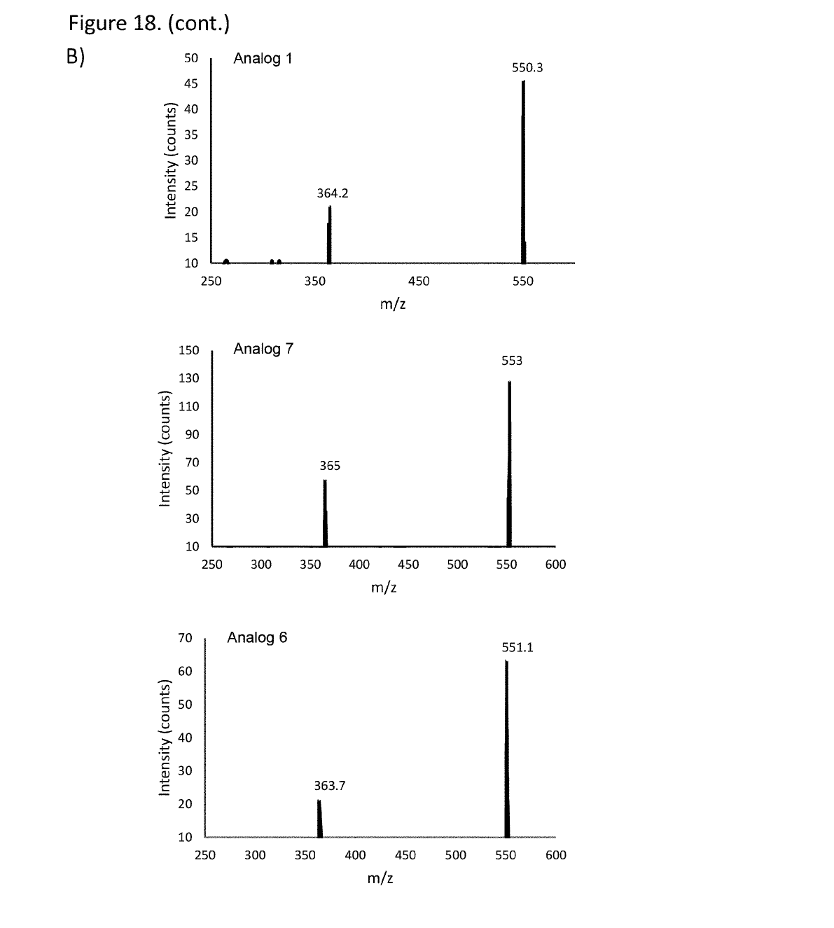

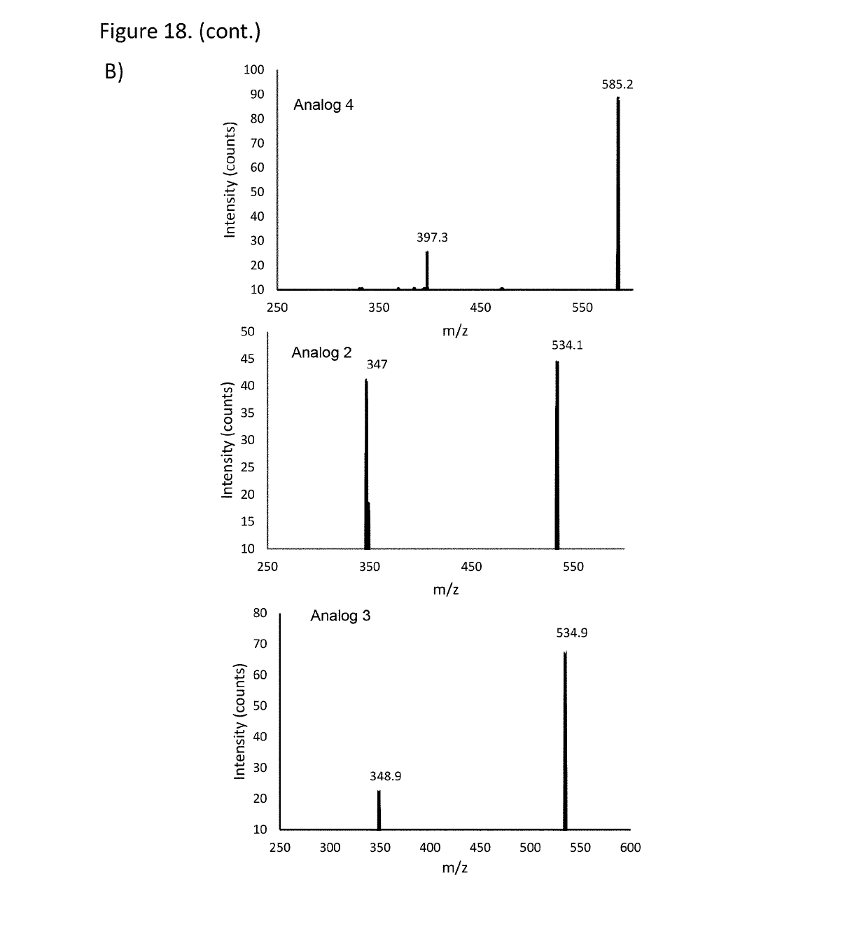

As outlined in FIG. 18, five new Ybt analogs resulted as profiled by LC-MS and LC-MS/MS. Compounds all retained Fe³⁺ binding capability. Each also demonstrated a fragmentation pattern typical of a neutral loss from the native Ybt compound.

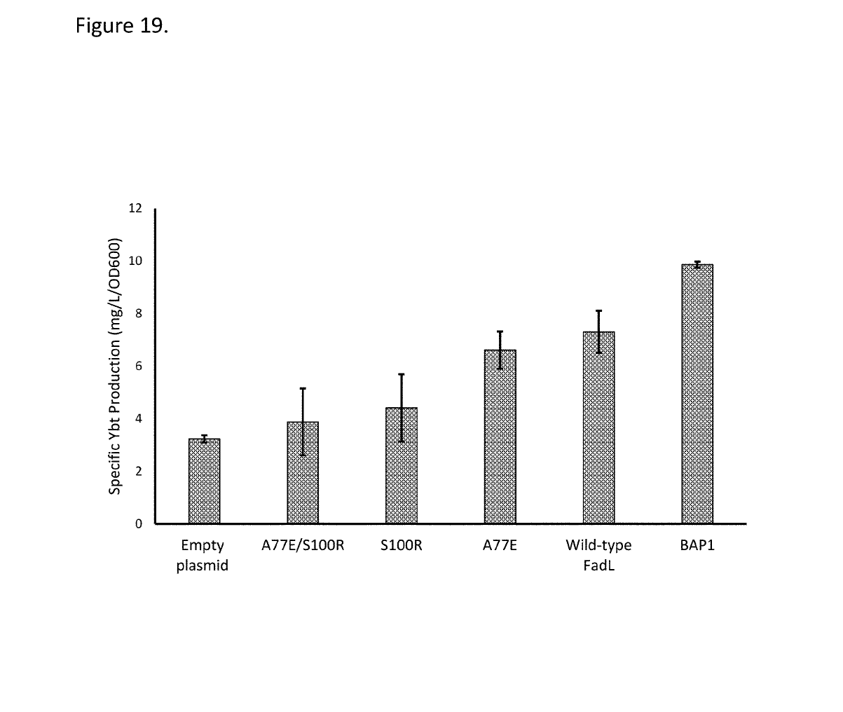

FadL Influence On Intracellular Availability of Exogenously Fed Precursors. One potential caveat of precursor-directed biosynthesis is the limitation to compound transport posed by the cellular membranes of E. coli. To test this issue and provide a potential solution, a series of FadL variants were generated. The FadL protein family is involved in the cellular transport of fatty acid and other hydrophobic compounds, including aromatic species. We reasoned this mechanism might also aid the transport of the precursors tested in this example. To that end, a series of FadL mutants, including single and double mutations known to inactivate the protein, were tested in the context of Ybt biosynthesis using exogenously fed salicylate. When tested in a fadL knockout mutant of E. coli, decreased Ybt production was observed with reduced compound formation more pronounced for an S100R mutation (FIG. 19). Interestingly, Ybt production was still observed without FadL, indicating some level of passive transport (or alternative active transport) of the salicylate precursor through the E. coli cell.

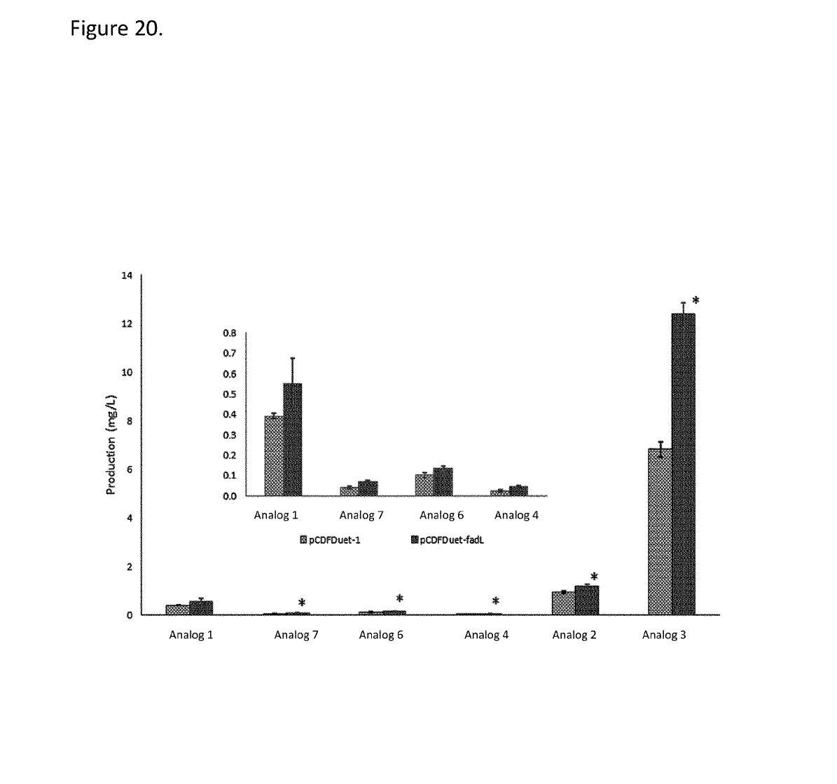

Next, a fadL over-expression construct was tested for impact upon Ybt and analogs formed via precursor-directed biosynthesis (FIG. 20). In every case, the inclusion of FadL over-production improved Ybt or analog formation. The results further support the transport capabilities of FadL in relation to the precursors fed in the directed biosynthesis of Ybt-based compounds. Importantly, consistent improvement across all analogs demonstrates a degree of breadth in the capability of transporting different precursors.

However, FIG. 20 and Table 3 also indicate reduced titer values as analogs are generated compared to the native Ybt compound. Though the provision of enhanced levels of FadL may help address transport issues associated with the precursors fed in analog production attempts, there still remains the question of efficient incorporation and processing of non-native substrates during biosynthesis. The identification of novel analogs demonstrates the plasticity of the biosynthetic pathway, but the associated lowered titers indicate the tradeoff in terms of production capability relative to the native Ybt compound.

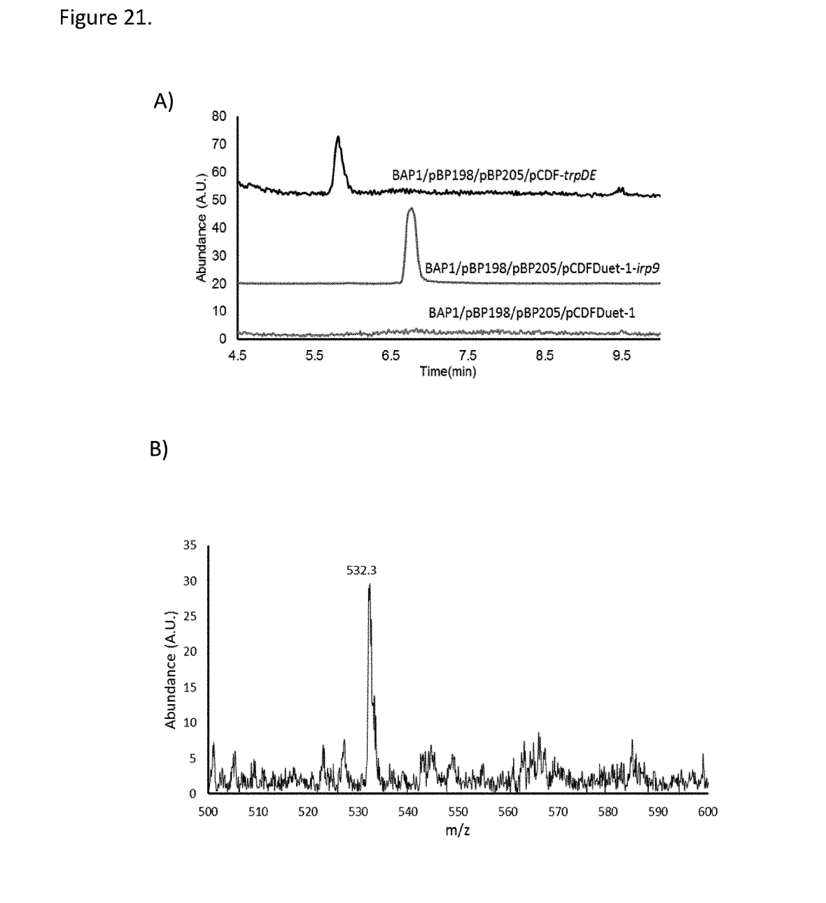

Metabolic Engineering Precursor Support for the Anthranilate Analog. The E. coli metabolic pathway for L-tryptophan includes the conversion of chorismate to anthranilate via the activity of TrpE and TrpD. Hence, in an effort to improve endogenous levels (i.e., originating from within the organism) of anthranilate for analog production, trpD and E were over-expressed using pCDF-trpDE. A similar approach was utilized to provide endogenous salicylate via irp9 over-expression. Both approaches are provided in FIG. 21 with successful Ybt and anthranilate analog formation achieved.

TABLE 3
effect on Ybt and analog production with and without YbtT.

| Compound | With YbtT | Without YbtT |
|---|---|---|
| Analog 1 | 0.07 ± 0.01 | 0.39 ± 0.01 |
| Analog 7 | 0.03 ± 0.01 | 0.04 ± 0.01 |
| Analog 6 | 0.04 ± 0.01 | 0.10 ± 0.01 |
| Analog 4 | 0.01 ± 0.003 | 0.02 ± 0.01 |
| Analog 2 | 0.44 ± 0.08 | 1.2 ± 0.04 |

The use of metabolic engineering to provide precursors for the yersiniabactin pathway consolidates complete biosynthesis and demonstrates that the approach can be extended to an analog compound. As such, the results feature complementarity between chemistry and biology since precursor-directed biosynthesis allows a chemical means of diversification via modified precursors while metabolic engineering offers the potential to provide those same precursors if suitable pathways can be designed. Greater chemical diversity is available with exogenously fed precursors; whereas, engineering endogenous precursor support may eliminate the cost and effort required to chemically generate substrates (though this depends on intracellular precursor support matching that possible through exogenous addition and the lack of any unwanted metabolic burden resulting from pathway engineering).

The metabolic engineering pathway for the anthranilate precursor resulted in a final analog titer of 0.052 mg/L compared to 1.2 mg/L when exogenous substrate feeding was completed. Though analog production was observed during trpDE over-expression, a likely reason for the reduced titer relative to exogenous supplementation is that the remainder of the tryptophan pathway remains intact. Thus, competition will exist for the enhanced anthranilate levels between the analog formation and remaining tryptophan pathway steps. Separately, the engineering step (i.e., trpDE over-expression) to provide enhanced endogenous anthranilate does not account for precursor adenylation, provided by YbtE in the case of salicylate activation for Ybt production. Hence, if YbtE cannot effectively adenylate variant starting precursors, the opportunity for inefficiencies at this step and/or subsequent biosynthetic processing heightens the possibility for reduced final titers. Thus, the flexibility of YbtE to adenylate foreign starter units may be as important in final analog titers as the subsequent steps in final compound formation.

Removal of the YbtT Editing Mechanism and the Effect on Analog Titer. YbtT (encoded on pBP200) is expected to play a role in editing the biosynthesis of native Ybt by removing aberrant compounds from the enzymatic complex during product formation. As a result, YbtT is likely actively limiting the formation of Ybt analogs. To test this possibility, production was compared with and without YbtT (Table 3). For each analog, removal of YbtT resulted in improved titers with increases observed over a range of 1.3- to 5.6-fold. As expected, YbtT removal had a negative impact on Ybt formation. Such a result further supports the editing role of YbtT and allows a means of improving analog formation by better enabling the flexibility of the biosynthetic pathway.

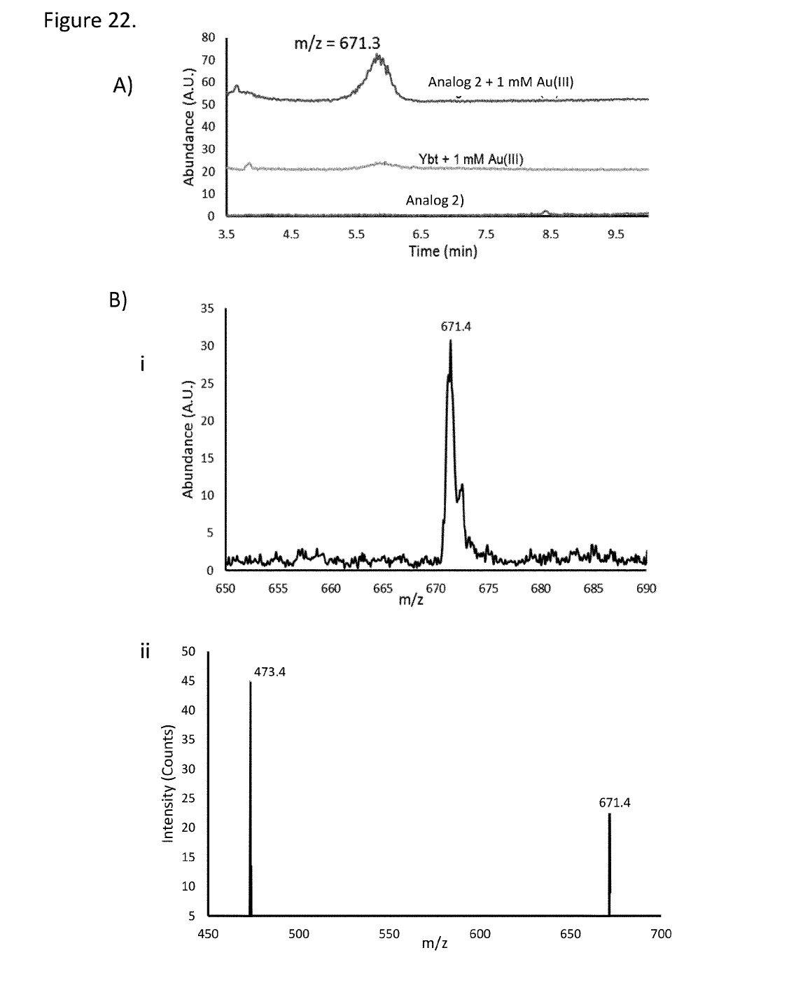

Comparison of Ybt and the Anthranilate Analog in Gold Binding. As an assessment of analog formation, gold binding was compared between the native Ybt compound and the anthranilate analog (chosen because it was the analog with the highest titer). In FIG. 22, LC-MS reveals a clear difference between gold complex formation with the anthranilate analog and the lack of complexation when testing Ybt. These results suggest preferential metal binding can be derived using the precursor-directed diversification strategy presented herein. As such, the approach and engineering strategies provided in this example serve as a basis in future work to be directed at applications such as metal retrieval and re-use across industrial and environmental engineering settings.

Precursor-directed biosynthesis was used to generate five new analogs of the Ybt compound from a heterologous E. coli production platform. Different strategies were then utilized to improve or alter analog formation including biosynthetic editing relaxation and metabolic engineering for precursor transport and provision. Finally, preferential gold binding was observed for an anthranilate analog compound compared to native Ybt.

### Example 2

This example describes methods of making and using Ybt and Ybt analogs.

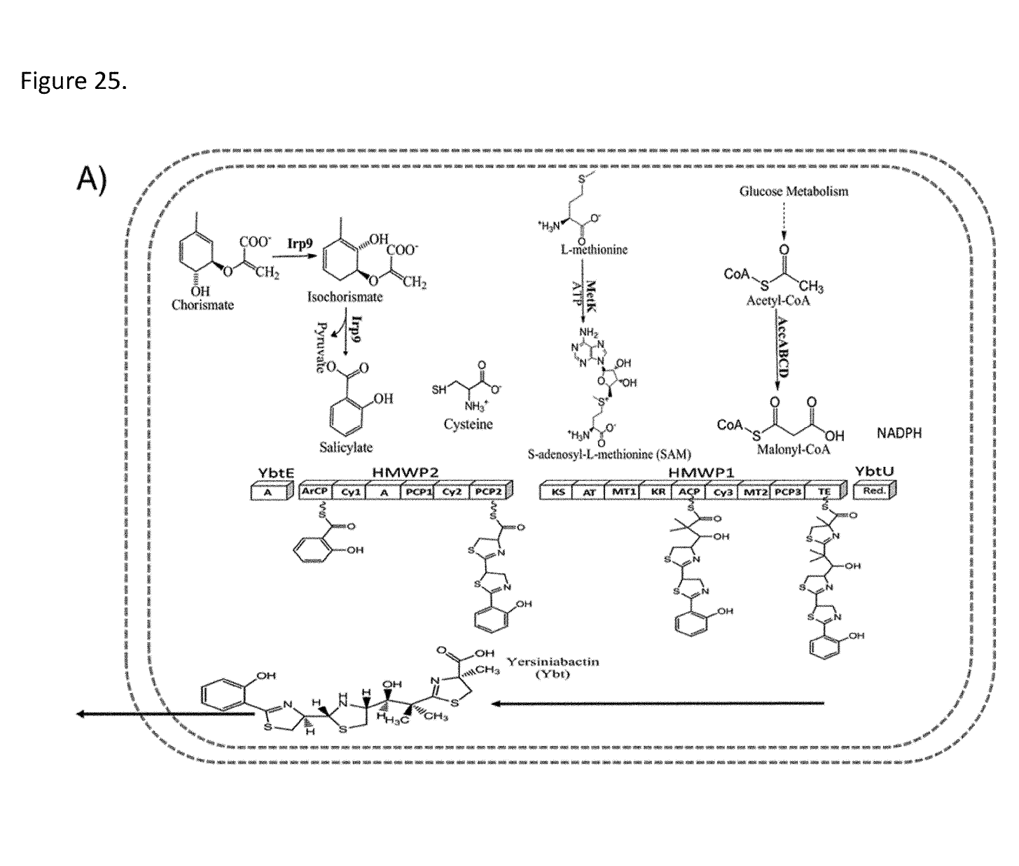

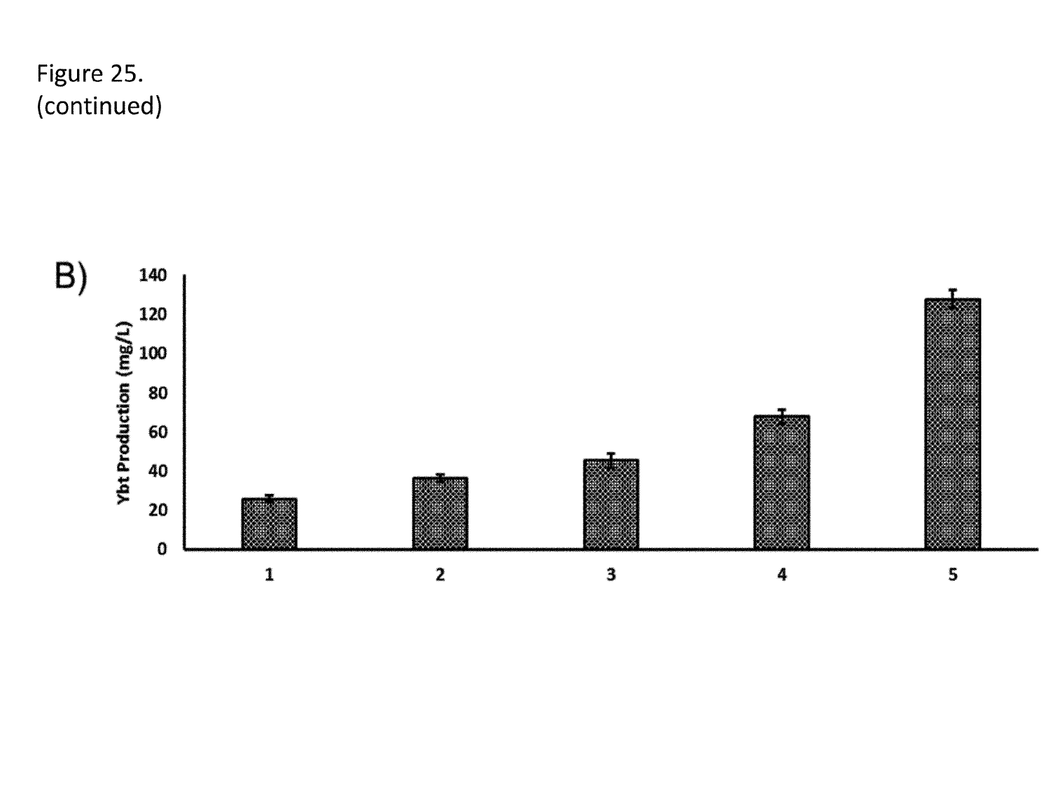

We developed a heterologous production process for Ybt and associated analogs and tested their ability to remove iron (Fe^{III}), copper (Cu^{II}), and gold (Au^{III}) from solid and liquid samples. The heterologous production system consists of the Ybt biosynthetic pathway reconstituted through Escherichia coli (FIG. 25A). By doing so, the danger of working with the native pathogenic source organism is eliminated. Furthermore, the E. coli host offers extensive metabolic and process engineering opportunities to improve and diversify the original biosynthetic process.

We applied metabolic engineering principles to the E. coli heterologous platform that produces Ybt for the purpose of extending production. This goal is tethered to improving the subsequent performance of a packed-bed water treatment model that has been impregnated with the Ybt compound. The system was then used to remove copper from mixed metal aqueous solutions across a range of process parameters to assess and optimize the process, including the potential to readily recover the sequestered metal and regenerate the metal-removal column.

Eventual applications derived from Ybt will be connected to the production source of the compound. Production through E. coli avoids use of the pathogenic original host, but heterologous biosynthesis must sufficiently support subsequent applications. To improve current production metrics, a series of metabolic engineering strategies was enacted (FIG. 25A). The steps highlighted each introduce metabolic engineering to channel carbon to Ybt substrates or coenzymes. Namely, the Irp9, MetK, and ACC enzymes were expected to enhance levels of intracellular salicylate, S-adenosylmethionine, and malonyl-CoA, respectively.

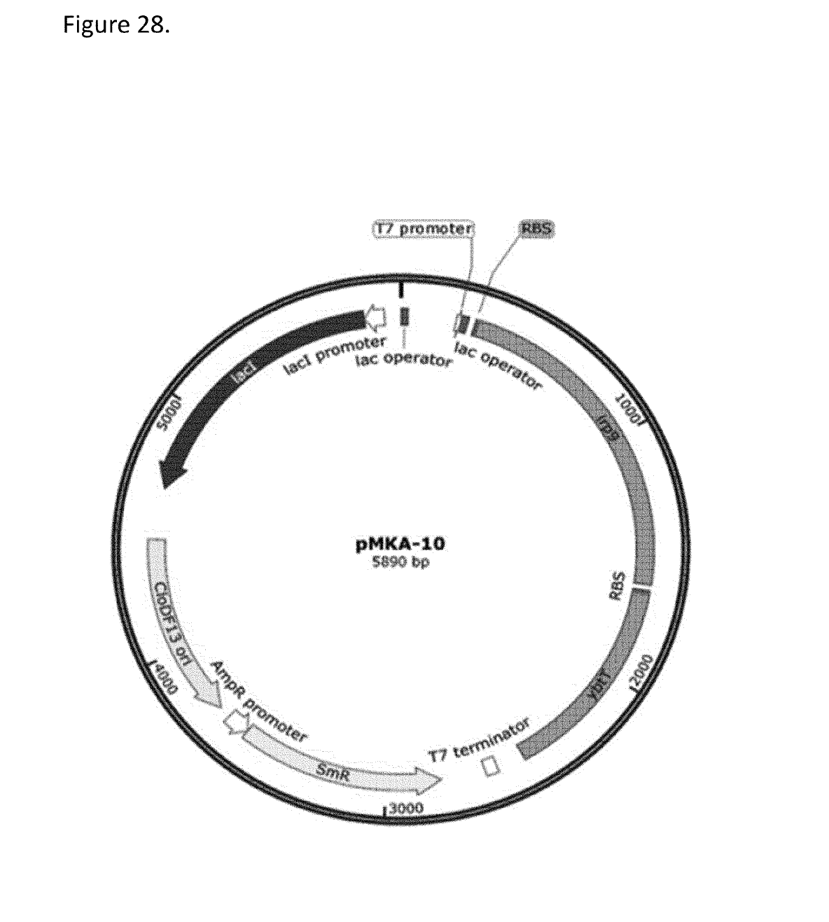

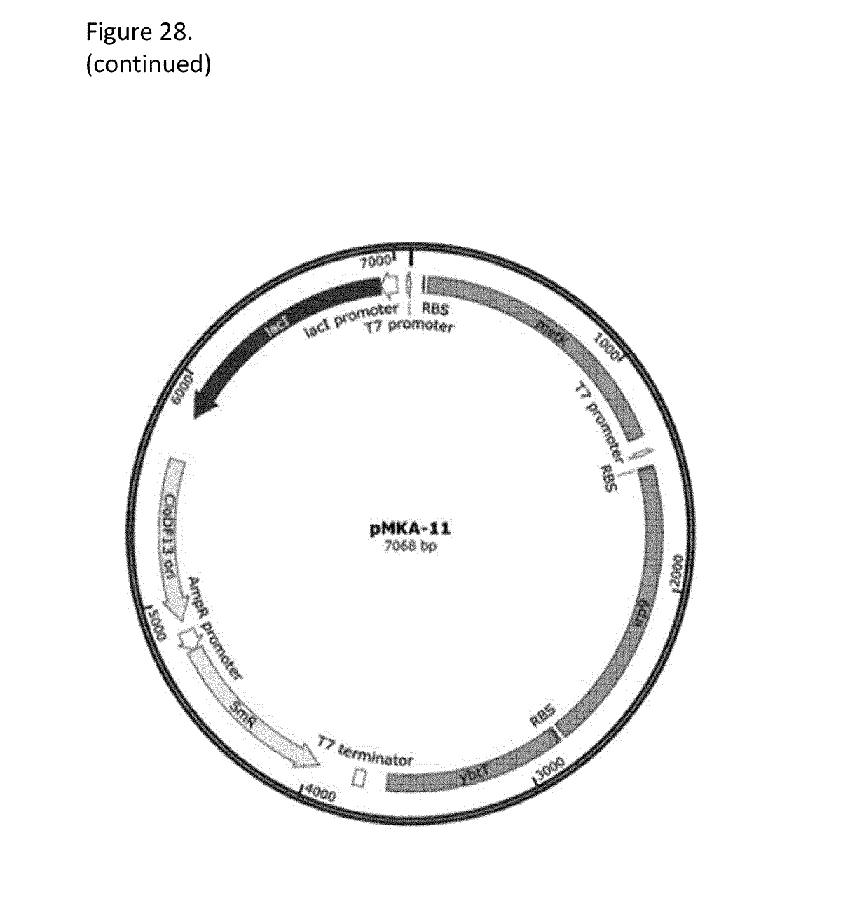

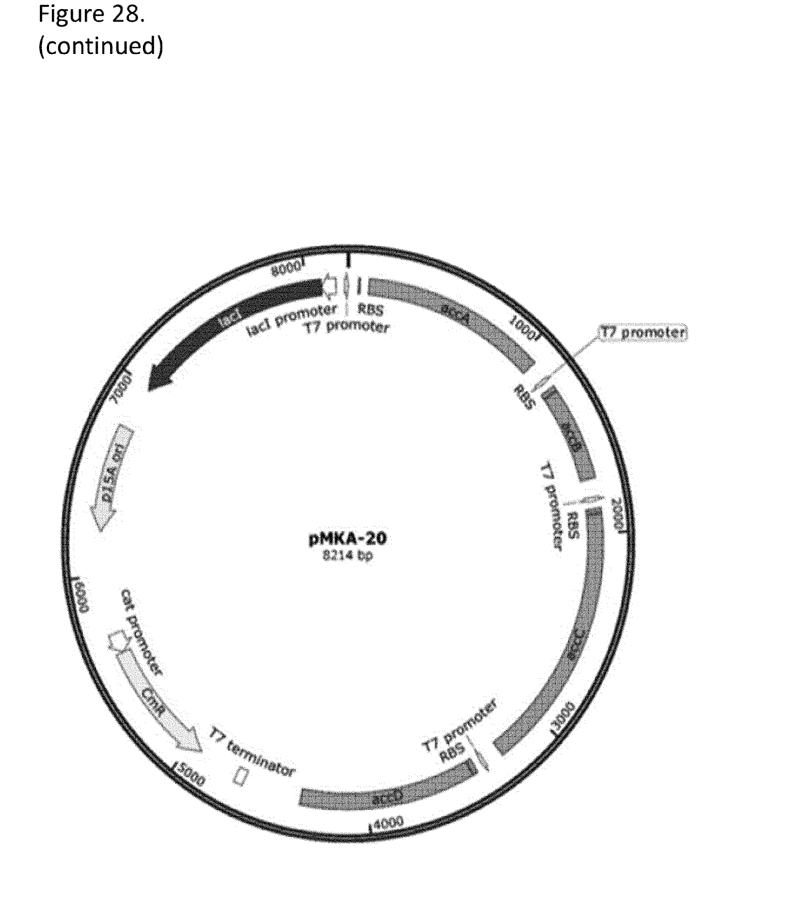

When plasmids designed with the required genes were introduced to E. coli strain BAP1 (engineered to support polyketide and nonribosomal peptide biosynthesis; Tables 1 and 2 and FIG. 28), production levels consistently increased (FIG. 25B). Interestingly, though final Ybt titers improved between 1.5-3 fold with the metabolic engineering steps taken, the exogenous addition of cysteine extended final Ybt levels to ˜120 mg/L in shake flask cultures, approximately 6× the original amount produced. This last result suggests metabolic engineering to increase cysteine levels would also be a successful route to improving final Ybt production.

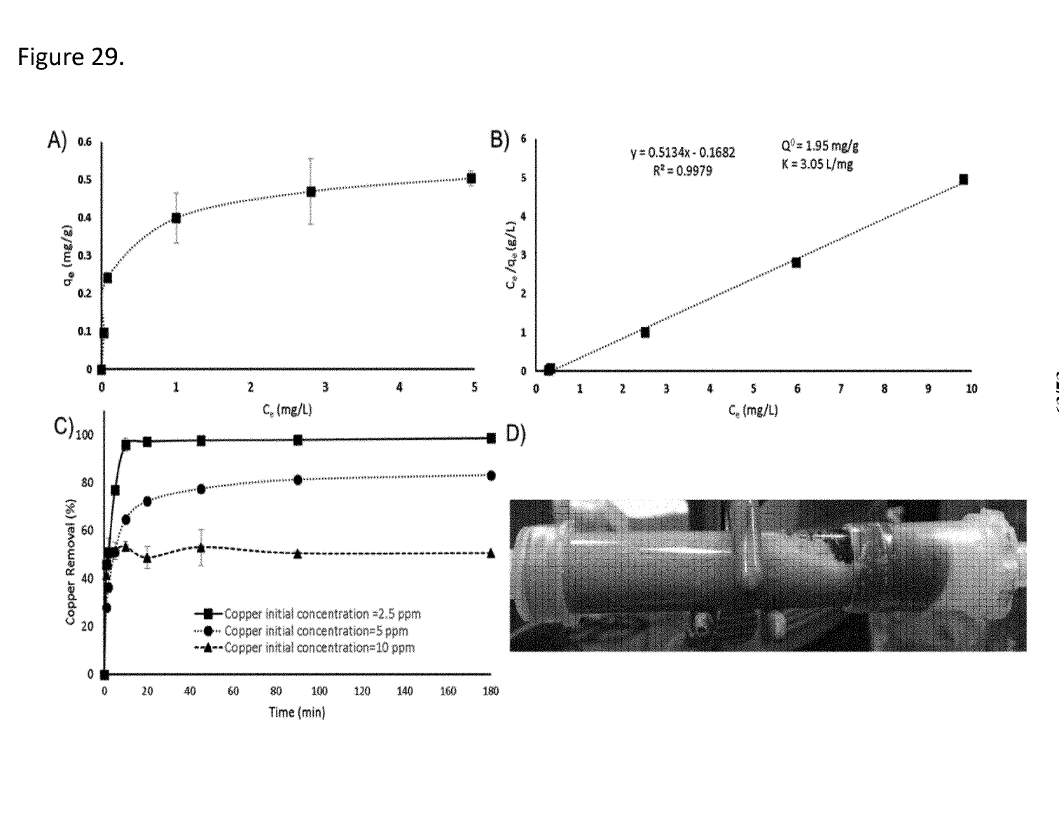

The improved heterologous Ybt system was then used as a source for the development of a packed-bed model to remove metal from water streams (FIG. 29). Ybt was adsorbed to XAD16 resin (chosen due to previous use in natural product in situ capture and henceforth referred to as XAD). The resulting matrix was then loaded into a column and placed within a continuous stream of water infused with metal mixtures. Copper and zinc mixtures were used to test selective copper removal. The mixture would also help assess the selectivity of the Ybt-XAD resin when compared to a common metal scavenging resin, Chelex 100.

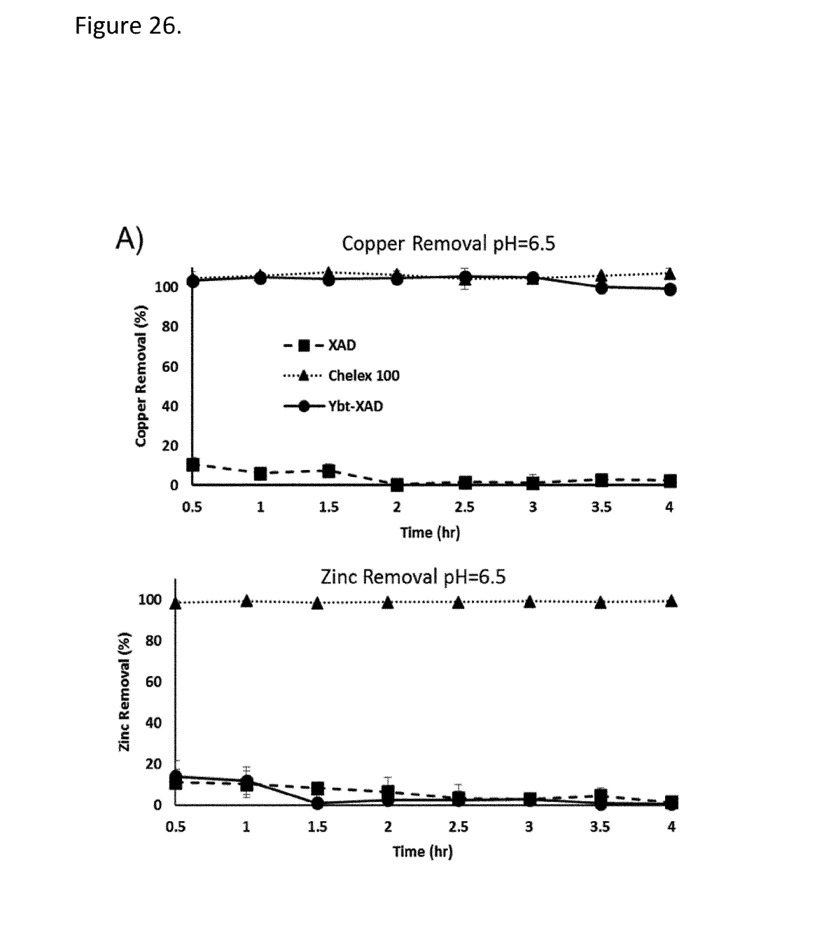

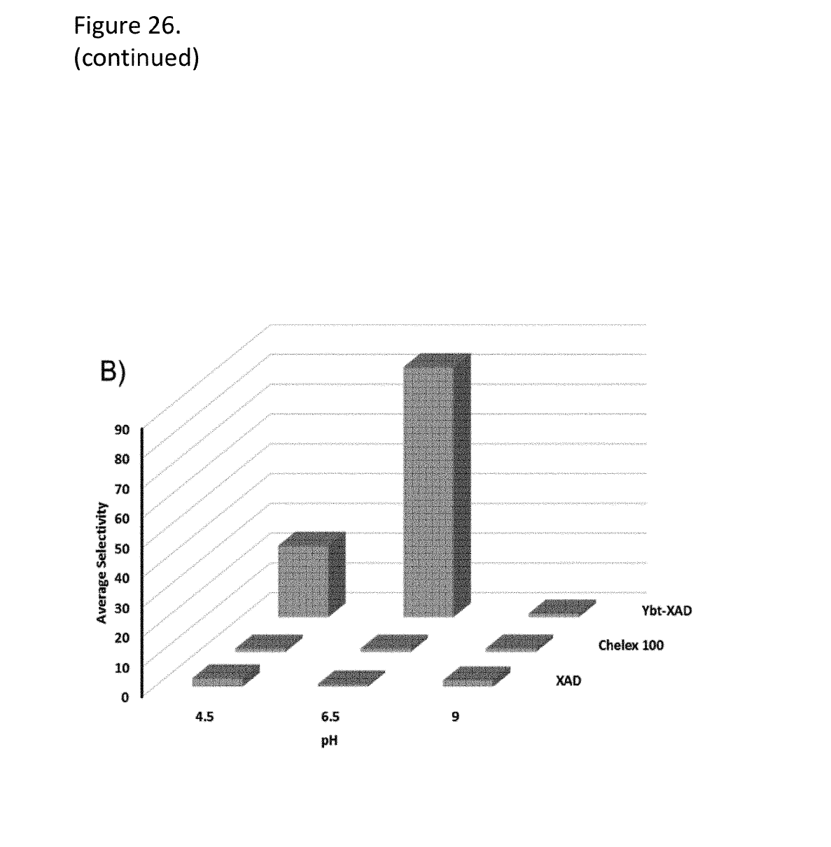

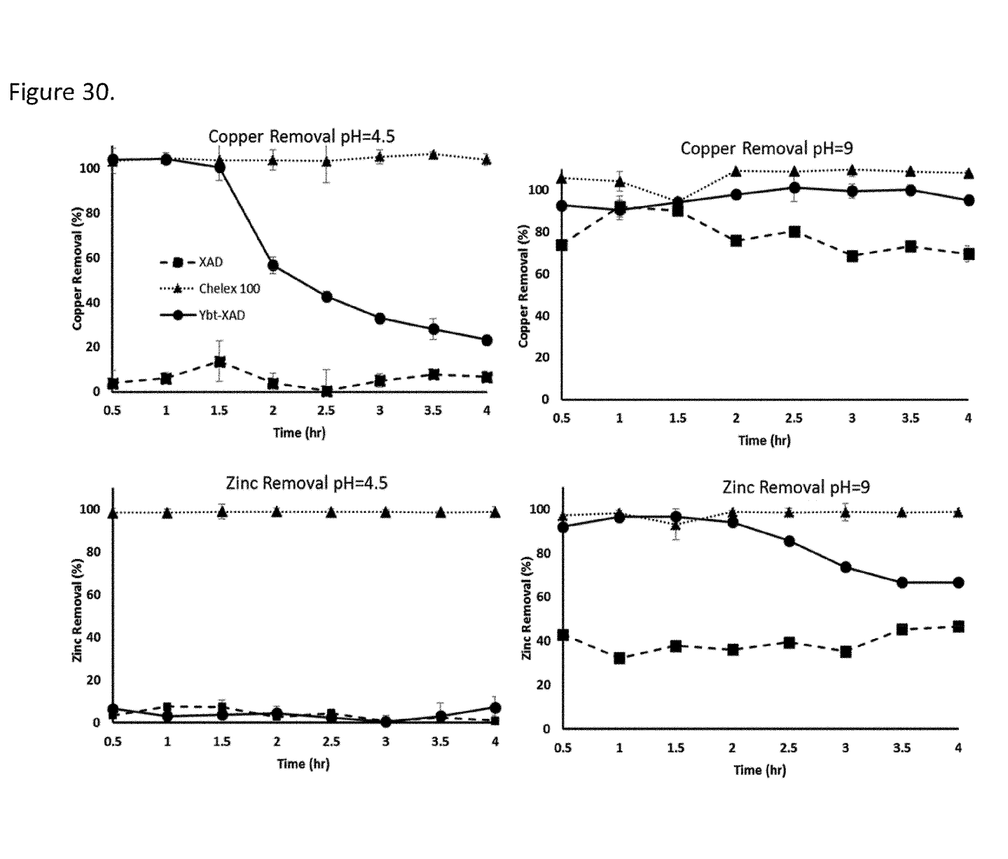

FIG. 26 and FIG. 30 compare the copper removal profiles for Ybt-XAD, XAD, and Chelex 100 across three pH levels (4.5, 6.5, and 9). Ybt-XAD outperforms Chelex 100 for both removal and selectivity at pH 6.5; whereas, performance is compromised for removal, selectivity, or both at the other pH levels tested. The pH levels will clearly affect the charge properties of Ybt and, hence, the coordination chemistry behind metal chelation. The data suggest an optimum of pH 6.5 which is further indicated in FIG. 26B, which summarizes the selectivity of the three samples tested. Of note, Ybt has been implicated in the ability to bind zinc in solution near neutral pH values. However, this is not observed in our fixed bed experiments at pH 6.5, suggesting a Ybt conformational change upon XAD adsorption that disrupts zinc binding as supported by other siderophore studies.

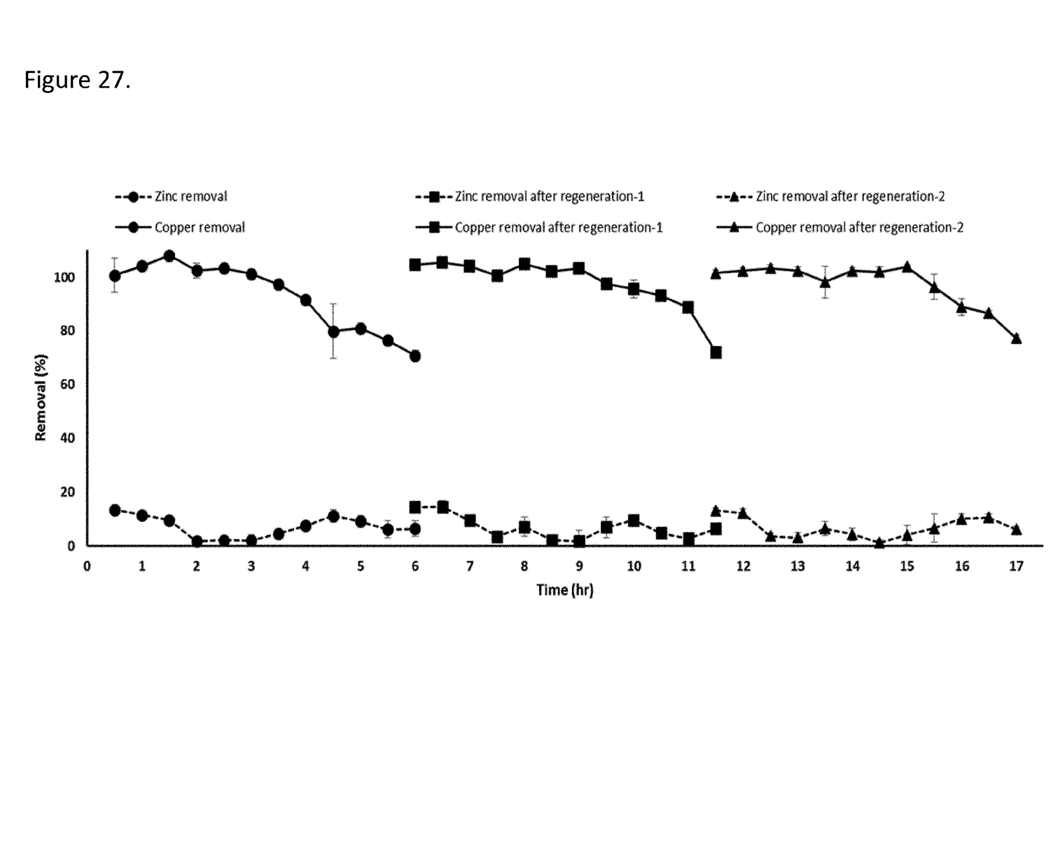

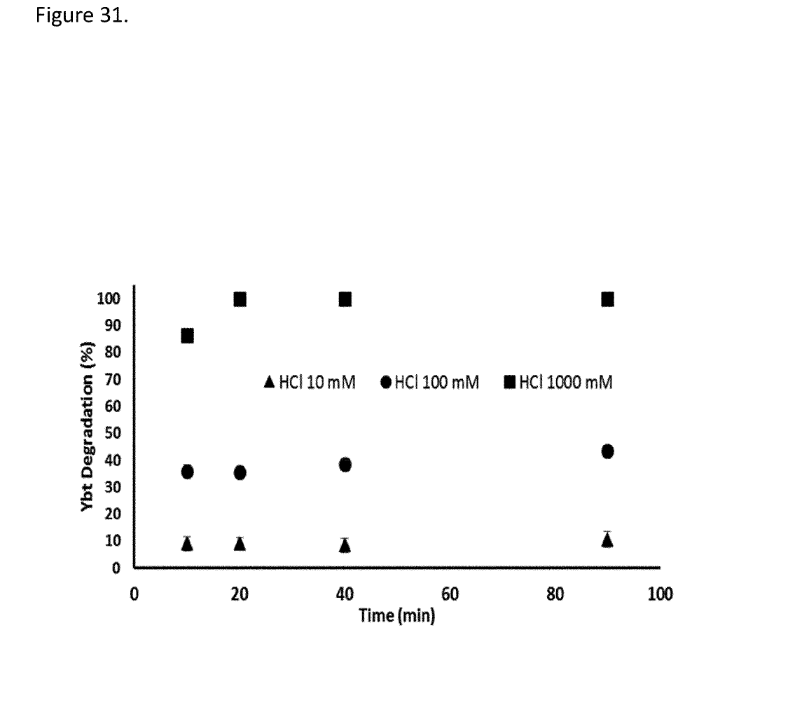

Based upon the pH removal profiles for Ybt-XAD, the column was tested for regeneration (FIG. 27). Because a lower pH showed decreased copper removal over time (FIG. 30), the model configuration was exposed to acidic conditions periodically throughout a time course. To select the degree of pH reduction, the Ybt compound was tested for degradation over log-fold differences in HCl concentrations (FIG. 31). Ybt showed stability at pH 2, thus, this level was utilized during the course of the regeneration experiments. As observed, column saturation effects can be reversed at time points that pH adjustment occurred, allowing both extended column use and the potential to recover bound metal content over a short time frame.

In summary, a multi-disciplinary application of water metal removal derived from natural product biosynthesis is described. Heterologously produced Ybt provided a biosynthetic platform that enabled metabolic and process engineering steps to improve final titers. With improved access to the Ybt compound, a packed-bed continuous water treatment model was used to test selective removal of copper in a metal-recoverable and column-regenerative format with performance metrics linked to the metal-binding properties of Ybt and the unique natural product origins of this compound.

Experimental. Bacterial culture and Ybt-XAD preparation: Overnight 3 mL cultures from glycerol stocks were incubated at 37° C. with shaking in lysogeny broth (LB) medium and used to inoculate (1% v/v) 25 mL cultures of M9 minimal medium (per liter: 12.8 g Na₂HPO₄-7H₂O; 6 g Na₂HPO₄; 3 g KH₂PO₄; 0.5 g NaCl; 1 g NH₄Cl; pH adjusted to 7.4 with NaOH) supplemented with glycerol (0.5 wt %) and casamino acids (0.2 wt %). Post-inoculation, cultures were incubated at 22° C. with shaking for two days with induction initiated at an OD₆₀₀ₙₘ of 0.4-0.6 using 100 mM isopropyl β-D-1-thiogalactopyranoside (IPTG). For those cultures requiring exogenous supplementation, 2 mM of cysteine was added 1 h after induction. Culture plasmid selection was maintained with 100 mg/mL ampicillin, 50 mg/mL kanamycin, 20 mg/mL chloramphenicol, and 50 mg/mL spectinomycin, as needed (the same antibiotic levels were used for solid medium transformation selection). Post-culture, 1 mL acetone was added per 50 mL of medium and incubated for an additional 30 min prior to adding 5 mM FeCl₃ to the culture supernatant, which was then diluted 100× in methanol prior to LC-MS analysis to measure Ybt production.

To modify XAD16 resin with Ybt, the same biosynthetic production procedure was utilized with the following modifications: the bacterial culture size was increased to 500 mL and the post-induction incubation time was increased to four days. XAD16 (activated according to manufacturer instructions) was added to the BAP1/pBP198/pBP205/pMKA-11/pMKA-20 culture on day two (15 gram resin per 500 mL cell culture) post-induction. Upon completion of the four day culture, the resin was filtered and washed with excess Millipore water three times before being resuspended in Millipore water for further tests.

Copper removal experiments: To test copper removal, 10 grams of each resin (XAD, Chelex 100, and Ybt-XAD) were packed into a 2.5×10 cm glass chromatography column (Bio-Rad). The column was equilibrated with 10 bed volumes of Millipore water before loading the metal solution. The metal solution (˜5 ppm zinc and ˜1 ppm copper; pH adjusted with dilute HCl or NaOH) was pumped (LMI Milton Roy dosing pump) into the column with a continuous flow rate of 1 L/hr. Samples were collected every 30 min. The percent copper or zinc removal was calculated using the following formula: [(Cᵢ−C_{f})/Cᵢ]×100, where Cᵢ is the initial copper (or zinc) concentration and C_{f} is the final concentration. Average selectivity was defined as the ratio of average copper removal (%) to the average zinc removal (%) over the course of the experiment.

We introduced Ybt and analog XAD beads to metal-containing samples. Amberlite® XAD-16N beads were washed twice with methanol and twice with water before finally being resuspended in Millipore water. Next, 250 μL of Ybt- or analog-containing crude extract was added to 0.5 g of XAD beads and the final volume adjusted to 10 mL using Millipore water. Mixtures were then incubated at 30° C. overnight prior to washing and resuspending beads in Millipore water.

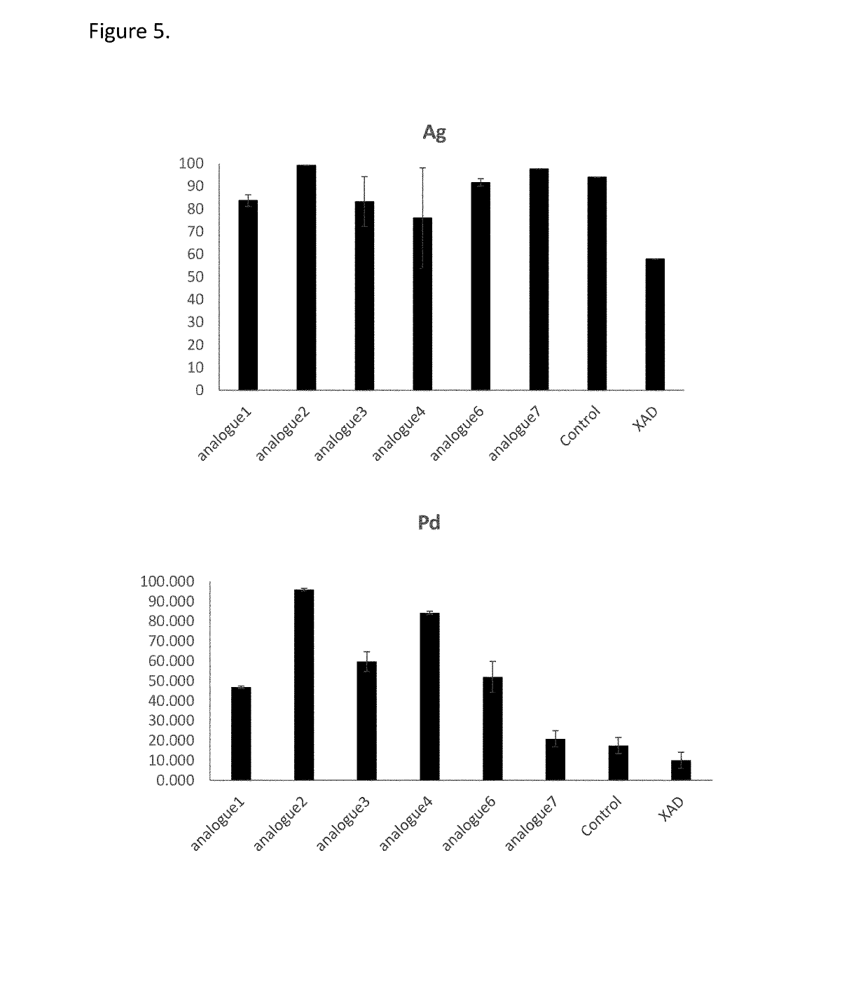

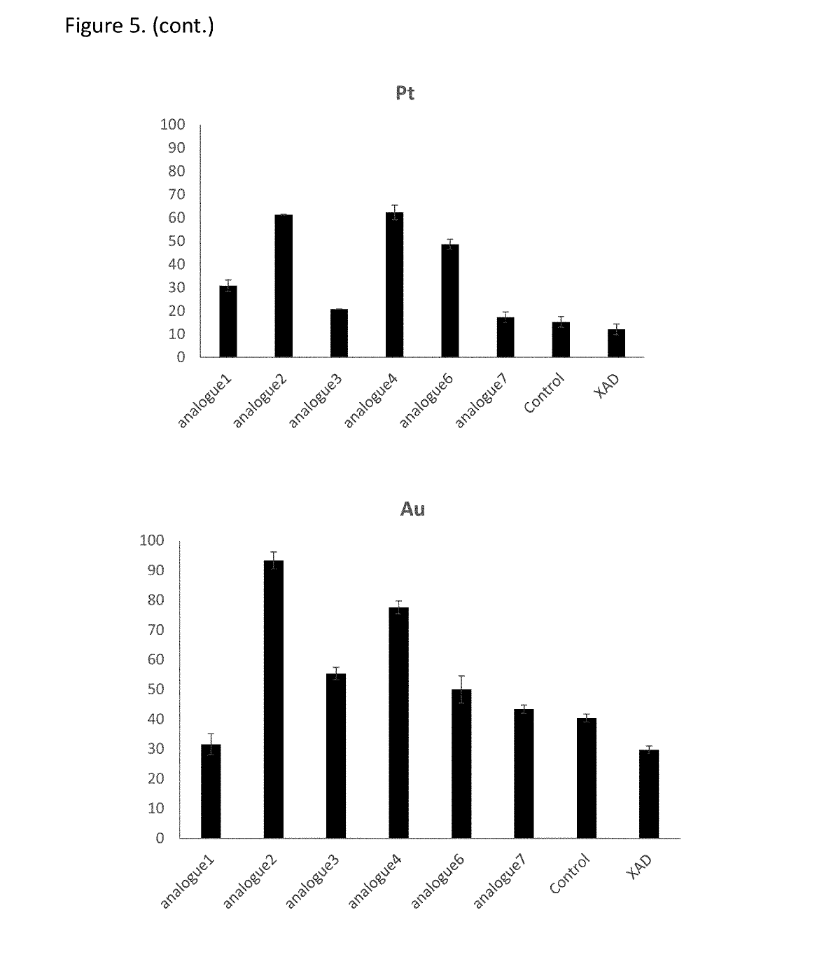

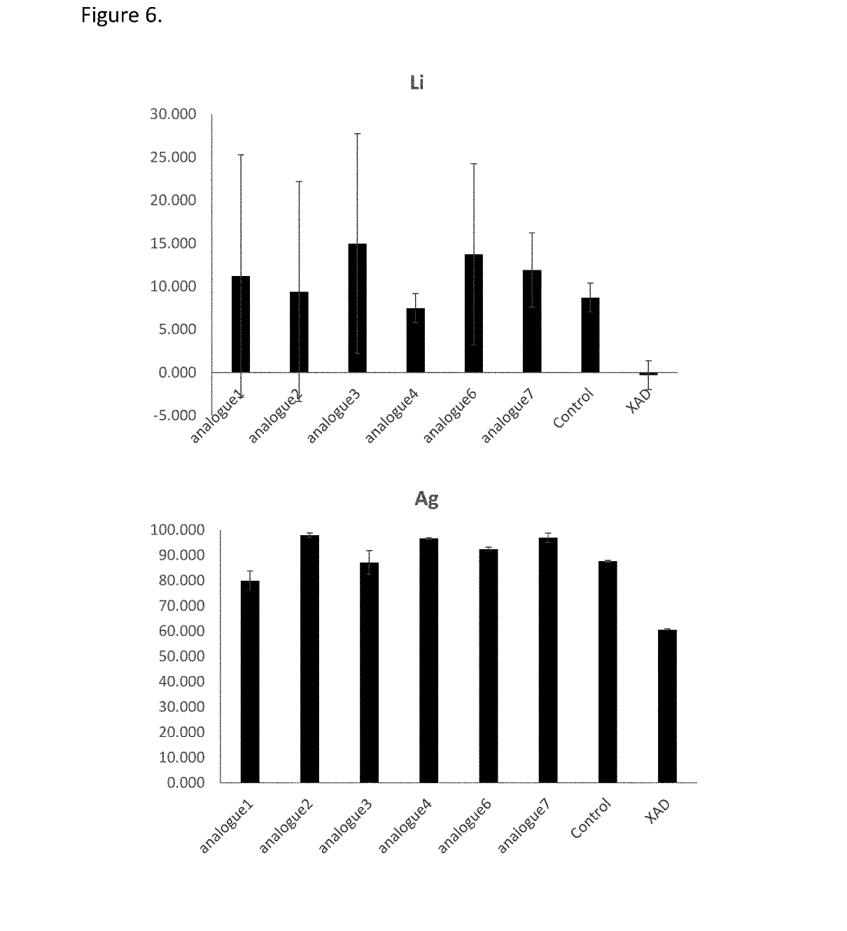

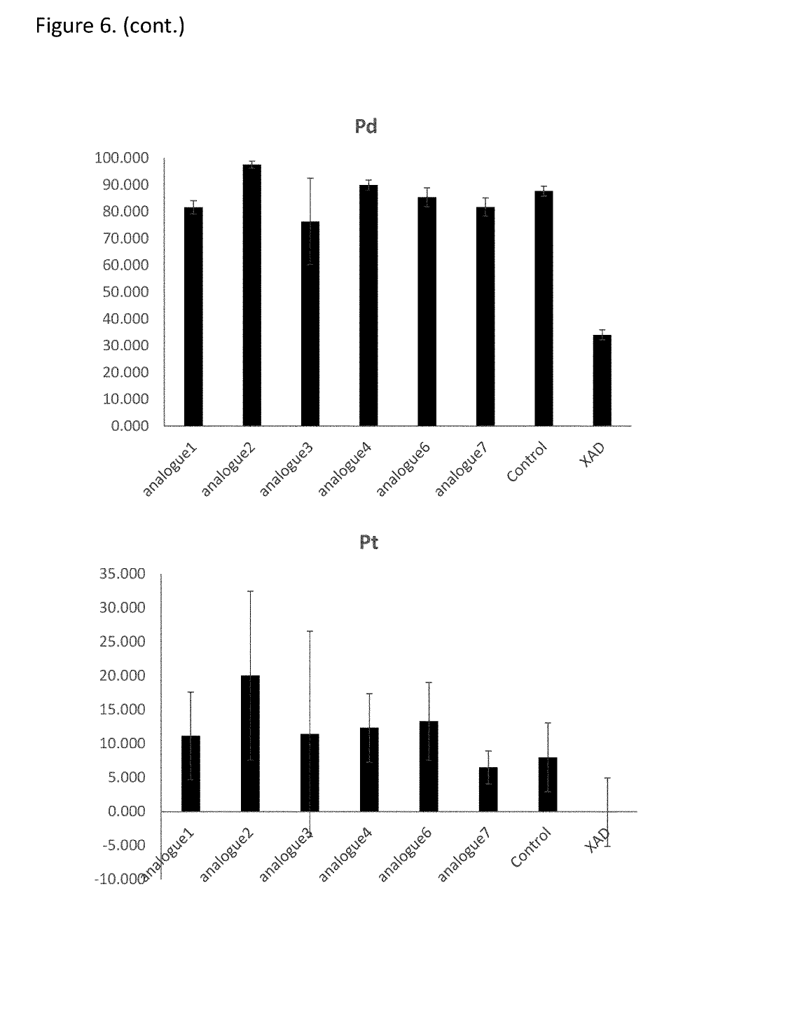

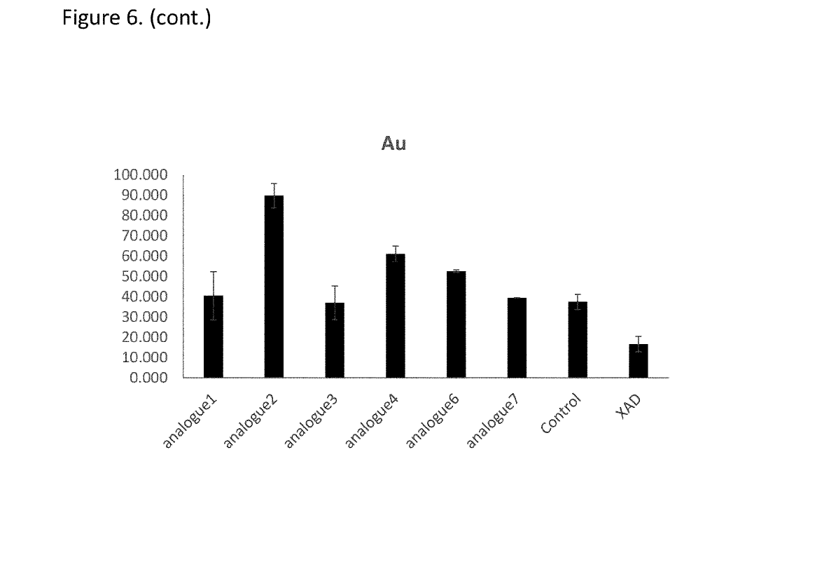

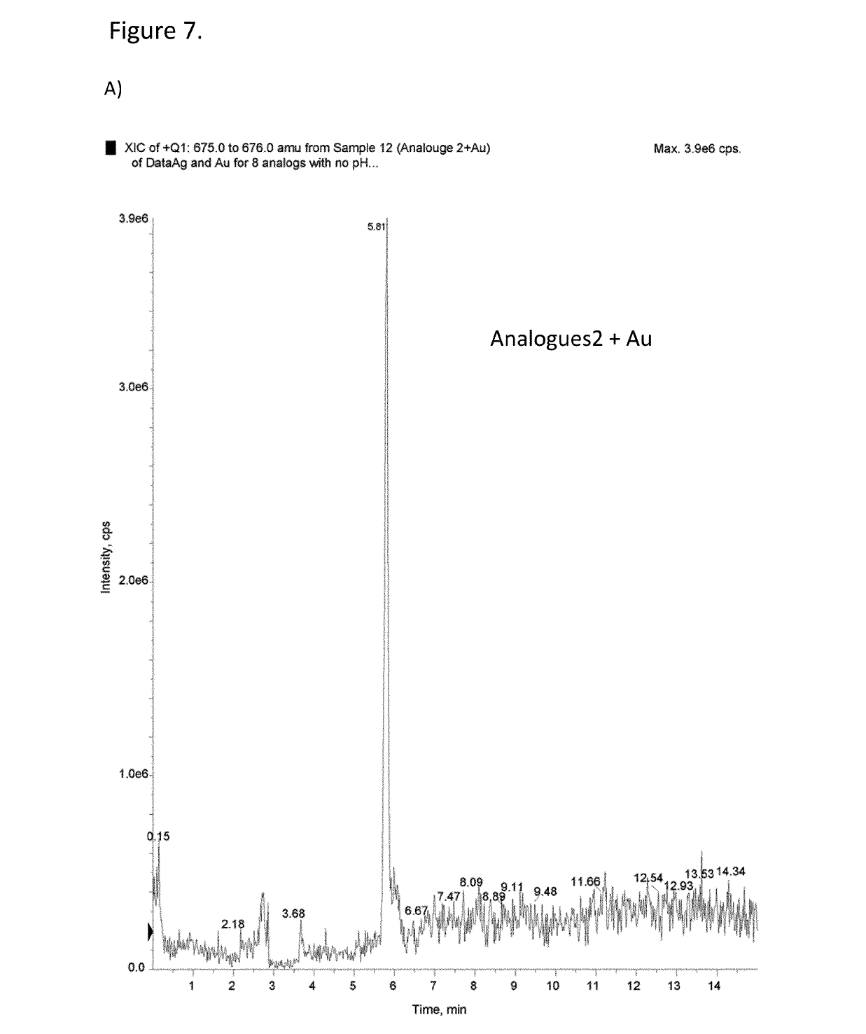

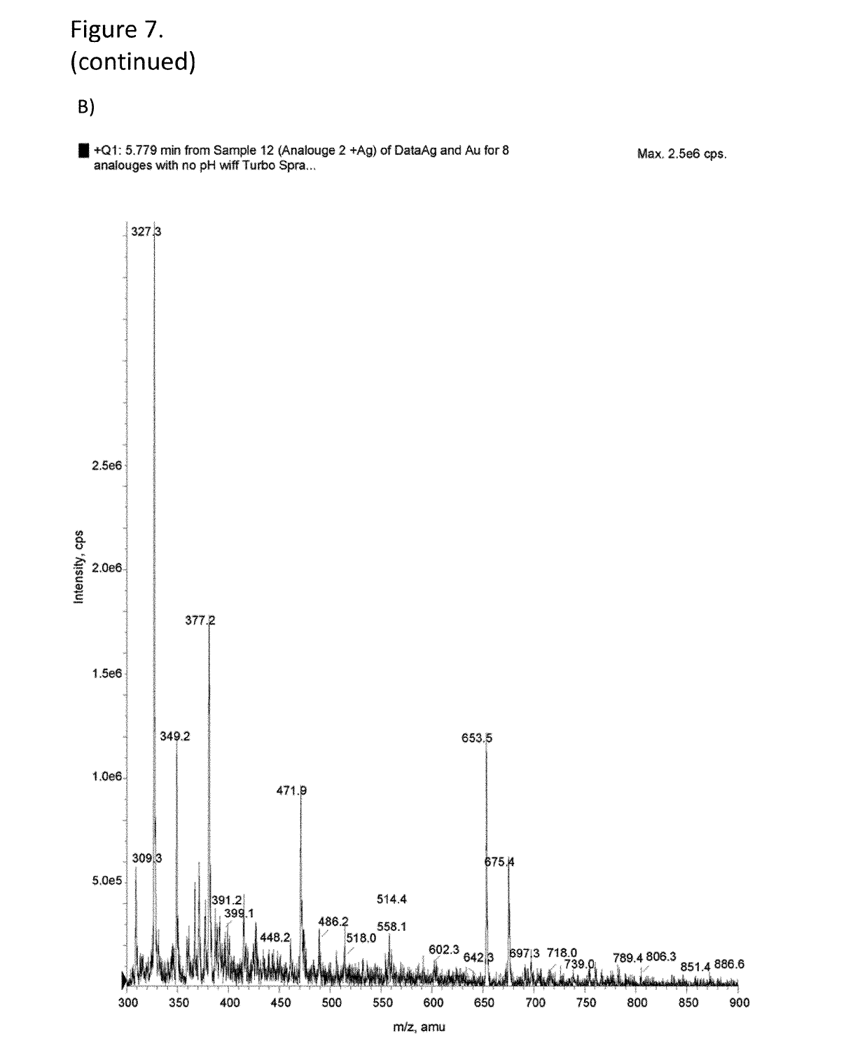

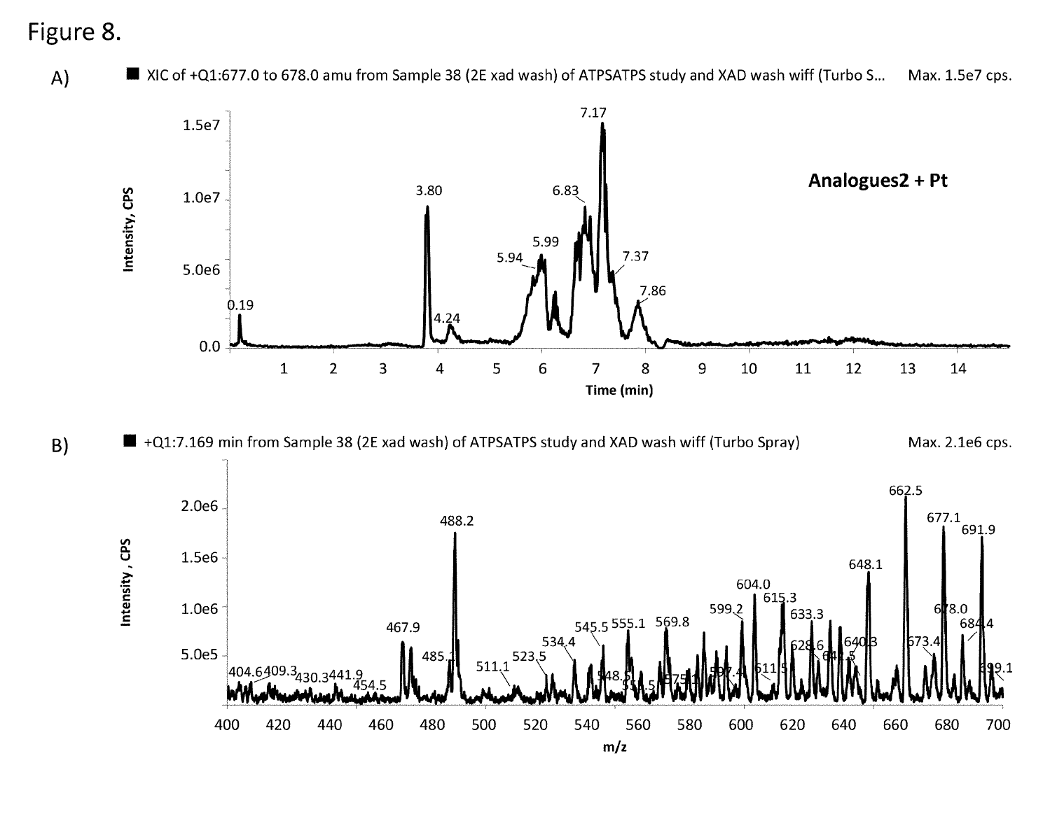

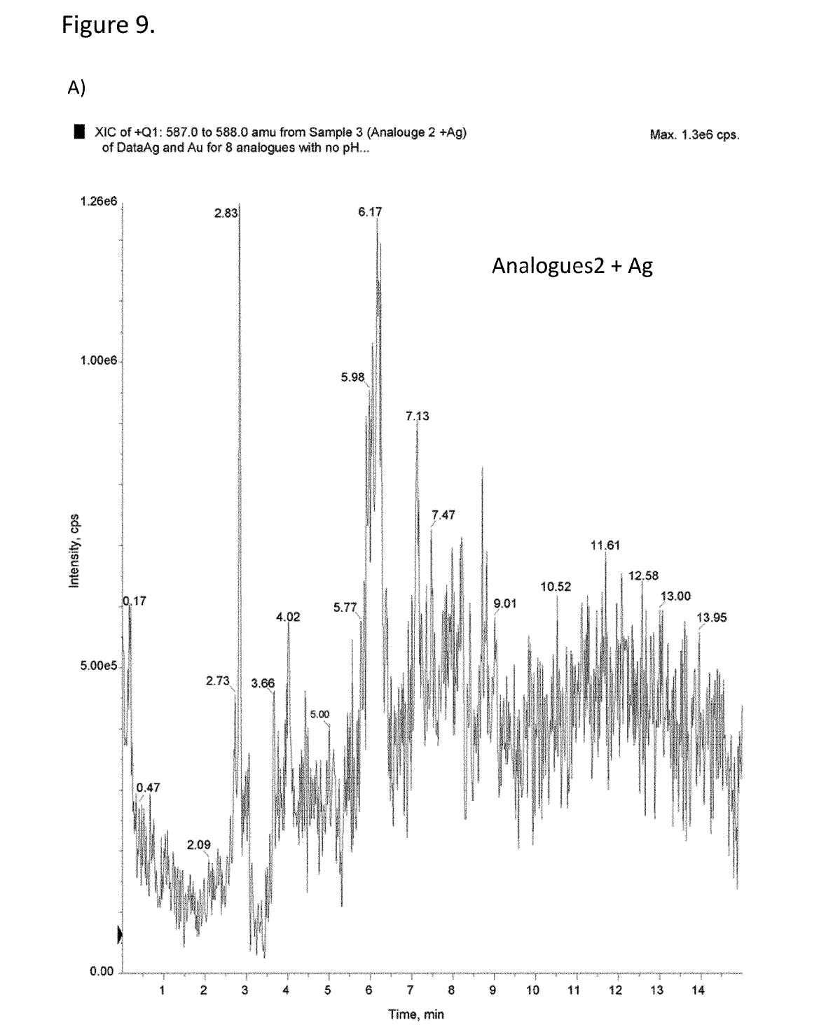

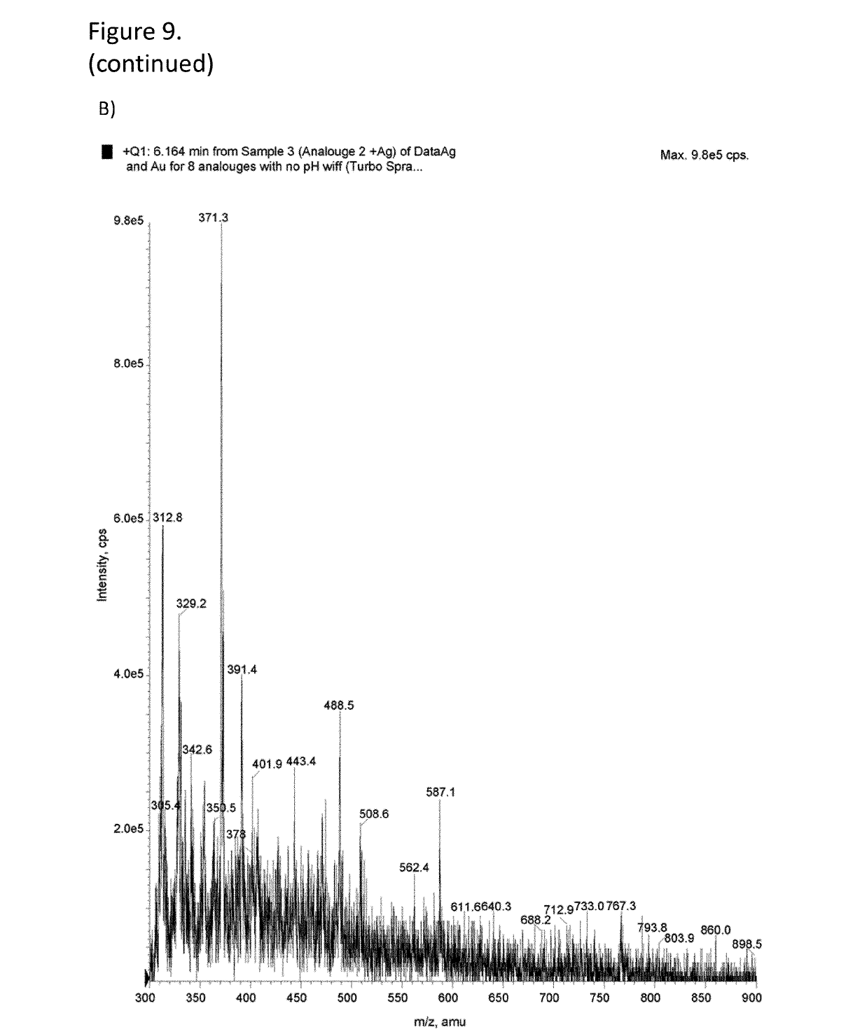

We then tested the absorbance of different metals by the analogs in FIG. 3 under various conditions. Analog 3 is Ybt. Details are provided in the description of the drawings (FIGS. 5-6). Additional data is presented in FIGS. 7-9. Analogs 2, 4 and 6 showed the best Pt absorbance in the presence of base metals, while analogs 2 and 4 showed the best Au absorbance in the presence of base metals. In the absence of base metals, the absorbance of Pt decreased, but analog 2 was the best. In the absence of base metals, analogs 2, 4 and 6 were the best for gold. Additional conclusions are evident from the figures. When Cu is bound by Ybt or its analogs, it turns color, providing a helpful indicator.

The Ybt and analog XAD beads (40 mg) can be added to small-scale (10 mL) samples of wastewater. Incubation can occur at 30° C., and after specific time points, beads will be separated (by gravity settling) and washed with Millipore water twice. The metal content of the wastewater samples before and after treatment can be measured using inductively coupled plasma mass spectrometry (ICP-MS). Alterations on wastewater treatment can include successive addition of Ybt or analog XAD beads to samples to test for staged separation capability. Treatment efforts can also be extended to larger-scale (0.1-1 L) wastewater samples for metal recovery.

Those metals bound and removed by Ybt can be used for metal recovery and re-use. To recover metal from the Ybt-metal-XAD beads, methanol can be used to remove the Ybt-metal complex. Data shows 90% of Ybt was removed from XAD beads when washed with methanol. The metal can be recovered from the Ybt via pH variation to eliminate chelation. Variation in pH can remove the recovered metal while re-generating the Ybt-XAD particle for future wastewater treatment. Once free metal has been recovered in solution, it can be recycled back to electroplating processes. If needed, purification steps (using pH-based separations) can be used to enrich certain metals for coating purposes. In this capacity, Ybt analogs that are selective for certain metals are especially beneficial.

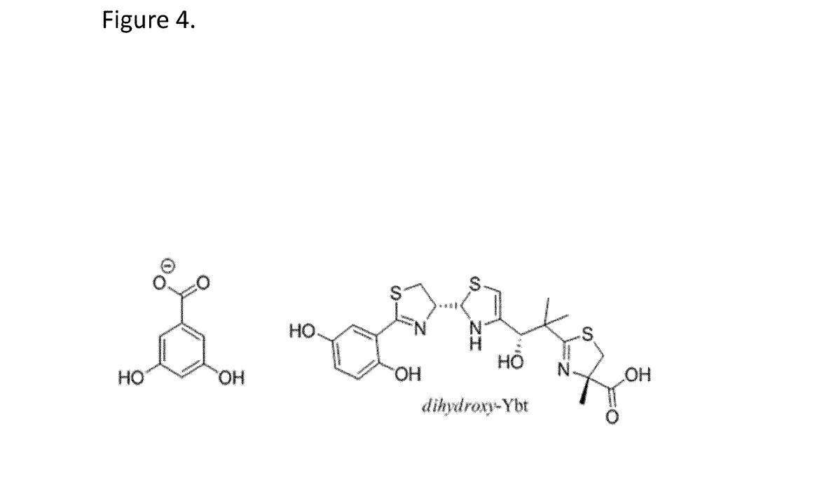

Using the same strategy outlined for the Ybt analog introduced above, additional analogs can be tested. The anthranilate-based analog presented in FIG. 2 and the analog introduced in FIG. 4 were chosen based upon the potential to selectively bind palladium, rhodium, platinum, and ruthenium common to heavy metal wastewater streams. Each starter unit can be supplied to production cultures at the time of gene expression induction, followed by extended culture (seven days) prior to extraction with ethyl acetate and analog production analysis by HPLC (described below). Production optimization steps for Ybt analogs can mirror those described above for the original Ybt compound. We anticipate significant overlap in the analog optimization steps given biosynthetic similarities to the native Ybt compound and will first apply the optimized processes determined for Ybt before altering conditions to improve analog production further.

The metabolic and process engineering efforts can be analyzed across production metrics that include product titer, specific productivity, and volumetric productivity. Production of Ybt can be analyzed by HPLC analysis when compared to a Ybt standard and using the extinction coefficient for the compound as described previously. Product titers can also be analyzed over time with respect to dry cell weight (monitored by optical density measurements made at 600 nm and converted by sample collection, drying, and weight measurement) and culture volume. The same analysis can be applied to Ybt analogs using Ybt as a standard (as was done to quantify anthra-Ybt [FIG. 2]). It is acknowledged that this will be an approximation for analog measurements, but this course of action will be pursued due to the lack of purified analogs available for quantification purposes. However, results are expected to provide a good approximation of analog production metrics.

The analysis methods discussed above can allow an assessment of the effectiveness of Ybt engineering efforts and analog formation. Goals include improvements in product titer and productivity for native Ybt as a function of engineering approaches. A key goal of analog formation is product titers that approach those of the original compound. Both goals are meant to support a future production process for applications such as the wastewater metal removal and recovery.

Similarly, process engineering strategies can be applied towards improved heterologous compound production. In particular, parameter optimization and fed-batch bioreactor approaches can be utilized to maximize volumetric productivity. More specifically, a 96-well format can be combined with a Plackett-Burman statistical screen to identify and formulate an enhanced production medium for polyketide biosynthesis. Both batch and fed-batch bioreactor schemes can be used to boost heterologous biosynthesis of complex natural products.

Variations in the conditions are considered within the scope of the present disclosure. For instance, pH, temperature and other parameters may be varied to optimize metal binding and release. XAD may be fixed and Ybt or its analogs bound and removed as needed to treat wastewater streams. In this type of operation, the cost of XAD can be minimized while the continual application and removal of Ybt compounds can leverage the process optimization. For example, XAD and Ybt or its analogs may be in a cassette or cartridge.

We can also test metal removal from wastewater samples using the native Ybt molecule. In this regard, the native compound demonstrated the ability to remove Fe³⁺ and Cu²⁺ from aqueous laboratory samples. The Ybt compound and associated analogs can be tested for additional metal removal capacity when using actual field wastewater samples. Furthermore, metal recovery for re-use in manufacturing processes can be tested. Use of Ybt can result in a small-scale wastewater metal removal, recovery, and re-use process for industries facing metal removal and recovery challenges.

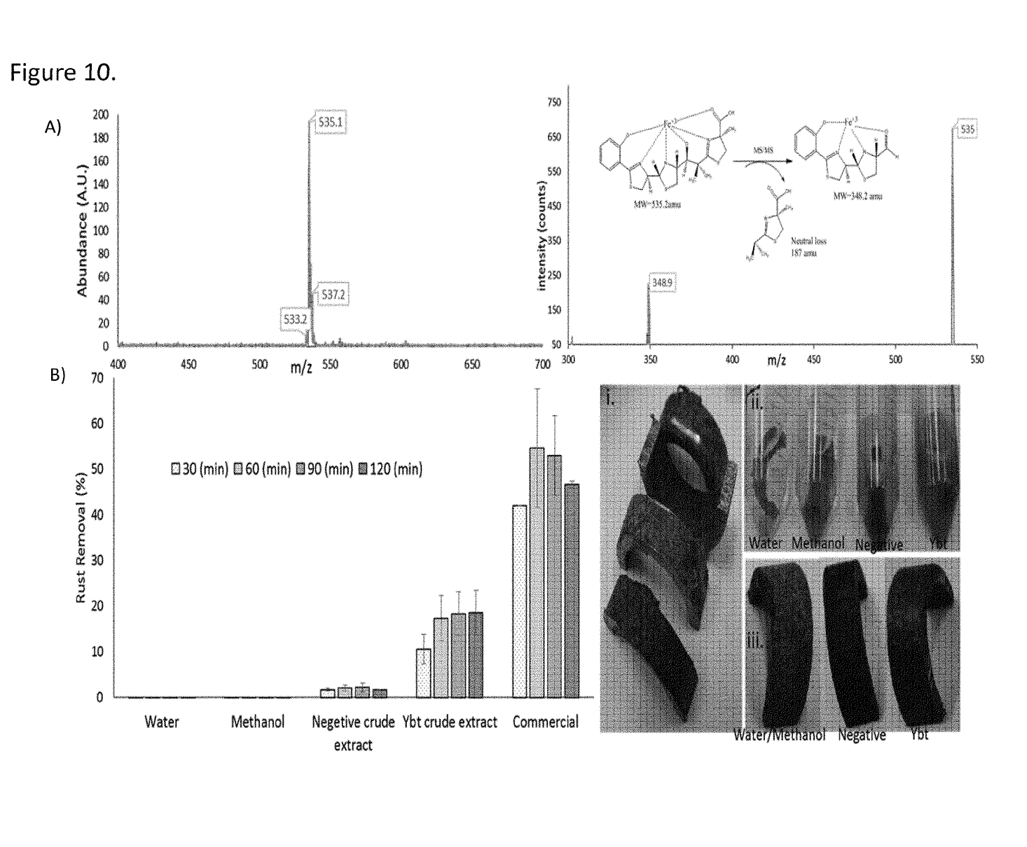

We utilized Ybt in two capacities as a metal binding product. In the first, the native capability of the compound was used to chelate iron in the context of rust removal. Crude extracts of Ybt-producing cultures were tested against a series of controls (including a commercial rust removal product) to assess the capabilities of the compound in this context. As shown in FIG. 10, the Ybt extract removed ˜20% of total rust and ˜40% relative to the commercial control.

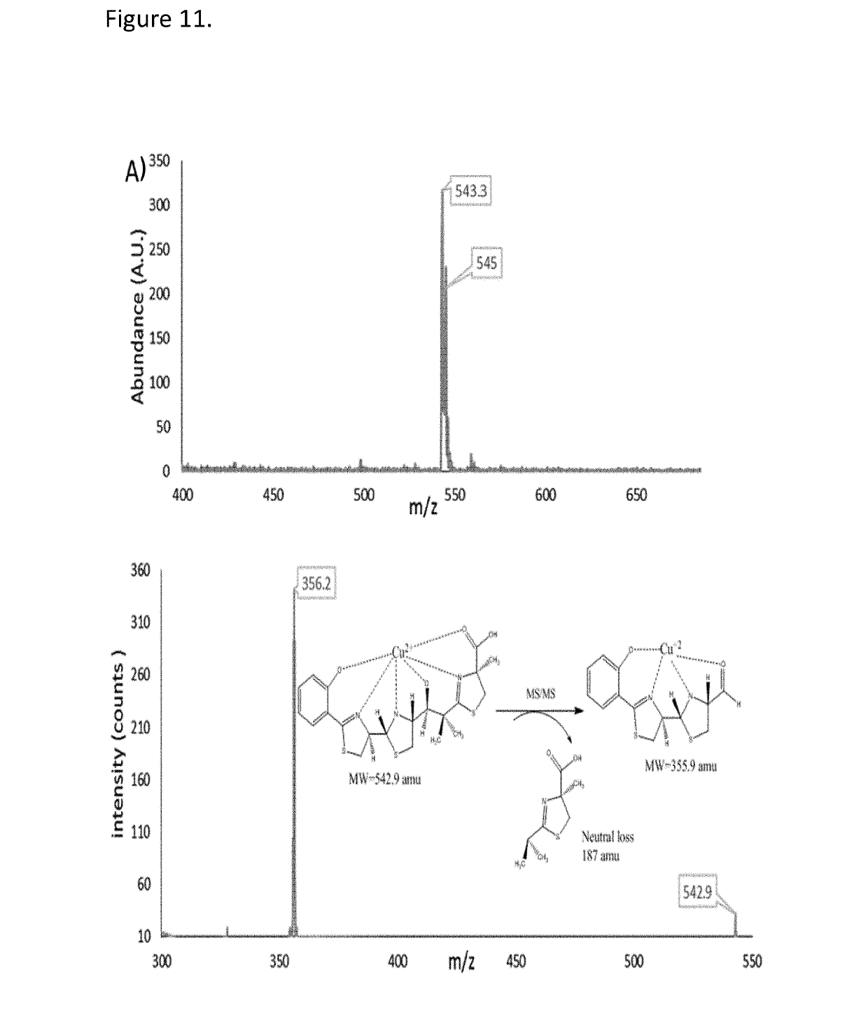

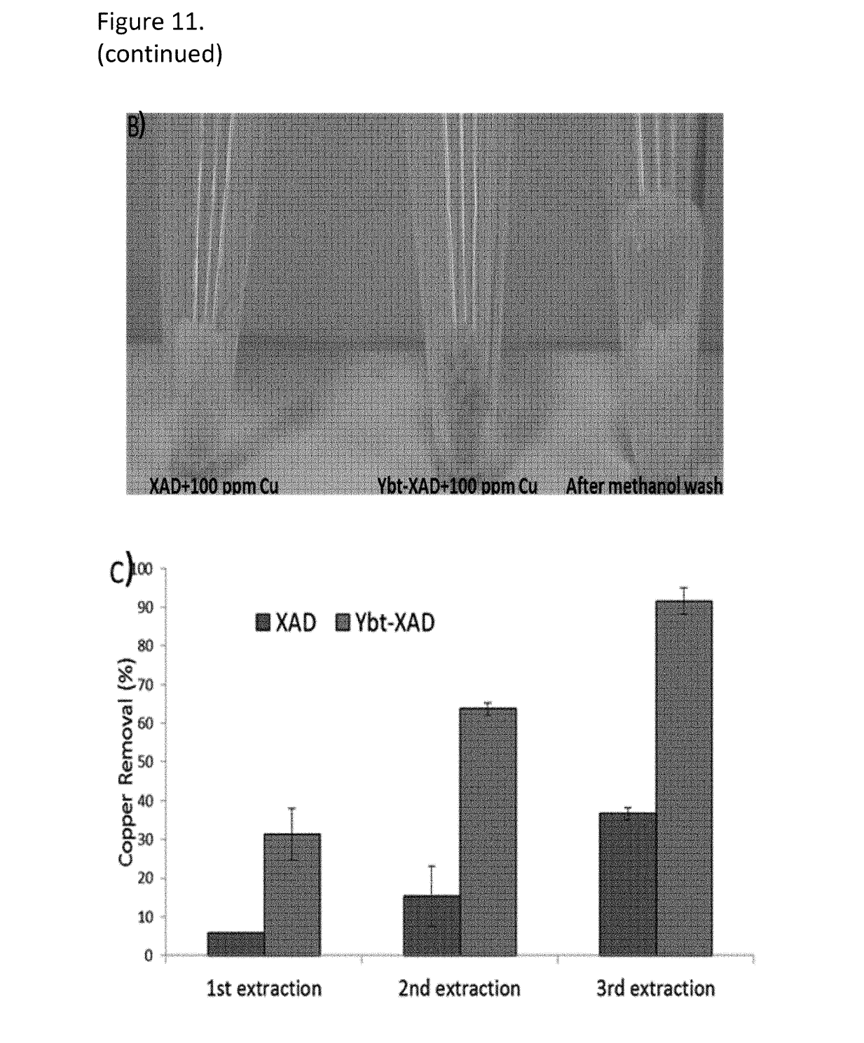

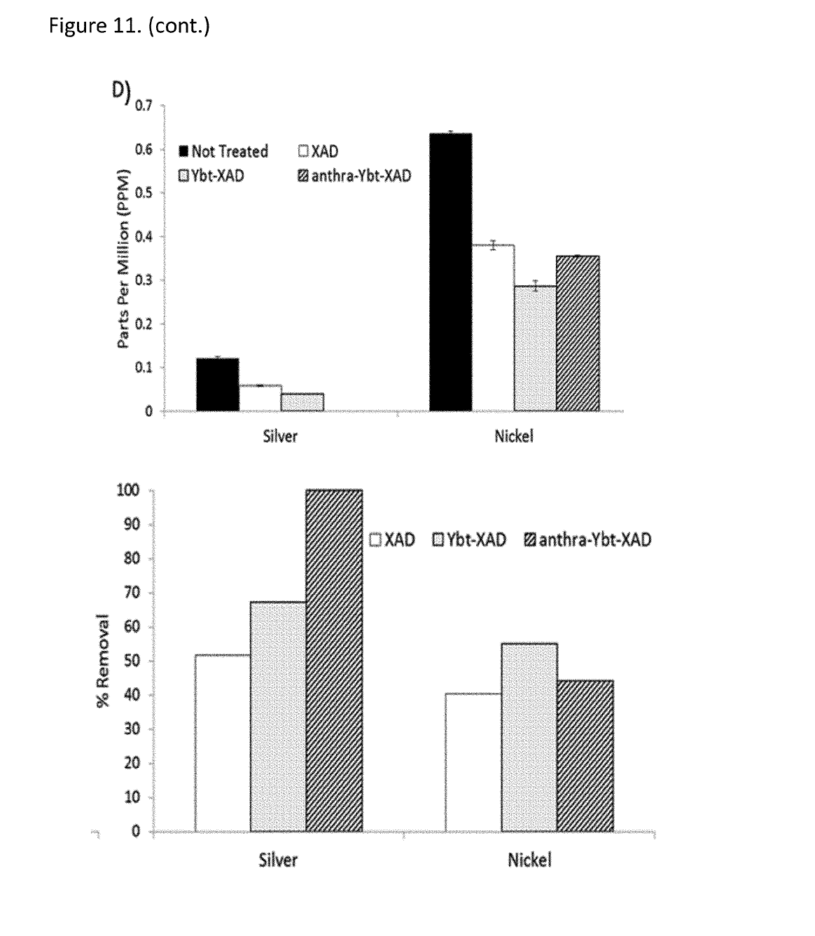

Extending the metal binding potential of Ybt further, a second target was selected in copper, which is a key contaminant of plating wastewater streams. In preparation for removal assays, Ybt was first adsorbed to XAD resin beads to enable a solid matrix readily separated from copper-containing fluids. Ybt attachment to XAD was nearly quantitative as >95% was adsorbed; conversely, when Ybt-modified XAD beads were incubated with methanol, 90% of Ybt was removed (FIG. 6B). The Ybt matrix then allowed a simple means of testing a solid-phase separation process for fluids containing copper. This was demonstrated in FIG. 11. Across panels A-C, Ybt-modified XAD demonstrated clear removal capabilities compared to controls. Repeated extractions with Ybt resulted in nearly 100% full solution removal (FIG. 11C).

The data for iron and copper removal supported the binding and removal potential of Ybt. To further support the use of the proposed technology, Ybt and anthra-Ybt were bound to XAD and tested with field wastewater provided by Precious Plate. As demonstrated in FIG. 11D, selective removal was observed for silver and nickel. Of note, the anthra-Ybt-XAD beads demonstrated 100% removal of silver during an initial assessment.

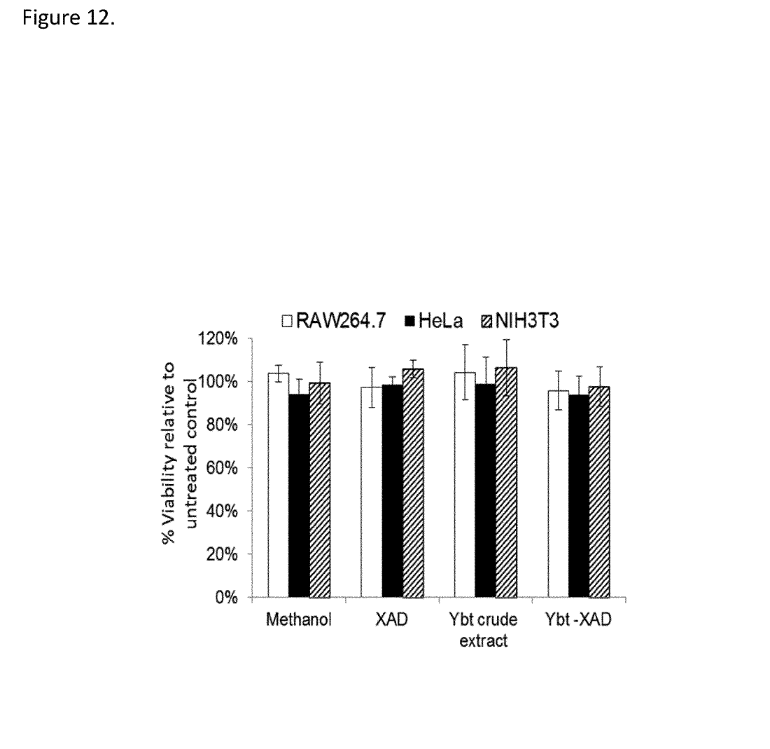

Finally, the heterologous production process for Ybt removes any need to deal with the native production host (in this case, a priority pathogen) during subsequent applications. Furthermore, the compound itself appears to pose little health risk (FIG. 12) and is expected to be benign environmentally based upon its composition of biological building blocks. Hence, the production process for Ybt and analogs is economical, effective, and safe.

In a further development, the biosynthetic process for Ybt formation was improved through the incorporation of a dedicated step to eliminate the need for exogenous salicylate provision. Upon doing so, the compound was tested in parallel applications that highlight the metal chelating nature of the compound. Ybt was assessed as a rust remover, demonstrating a capacity of about 40% compared to a commercial removal agent and about 20% relative to total removal capacity. Next, we tested Ybt in removing copper from a variety of non-biological and biological solution mixtures. Our success across a variety of media indicates potential utility in diverse scenarios that include environmental and biomedical settings.

Materials and Methods.

Strains and Plasmid Construction. Plasmid cloning was completed using DH5α; whereas, Ybt production was completed in strain BAP1. The irp9 gene was obtained from PCR amplification of Yersinia enterocolitica genomic DNA using the following primers: irp9-F: 5′-AATCATATGAAAATCAGTGAATTTCTACAC-3′ (SEQ ID NO:9) and irp9-R: 5′-AATCCTCGAGCTACTACACCATTAAATAGGG-3′ (SEQ ID NO:10). The 1.3 kb amplified gene product was cloned into pCDFDuet-1 (EMD Chemicals Inc. [Gibbstown, N.J., USA]) using restriction sites NdeI and XhoI (underlined in the primers above). Plasmids pBP198 (pET21c containing genes encoding HMWP2 and YbtU), pBP205 (pET28a containing genes encoding HMWP1 and YbtE), and pBP200 (pGZ119EH containing the gene encoding YbtT) were described previously. Plasmids were transformed into BAP1 using electroporation. Following selection with appropriate antibiotics, resulting strain-plasmid combinations were stored as 20% glycerol stocks.

Bacterial Culture and Compound Extraction. Overnight cultures from glycerol stocks were incubated at 37° C. with shaking in lysogeny broth (LB) medium and used to inoculate (1% v/v) either 25 mL (Ybt production comparisons) or 500 mL (Ybt extraction) cultures of M9 minimal medium (per liter: 12.8 g Na₂HPO₄.7H₂O; 6 g Na₂HPO₄; 3 g KH₂PO₄; 0.5 g NaCl; 1 g NH₄Cl; pH adjusted to 7.4 with NaOH) supplemented with glycerol (0.5 wt %) and casamino acids (1 wt %). Post-inoculation, cultures were incubated at 22° C. with shaking for five days with induction initiated at an OD600 nm of 0.4-0.6 using 100 μM isopropyl β-D-1-thiogalactopyranoside (IPTG). For those cultures requiring exogenous supplementation, 1 mM salicylate was added at induction. Culture plasmid selection was maintained with 100 μg/mL ampicillin, 50 μg/mL kanamycin, 20 μg/mL chloramphenicol, and 50 μg/mL spectinomycin, as needed (the same antibiotic levels were used for solid medium transformation selection). Post-culture, 1 mL acetone was added per 50 mL of medium and incubated for an additional 30 minutes prior to centrifugation to collect the resulting supernatant for extraction. For smaller-scale cultures used to compare Ybt production levels, Fe³⁺ was added to culture supernatants at a final concentration of 5 mM followed by ethyl acetate extraction twice with an equal volume of ethyl acetate each time. Supernatants collected from larger-scale 500 mL cultures were extracted twice with an equal volume of ethyl acetate each time. Extracts from respective cultures were combined, evaporated to dryness under vacuum, and the resulting residue was resuspended in methanol (with the final volume concentrated 100× relative to the original culture volume). Where indicated, negative control samples were strains without pBP205.

HPLC and LC-MS Analysis. Salicylate quantification was completed using a ZORBAX Eclipse XDB-C18 column connected to an Agilent 1120 system equipped with a diode array detector. Solvent A was 0.1% formic acid in water, solvent B was methanol, and samples (20 μL culture supernatant) were analyzed at a flow rate of 1 mL/minute using the following gradient: 5-50% solvent B for 15 minutes, 50-5% solvent B for 1 minute, and 5% solvent B for 4 minutes. An absorbance wavelength of 304 nm was used and peak area quantification was conducted compared to a standard calibration curve of pure salicylate (Sigma-Aldrich).

For initial Ybt detection, HPLC was completed using the same system described for salicylate analysis. Injected final extract samples (20 μL) were subjected to a gradient of acetonitrile (0-2% from 5-7 minutes; 2-70% from 7-20 minutes; 70-80% from 20-22 minutes; and 80-0% from 22 to 27 minutes) with an initial base buffer of 17 mM formic acid (pH 3.35) using a flow rate of 1 mL/minute. The Ybt-Fe³⁺ product was detected at 385 nm. Purified product was achieved using an Agilent 1200 preparatory HPLC system equipped with a Waters C18, 5 μM, 300 Å, 150×3.9 mm ID column. In this setting, a 1 mL/minute flow rate was used together with a 10-100% acetonitrile (balance water) gradient over 15 minutes. Collected fractions containing product were quantified at 385 nm using the known extinction coefficient for Ybt-Fe³⁺ (ε=2,884). Purified product was then used to produce a calibration curve for compound quantification via LC-MS analysis using an API 3000 Triple Quad LC-MS with a Turbo Ion Spray source (PE Sciex) coupled with a Shimadzu Prominence LC system. All MS analyses were conducted in positive ion mode, and chromatography was performed on a Waters X Terra C18 column (5 μm, 2.1 mm×250 mm). After an injection of 4 μL of final extract, conditions for LC-MS were 10-100% acetonitrile (balance water) gradient over 15 minutes at a flow rate of 0.2 mL/minute.

Rust Removal Experiments. A rusted carbon steel tube was machine cut into U-shaped pieces (through the University at Buffalo School of Engineering and Applied Sciences Engineering Machine Shop) and each piece, after cleaning with acetone and rinsing with DI water, immersed in 10 mL Millipore water. Samples were then mixed with 250 μL of methanol or final extracts (about 0.2 mg of Ybt) derived from the larger-scale negative control or Ybt production cultures. Samples were then allowed to incubate at 30° C. with shaking for 2 hours.

Rust removal was quantified through a modification of the 1,10-phenanthroline assay to enable detection using a 96-well plate format. To each well, 40 μL of 0.3% (wt/vol) 1,10-phenanthroline, 40 μL of 1% (wt/vol) hydroxylamine hydrochloride, and 40 μL of 0.1 M ammonium acetate were mixed. Ten microliters of diluted samples (to obtain Fe³⁺ in a range of 1 to 10 ppm) was added to the mixture, and the total volume adjusted to 250 μL using Millipore water. Absorbance was measured using a microplate reader at a wavelength of 510 nm with concentration measurements determined from a calibration curve of Fe³⁺ (Sigma-Aldrich).

To assess total rust removal, metal samples recovered after incubation were washed with water and immersed in a solution of 1% (wt/vol) oxalic acid for 30 minute to remove the remaining rust on the metal surface. Upon quantifying the amount of Fe³⁺ removed using the 96-well plate method introduced above, percent rust removal was calculated using the following equation:

$$Rust Removal ( \% ) = \frac{R 1}{R 1 + R 2} \times 100$$

where R1 is mg of Fe³⁺ in solution after treatment with Ybt extracts or controls and R2 is mg Fe³⁺ removed with 1% oxalic acid solution.

Ybt-XAD Preparation and Characterization. Amberlite® XAD-16N beads were activated as recommended by the manufacturer (Sigma-Aldrich). Beads were washed twice with methanol and twice with water before finally being resuspended in Millipore water. Next, 250 μL of Ybt-containing or negative control final extract was added to 0.5 g of XAD beads and the final volume was adjusted to 10 mL using Millipore water. Mixtures were incubated at 30° C. overnight. Beads were then washed with Millipore water three times and resuspended in Millipore water for further tests. To assess the effectiveness of Ybt binding to XAD beads, Ybt levels were analyzed by LC-MS before and after contact with XAD.

Copper Removal Experiments. Copper and zinc removal assessment was completed using the Zincon assay (Sabel et al. 2010. Anal Biochem 397:218-226). Briefly, 40 μL samples (diluted if necessary to keep concentrations less than 4 ppm) were added to 200 μL of borate buffer (65.8 mM; pH=9), and the assay was initiated by adding 10 μL of Zincon solution (1.6 mM). After a five-minute incubation at 20° C., absorbance at 610 nm was measured. Standard curves for Zn and Cu were made using pure salts (Sigma-Aldrich) to allow quantification. The initial measurement provides total metal concentration (zinc and copper together) while the second measurement allows determination of the copper concentration. By subtracting the second reading from the first, zinc concentration is determined. Standard curves for Zn and Cu were made using pure salts to allow quantification.

Adsorption Kinetic and Equilibrium Measurements. In all experiments, Ybt-XAD (0.2 g) was added to 20 mL of solution (L/S=100). Samples (100 μL each) were measured over time (from 1 to 180 minutes) across three different initial concentrations of copper (2.5, 5, and 10 ppm). Equilibrium measurements were made by changing copper concentrations (0, 1, 2.5, 5, 7.5, and 10 ppm). Mixtures were shaked vigorously (400 rpm) for 60 minutes and then analyzed.

To test copper removal, 800 μL of copper solution was added to 40 mg of Ybt-modified XAD beads (or controls) and incubated at 30° C. with shaking (for sheep blood and 50% FBS RPMI samples, this temperature was 22° C.). After specific time points, beads were separated by gravity settling and washed with 800 μL of Millipore water twice. The copper content of the original mixture solution and the liquid resulting from the bead washing steps was measured using the Zincon assay described above. For samples contacted with YBT-modified XAD beads multiple times, freshly prepared beads were used in three successive applications with the resulting solution copper concentration measured at the completion of each stage. The removal ability was calculated using the following equation:

$$Copper Removal ( \% ) = \frac{( {C}_{i} - {C}_{f} )}{{C}_{i}} \times 100$$

where Ci is copper concentration of the initial solution and Cf is the final concentration of copper including washes. For samples containing blood or FBS components, an increased copper range of 7.5 to 20 ppm was used (above the 5 ppm level used for non-biological fluid assays) to account for non-specific binding of copper to protein and other components of these samples.

Biological Interaction Assays. Cytotoxicity resulting from Ybt samples was determined by the 3-(4, 5-dimethylthiazol-2-yl)-diphenyltetrazolium bromide (MTT) colorimetric assay. Three distinct cell lines (RAW264.7 [macrophage; provided by Dr. Terry Connell, Department of Microbiology and Immunology, University at Buffalo], HeLa [epithelial; provided by Dr. Stelios Andreadis, Department of Chemical and Biological Engineering, University of Buffalo], and NIH3T3 [fibroblast; ATCC]) were cultured in T75 flasks at 37° C./5% CO₂. RAW264.7 cells were maintained in medium prepared as follows: 50 mL of FBS (heat inactivated), 5 mL of 100 mM MEM sodium pyruvate, 5 mL of 1 M HEPES buffer, 5 mL of penicillin/streptomycin solution, and 1.25 g of D-(+)-glucose added to 500 mL RPMI-1640 and filter sterilized. HeLa and NIH3T3 cells were maintained in Dulbecco's Modified Eagle Medium (DMEM; Gibco BRL, Grand Island, N.Y.) supplemented with 10% (v/v) FBS (GIBCO) and 1% (v/v) Antibiotic-Antimycotic (A/A; Gibco). Cells were harvested using mechanical scrapers for RAW264.7 and 0.25% trypsin/1 mM EDTA treatment, trypsin inactivation using an equal volume of DMEM supplemented with 10% FBS, centrifugation (250×g, 5 min), and resuspension in phosphate buffered saline (PBS) for HeLa and NIH3T3 cell lines prior to seeding at 3×104 (RAW264.7), 2.9×104 (HeLa), and 1.4×104 (NIH3T3) cells/well in tissue culture-treated, sterile, polystyrene 96-well plates in 100 μL medium/well. Post-seeding, cells were allowed 24 h for attachment at 37° C./5% CO₂. Final extract was added to 0.03 mg/mL Ybt per well; whereas, Ybt-complexed XAD beads (or control bead samples) were added at 50 mg/mL per well (with the solid phase concentration chosen to equate to the corresponding amount of free Ybt). Following a 24 h incubation after sample addition, cells were assayed with MTT solution (5 mg/mL), added at 10% v/v for 3 h at 37° C./5% CO₂. Medium plus MTT solution was then aspirated and replaced by DMSO to dissolve the formazan reaction products. Following agitated incubation for 1 h, the formazan solution was analyzed by microplate reader at 570 nm with 630 nm serving as the reference wavelength. Results are presented as a percentage of untreated cells (100% viability). Nitric oxide (NO) production from RAW264.7 cells (a measure of immunogenicity triggered by Ybt sample addition) was determined using a Griess reagent kit (Promega, Madison, Wis.) according to the manufacturer's instructions.

To assess potential disruption of red blood cell (RBC) membranes by Ybt samples, a hemolysis assay was modified from a protocol previously described (Jones et al. 2014. Proc Natl Acad Sci USA 111:12360-12365). Briefly, a 5% RBC solution was prepared by washing sheep blood (HemoStat Laboratories) with PBS until the supernatant became clear. Next, 100 μL of purified 5% RBC solution was incubated with 900 μL of 0.03 mg/mL crude extract Ybt samples (or controls) or 50 mg/mL Ybt-complexed XAD beads (or controls) in PBS over time at 37° C. Triton-X 100 (1% solution) was used to construct a blood lysis standard curve by altering the amount of blood added to each respective sample. For example, for 50 and 100% lysis, 50 and 100 μL of purified 5% RBC solution was mixed with 1% Triton-X (to 1 mL). PBS was used to generate negative controls. Samples were centrifuged and hemolysis quantified by measuring supernatant at 541 nm and comparing to the % blood lysis standard curve.

Statistical Evaluation. Unless otherwise indicated, data presented were generated from three independent experiments. Error bars represent standard deviation values. All statistical significance comparisons between groups were performed using a one-way ANOVA with Dunnett (to compare within groups) post-tests.

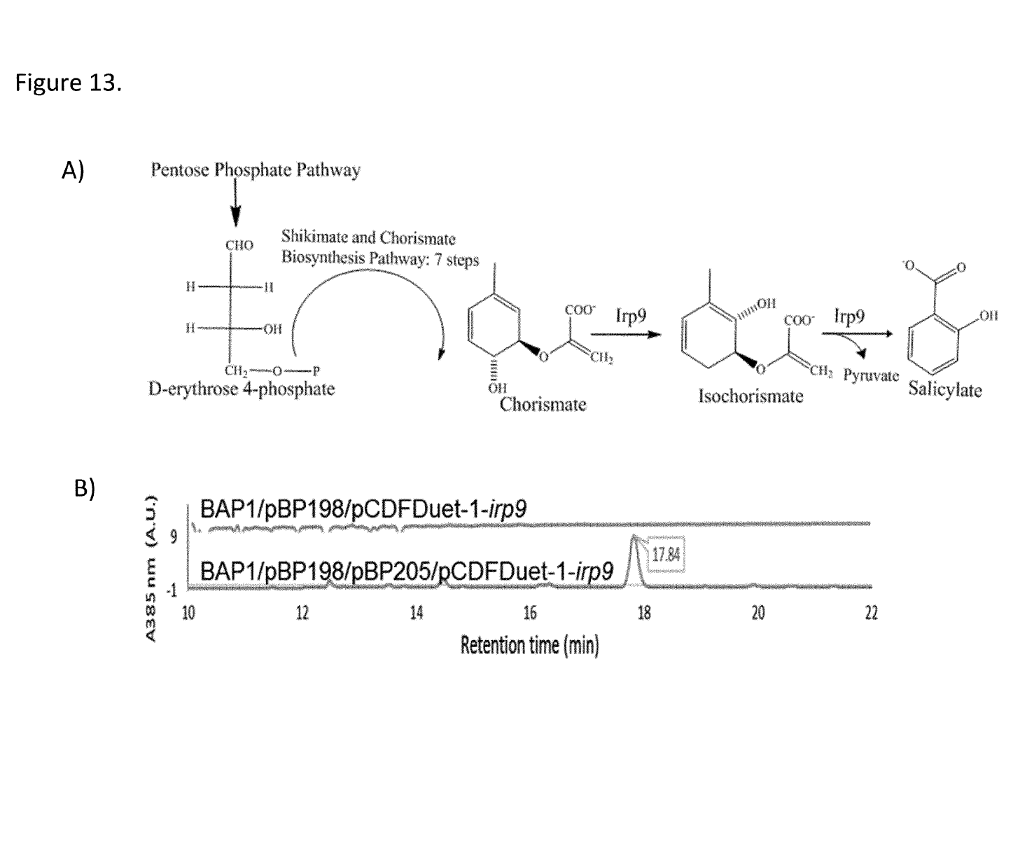

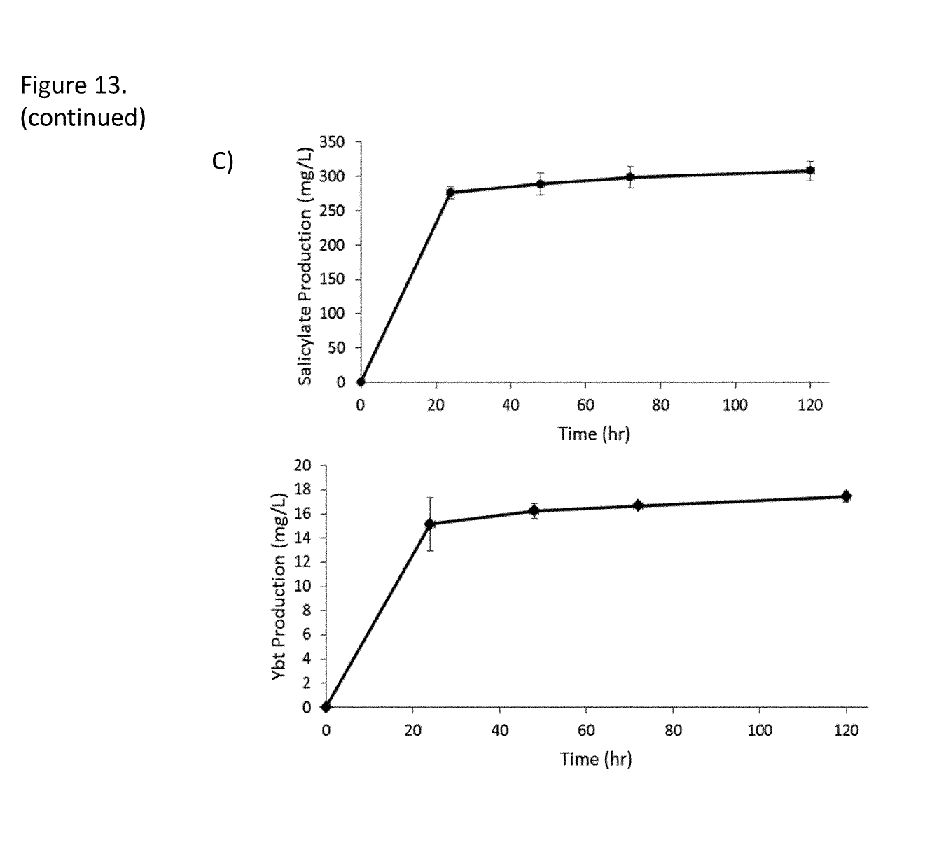

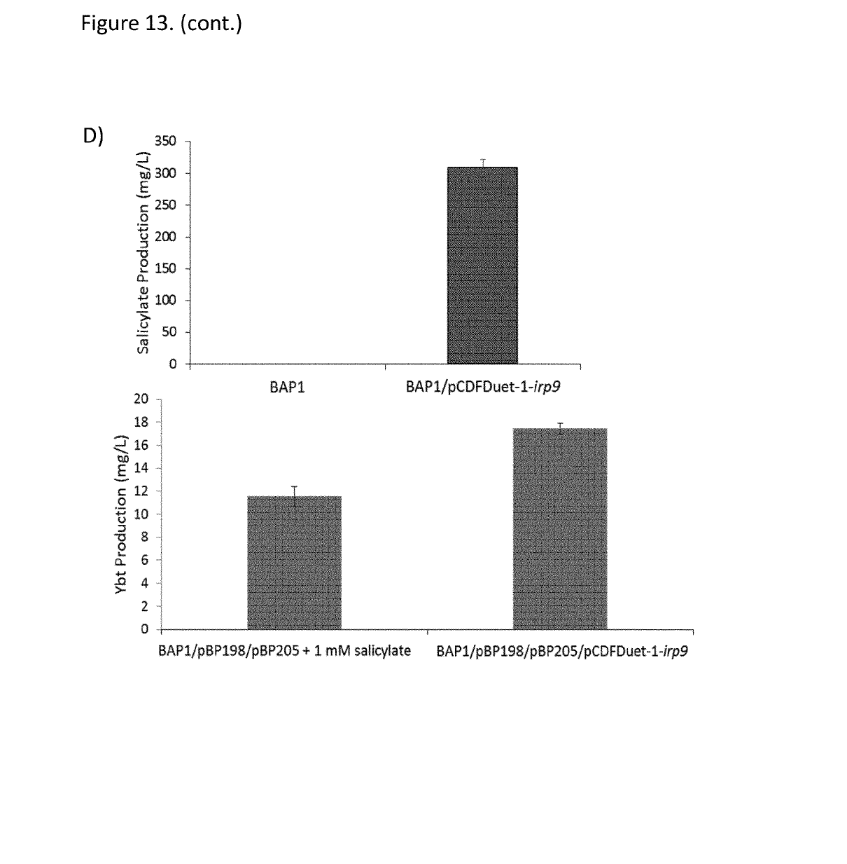

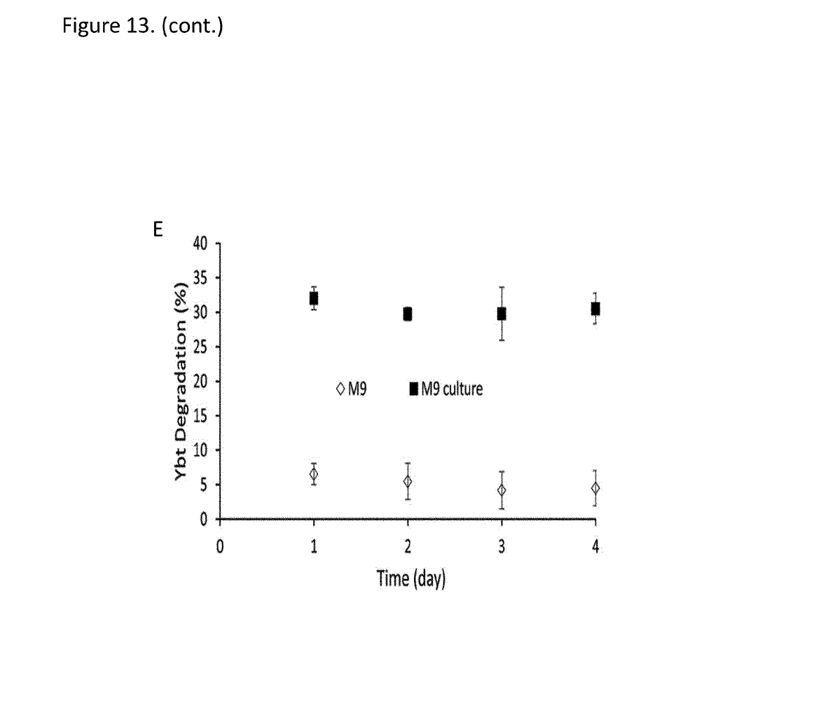

In these experiments, the irp9 gene from Y. enterocolitica was introduced to E. coli (via plasmid) to enable the intracellular provision of this starting substrate (FIG. 13A). The irp9 gene was isolated from a close relative of Y. pestis (Y. enterocolitica) and is dedicated to generating salicylate in the context of Y. enterocolitica Ybt formation. As such, this gene was transferred to E. coli as a route to intracellular salicylate formation towards heterologous Ybt biosynthesis. Successful transfer was first confirmed by the accumulation of salicylate in cultures of BAP1/pCDFDuet-1-irp9 (FIG. 13C). The irp9 gene was then tested in the context of Ybt production in comparison to previous production systems which required salicylate medium supplementation (FIG. 13D). Improvement in production resulted when using the strain capable of allowing complete intracellular production of Ybt.

The intracellular availability of salicylate removes the need for supplementation and consolidates the overall biosynthetic scheme. The introduction of irp9 also ensures that the biosynthesis can be derived from standard carbon sources (such as glucose or glycerol). The titers achieved for heterologous salicylate production were substantial, reaching ˜310 mg/L, which surpassed concentrations used previously (138 mg/L) during experiments where salicylate was added exogenously. Thus, the boost in Ybt production when using the irp9 pathway suggests a potential bottleneck in exogenous salicylate levels used during previous biosynthetic attempts.

The iron binding capabilities of Ybt offer the possibility of widespread applications related to selective metal removal. One such outlet for this activity is in the removal of rust. Rust development proceeds in a layered arrangement and features different types of iron oxide deposits which are visually distinguishable based upon color differences. Brownish surface layers possess the highest oxygen content, usually FeO(OH) and Fe(OH)₃, while the deepest layers consists of black magnetic oxide (Fe₂O₃). The desire to prevent rust development or remove rust once formed prompted us to test Ybt in a removal setting. Extracts of Ybt-producing cultures were tested against a series of controls (including a commercial product [Rust-Oleum]) to assess the rust removal capabilities of the compound (FIG. 10). As shown, the Ybt extract removed ˜20% of total rust and ˜40% relative to the commercial control. It is anticipated that further optimization parameters (Ybt concentrations, temperature, repeated applications) will further enable improved rust removal capabilities.

However, the compound may only target and remove certain components of a rusted product (such as oxides containing of Fe³⁺) or immediately proximal rust layers, and this likely contributes to the inability of the compound to fully match control samples in total rust removal. In our experiments, brownish surface layers could be removed using Ybt but the deeper black oxide layers remained (FIG. 10B).

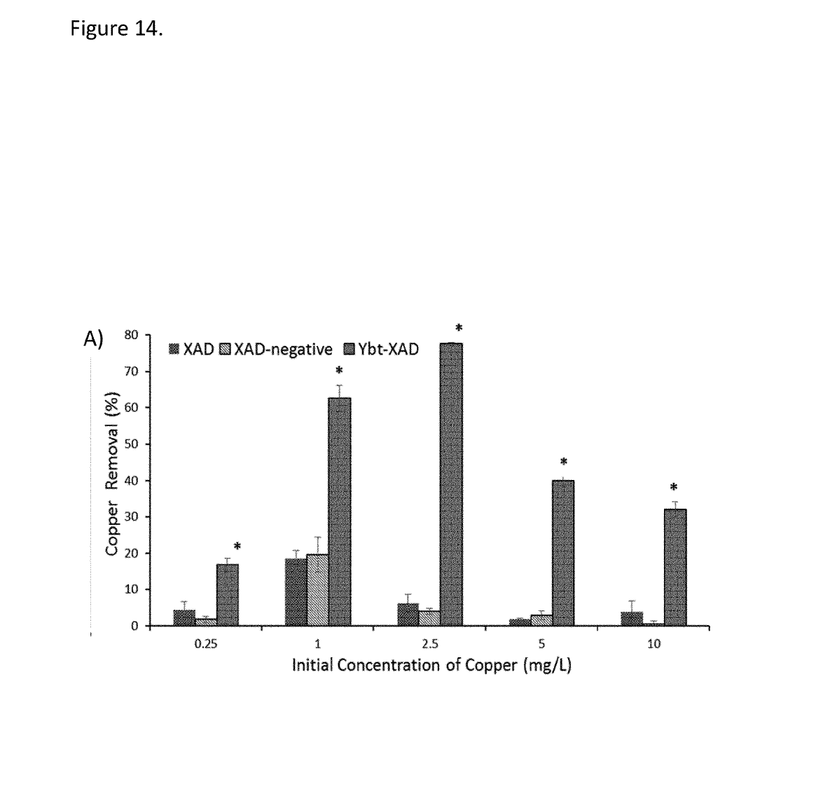

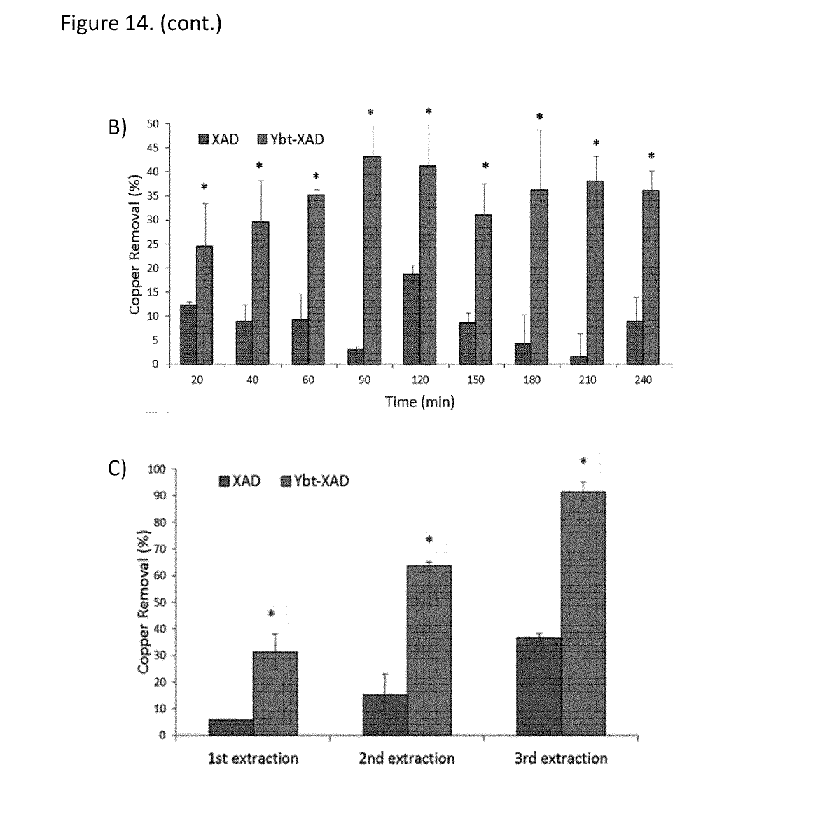

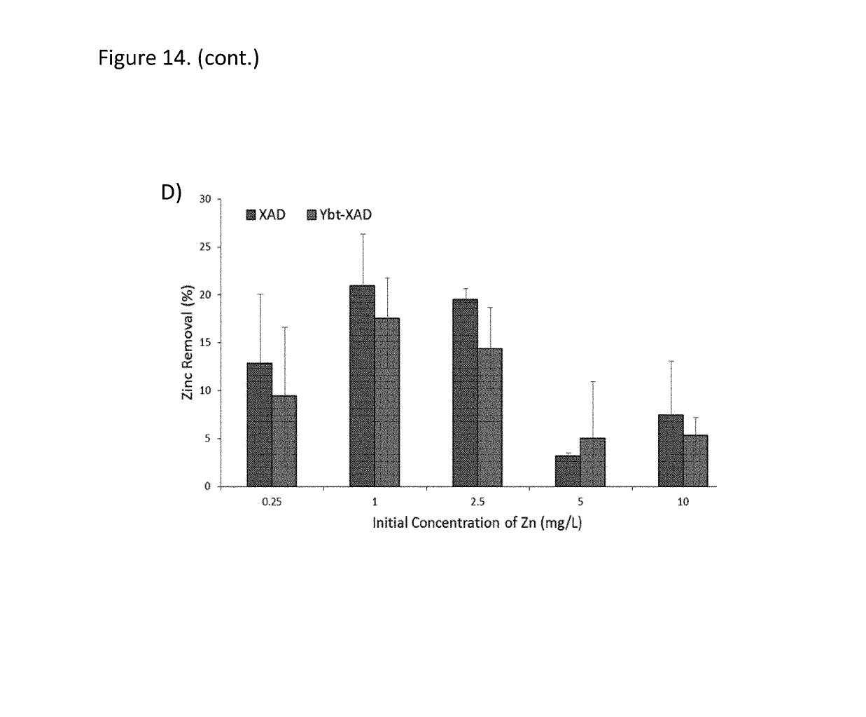

Increasing concentrations of initial copper and time course analysis resulted in a saturation in removal capability (FIGS. 14 A&B). However, repeated extractions with Ybt resulted in nearly 100% full solution removal (FIG. 14C). Importantly, Ybt showed no discernible capability to remove zinc (FIG. 14D). Regarding this last point, copper and zinc are often found together in environmental and biomedical settings. In a biomedical context, the potential of Ybt to selectively remove copper would address limitations of current treatments which bind both metals.

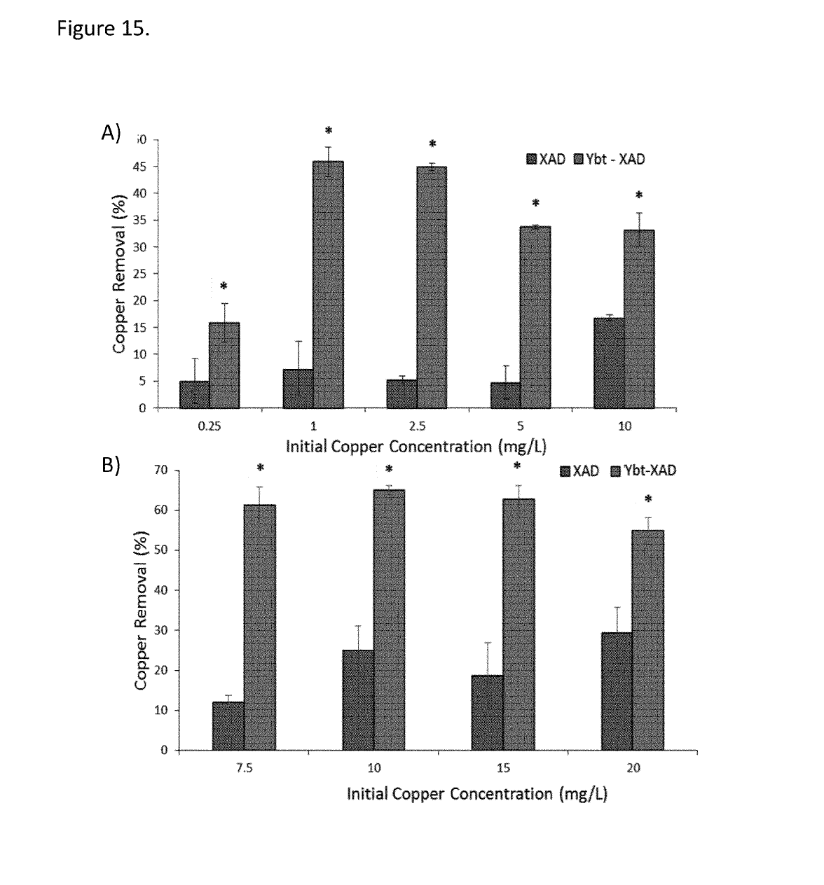

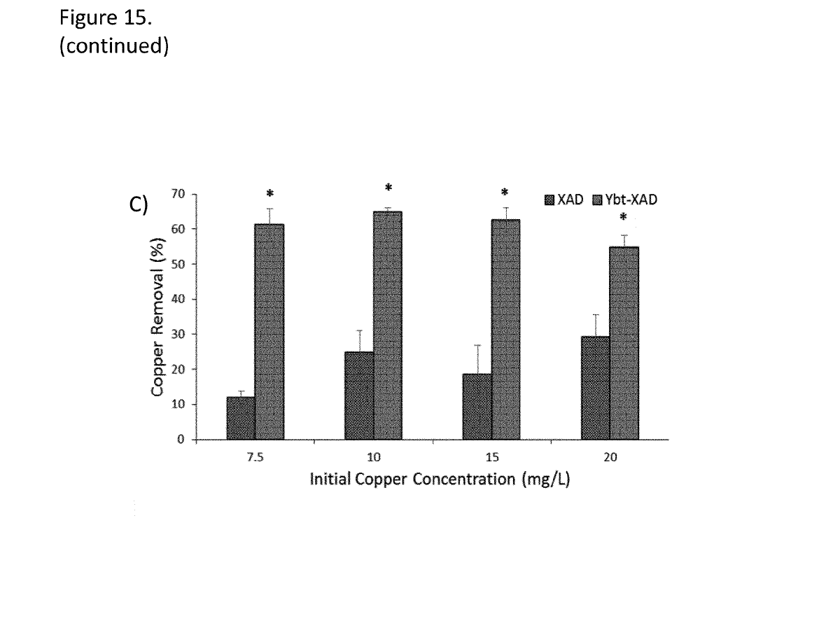

Copper removal experiments were further conducted in media representative of biological settings, including RPMI mammalian cell growth medium (with and without serum addition) and a solution of sheep blood (FIG. 15). As such, the solutions utilized include biological components, including serum proteins and red blood cells, expected to limit or inhibit the binding properties of the Ybt samples. Background binding of the XAD control was observed which is possibly explained by the non-specific adhesion of proteins and other medium components that have bound copper. In each case, however, the copper removal capability of the Ybt-modified XAD beads was maintained at levels significantly improved relative to controls. The data provide further support for copper removal in biomedical applications. In such situations, there is also the prospect of using free Ybt in an in vivo setting as opposed to the likely use of Ybt-XAD beads in an ex vivo format.

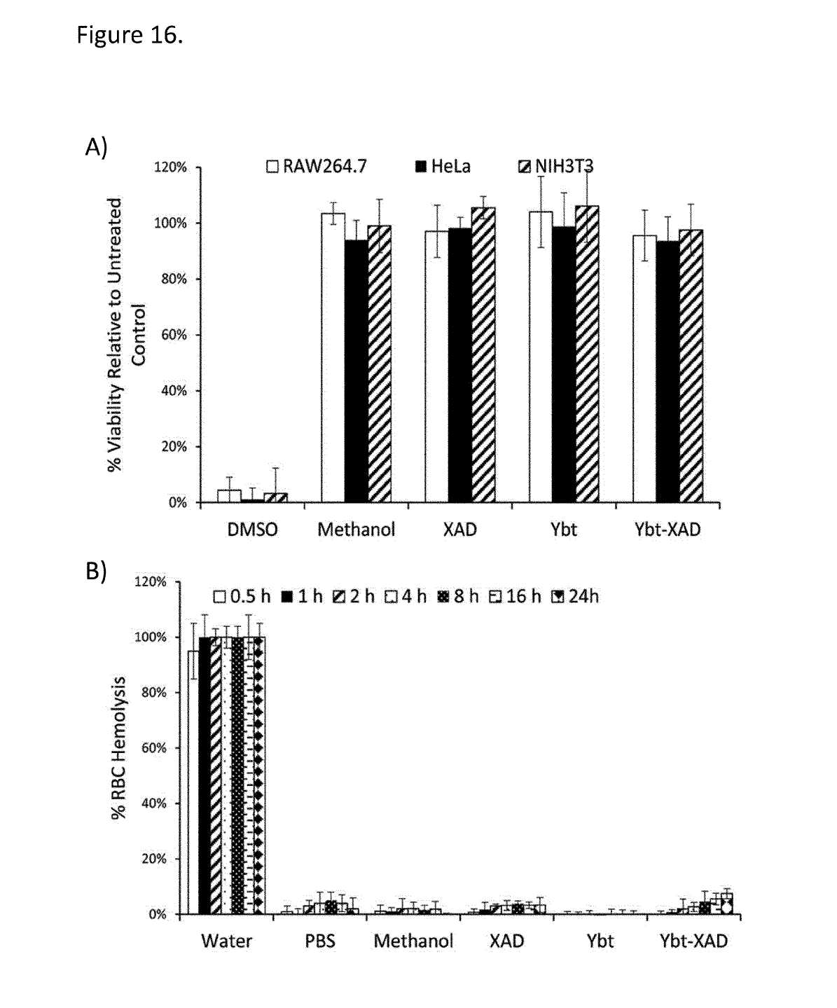

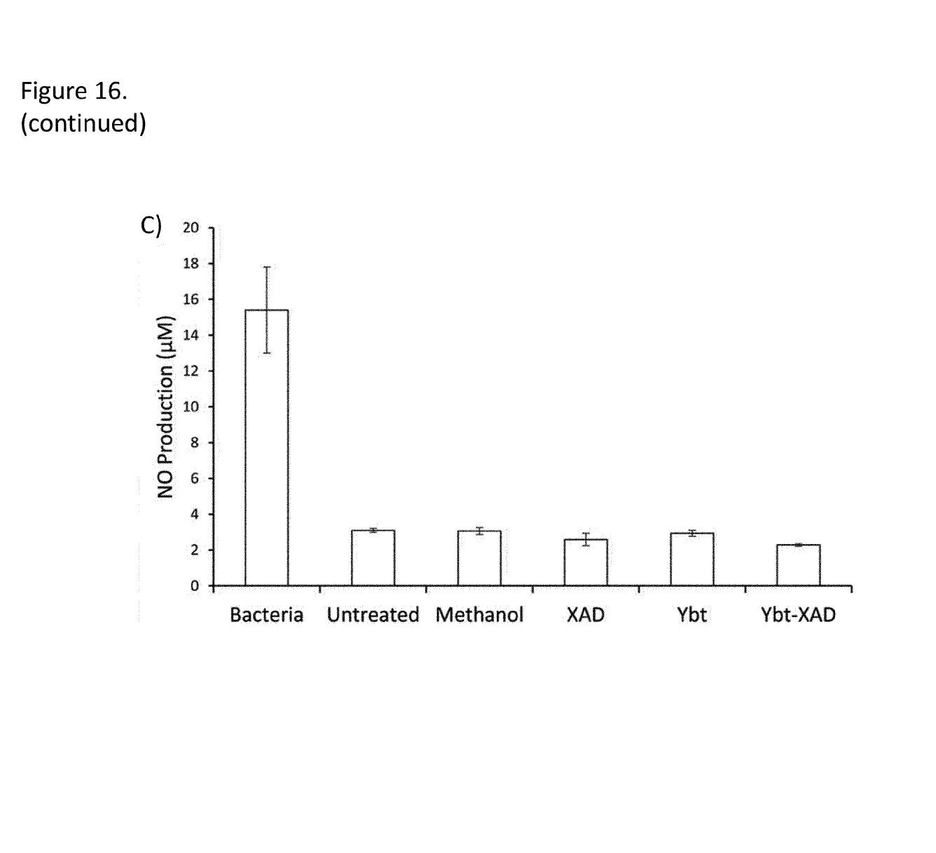

A final test of biological compatibility was conducted as presented in FIG. 16. Here, the Ybt samples were tested for detrimental biological effects. Namely, samples were assessed for 1) negative impact upon mammalian cellular viability (using three distinct cell lines); 2) unwanted immunogenicity (via NO production from macrophage cells); and 3) RBC hemolysis (a significant concern given the iron-binding properties of Ybt). In each case, the Ybt samples exhibited minimal concerns, further supporting the potential use for metal scavenging applications within biological and other settings and the likely benign environmental impact of the compound.

### Example 3

This example describes methods of using Ybt and Ybt analogs.

The performance of a commercially available scavenger (QuadraPure TU, a product of Johnson Matthey Scavenging Technologies) was compared with the instant resin-based chemistry. Due to limited access to measuring instrument(s) (ICP-MS), we used a copper solution as a wastewater effluent model. Despite this drawback, precious metal removal of 50 ppb concentrations were conducted for the developed resin. In comparison, the commercial resin must be incinerated after loading the metals to recover the bounded metal, while our new developed resin can be easily regenerated by a simple backwash process.

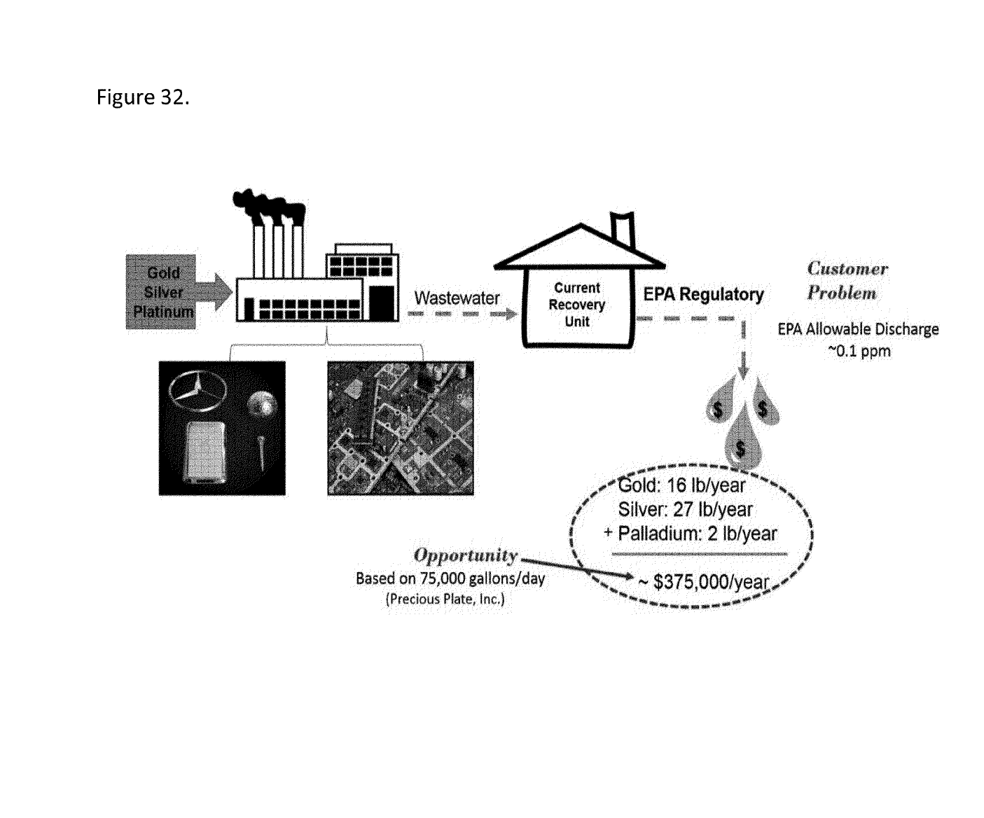

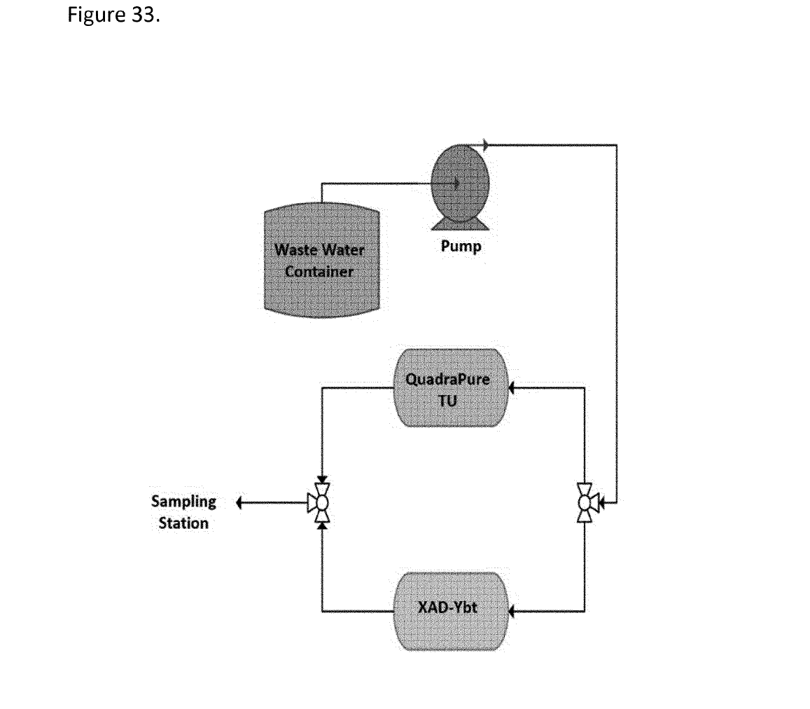

Materials and methods. Our success in heterologous production of Ybt established a production platform independent of handling the native Y. pestis pathogen and benefiting from the well-studied recombinant features of E. coli. We also observed it might be possible to engineer the molecular structure of Ybt through the application of precursor-directed biosynthesis. As demonstrated in FIG. 32, the Ybt pathway has shown flexibility in accepting alternative starter units. Produced Ybt has been utilized in capacity as a metal binding product. Copper was selected as the binding target. In preparation for a practical removal assay, the produced small molecule, Ybt, should be immobilized first to ease downstream processes. Therefore, we screened commercially available micro beads for their ability to fix Ybt on the surface. We realized polystyrene resins (specifically, XAD16) showed the most effective adsorption capability, up to 95% of Ybt was adsorbed in 2 hours. In addition, washing with methanol can elute almost all of the Ybt from the beads. The Ybt matrix then allowed a simple means of testing a solid-phase separation process for fluids containing copper. This is demonstrated in FIG. 33. Across panels A-C, Ybt-modified XAD demonstrated clear removal capabilities compared to controls. Repeated extractions with Ybt resulted in nearly 100% full solution removal (FIG. 33C). The preliminary data for iron and copper removal support the binding and removal potential of Ybt. As demonstrated in FIG. 33D, selective removal was observed for silver and nickel during initial and un-optimized preliminary assessment. The results were a promising basis for the refined and extended plans. For a more detailed study, the binding ability of Ybt and its analogs were thoroughly analyzed using ICP-MS. A mixture of Fe, Cu, Al, Zn, Pt, Au, Pd and, Ag were prepared each with a concentration of approximately 50 ppb. The mixtures were contacted with modified beads for 2 hours, and the metal contents were subsequently measured using ICP-MS. The difference in metal content before and after treatment has been adopted as a basis to calculate the percent of metal recovery (or removal).

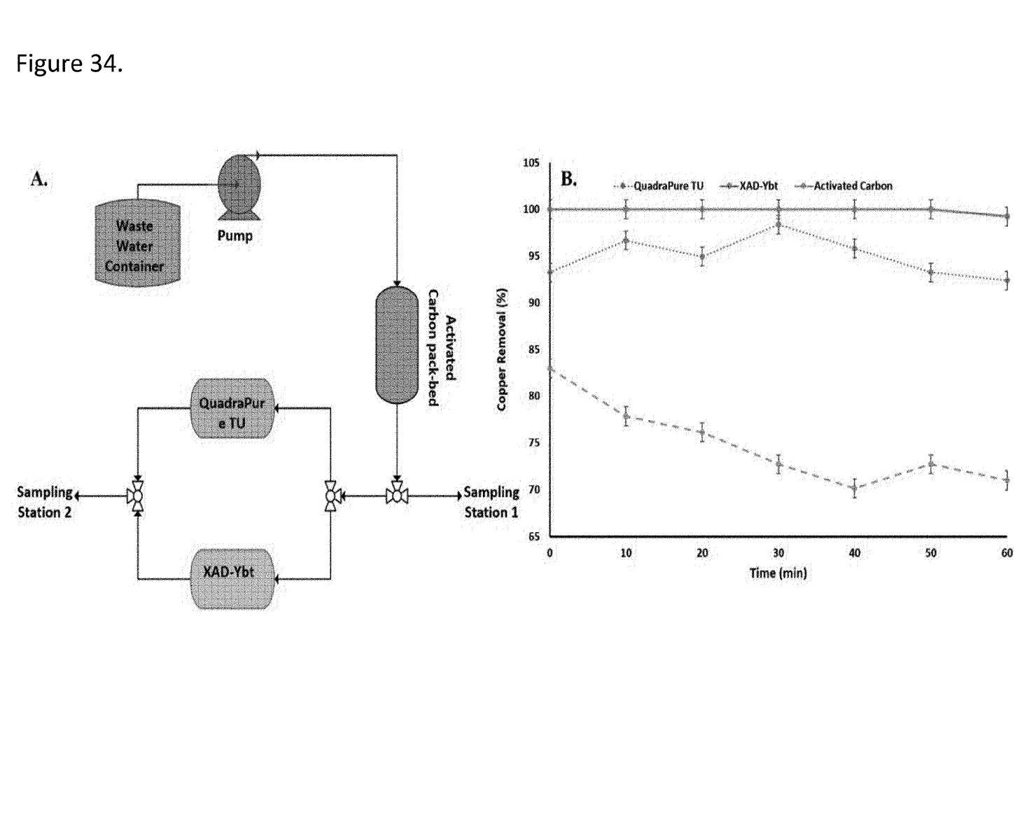

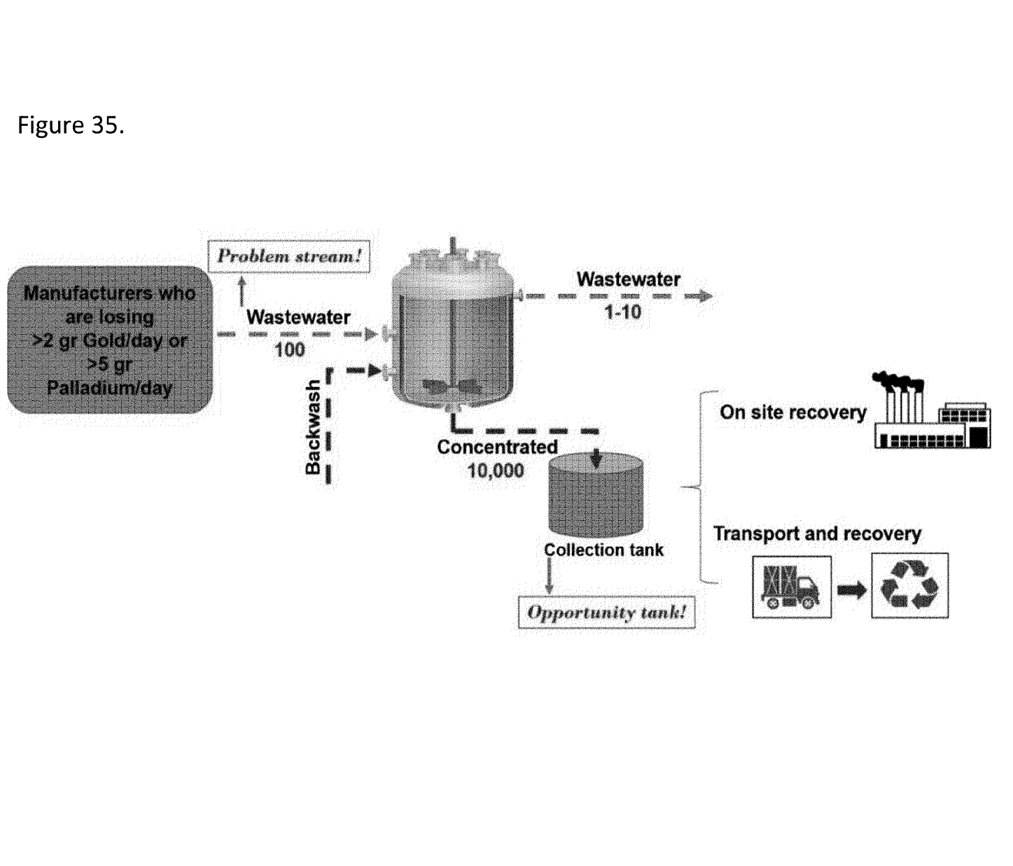

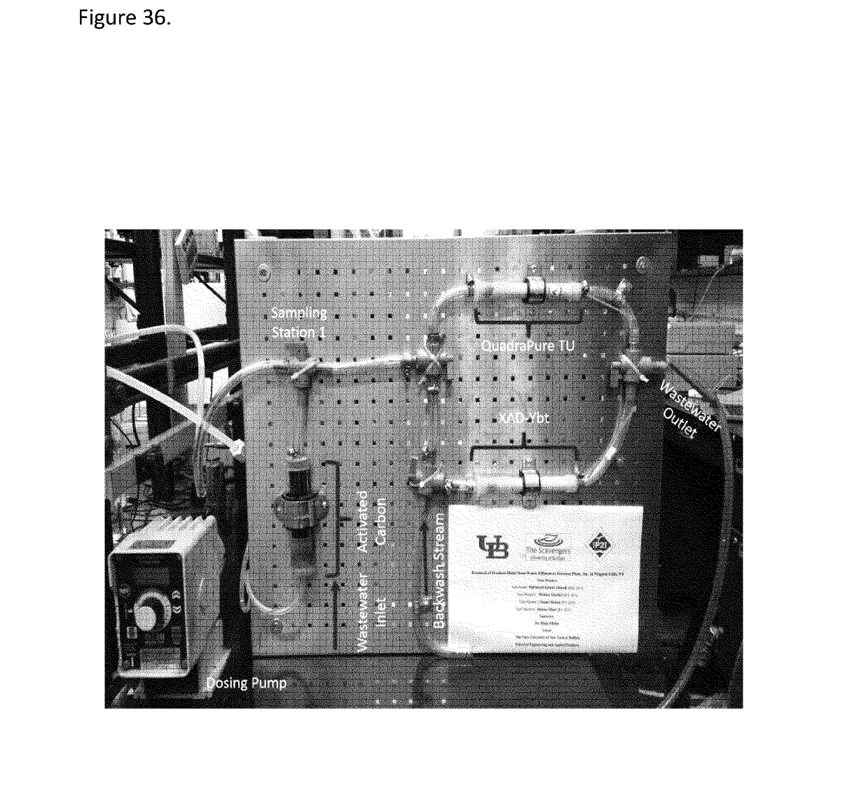

For a realistic applicable process, we have designed and implemented a continuous heavy metal removal and recovery system model. To be more specific, we have constructed a process model involving two sequential packed bed flow reactors with flow rate up to 2 lit/hour. Copper solutions with concentration between 1 to 10 ppm were treated as an effluent waste discharge model. The schematic is illustrated in FIG. 34A. Wastewater containing 3.3 ppm of copper was fed into a pack-bed containing 1) our developed beads, or 2) commercially available resins. The commercial resin utilized was QuadraPure TU from Johnson Matthey due to its ability to effectively binding to copper. FIG. 34B demonstrates that our developed XAD-Ybt beads offer elevated absorption kinetics compared to commercial scavenger. The commercial resin consistently removed approximately 25% of the copper. In comparison, our XAD-Ybt removed 90% of the copper for first 20 minutes; copper removal dropped to 50% for next 40 minutes, which indicated the resin had reached saturation. At the time 60 minutes, XAD-Ybt resins were backwashed with an altered pH. After backwash, data shows the resin had been regenerated and can retrieve their initial copper removal efficiency. Another option is to introduce a pretreatment column prior to the column loaded with scavengers or XAD-Ybt. This column was filled with activated carbon, a general, non-specific, metal remover. Activated carbon is known for its inexpensive but high ability to remove environmentally dangerous heavy metals. This module will not only remove hazardous heavy metals, it will also minimize the interfering effects that other base ions have on the function of the second reactor. FIG. 35B demonstrates this model can remove up to 100% of copper without any sign of saturation. Further measurements revealed that the activated carbon column by itself removed 75% of copper. For the next phase we are working on metal recovery by introducing a backwash unit (FIG. 36) with a pH gradient. FIG. 36 works as a simplified scheme for the plant treatment.

Experimental. We have proven the concept using a batch process. The XAD-Ybt and analog-XAD resin (40 mg) were added to small-scale (10 mL) samples of wastewater resulting from a variety of plating applications. Incubation occurred in a certain temperature, and after specific time points, beads were separated (by gravity settling) and washed with Millipore water twice. The metal content of the wastewater samples before and after treatment were measured using inductively coupled plasma mass spectrometry (ICP-MS). Due to the success of the laboratory model batch process, we assessed our findings to construct a small-scale continuous process model. The packed column reactor system was designed to purify 1 liter/hr of waste effluent. As previously described, the first column was packed with activated carbon, and the second column contained either commercial resins or our developed beads. The results show the model was able to remove up to 95% of copper from the wastewater.

After successful binding and removal of metals by Ybt, the loaded resin can then be integrated into a metal recovery and re-use plan. The first strategy to be pursued when recovering metal from the Ybt-metal-XAD beads will be to remove the Ybt-metal complex using methanol. This approach is based upon our previous research in which 90% of Ybt is removed from the XAD resin when they are washed with methanol. The metal could then be recovered from the Ybt via pH variation to eliminate chelation. Variation in pH can also be applied to Ybt-metal complexes while Ybt is still bound to the XAD bead to remove the recovered metal while re-generating the XAD-Ybt particle for future wastewater treatment. Each of these approaches can also be applied to different Ybt analog XAD beads. Once free metal has been recovered in solution, it will be recycled back to electroplating processes. If needed, purification steps (using pH-based separations) will be used to enrich certain metals for coating purposes. In this capacity, Ybt analogs that are selective for certain metals will be beneficial.

Economical Evaluation. A simplified environmental and economic analysis is presented here. A more sophisticated model and complete analysis follows. Table 4 lists the working parameters of daily waste effluent, including measured concentrations of four typical metals, and the total discharge volume. The reported concentrations come from actual discharged waste, meaning after the precipitation module. According to their claims, the concentration before entering the precipitation process can be up to 10 times more concentrated with metal ions.

The following calculations are based on the numbers from this table:

$$Au loss ( \frac{Kg}{year} ) = 70 \times {10}^{- 9} ( \frac{kg}{L} ) \times 40000 ( \frac{gallons}{day} ) \times 3.78 ( \frac{L}{gallons} ) \times 365 ( \frac{days}{year} ) = 3.86 \frac{Kg Au}{year}$$

$$Pd loss ( \frac{Kg}{year} ) = 70 \times {10}^{- 9} ( \frac{kg}{L} ) \times 40000 ( \frac{gallons}{day} ) \times 3.78 ( \frac{L}{gallons} ) \times 365 ( \frac{days}{year} ) = 0.39 \frac{Kg Pd}{year}$$

$$Ag loss ( \frac{Kg}{year} ) = 120 \times {10}^{- 9} ( \frac{kg}{L} ) \times 40000 ( \frac{gallons}{day} ) \times 3.78 ( \frac{L}{gallons} ) \times 365 ( \frac{days}{year} ) = 6.62 \frac{Kg Ag}{year}$$

$$Ni loss ( \frac{Kg}{year} ) = 636 \times {10}^{- 9} ( \frac{kg}{L} ) \times 40000 ( \frac{gallons}{day} ) \times 3.78 ( \frac{L}{gallons} ) \times 365 ( \frac{days}{year} ) = 20.55 \frac{Kg Ni}{year}$$

This brief list does not encompass all of the metal content in the waste effluent. The following calculation is based on the current price of each metal and the previous calculated numbers:

$$loss ( \$ ) = 3.86 ( \frac{Kg Au}{year} ) \times 36000 ( \frac{\$}{kg Au} ) + 0.39 ( \frac{Kg Pd}{year} ) \times 24000 ( \frac{\$}{kg Pd} ) + 6.62 ( \frac{Kg Ag}{year} ) \times 480 ( \frac{\$}{kg Ag} ) + 20.55 ( \frac{Kg Ni}{year} ) \times 20 ( \frac{\$}{kg Ni} ) \approx 152.00 \frac{K \$}{year}$$

TABLE 4
Measured parameters of waste effluent in Precious Plate.

| | Metal | | | |
|---|---|---|---|---|
| | Gold | Palladium | Silver | Nickel |
| concentration (PPM) | 0.07 | 0.007 | 0.12 | 0.636 |

| | |
|---|---|
| Discharge volume (gallons/day) | 40,000 |

Currently, there are no applications for recovering precious metals at such low concentrations (100 ppb) from wastewater.

For budgetary purposes assumptions were made to account for daily fluctuations in precious metal concentration and wastewater volume. An average volume of 10,000 liters of wastewater per day with a gold concentration of 50 ppb (0.05 mg/L) was used for calculating costs and returns. After determining the initial investment costs and operational costs associated with each application, it was clear that the feasibility of these products is highly dependent on both loading capacity and recovery efficiency of the resins. The loading capacity determines how much resin material is needed to remove a known concentration of metal, in this case 500 mg of gold per day.

$$Loading Capacity = \frac{x ( mg loaded metal )}{y ( g of resin )} \times 100 \\ Amount of resin needed per day ( g ) = \frac{500 mg gold}{Loading Capacity / 100}$$

Based on this calculation it can be seen that a higher loading capacity is desirable to reduce cost of resin. The average loading capacity of the metal scavenger and XAD-Ybt resin is taken to be 7% and 5%, respectively. It should also be noted that the loading capacity is more important for the metal scavenger because it cannot be recycled like the XAD resin. The recovery efficiency also weighs heavy on the cost outcome. A higher efficiency leads to more profit, and is therefore more advantageous. Based on the metal scavenger supplier's data, the recovery efficiency of metal scavengers is in the range of 1-15%, and the XAD-Ybt resin has a recovery efficiency of 60-80%. All calculations are based on these values.

Conclusions. We have demonstrated the improved metal reduction capabilities of our developed siderophore based resins in comparison to a commercial metal scavenger when applied to wastewater samples. These results support the use of this technology to address environmental concerns.

While the present disclosure has been particularly shown and described with reference to specific examples (some of which are preferred examples), it should be understood by those having skill in the art that various changes in form and detail may be made therein without departing from the spirit and scope of the present disclosure as disclosed herein.

1. A compound comprising the following structure: 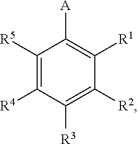
wherein R¹ is OH or NH₂,
R², R³, R⁴, and R⁵ are each independently H, OH, NH₂, SH, I, Br, Cl, F, C₁-C₆ alkyl, or one or more adjacent combinations of R², R³, R⁴, and/or R⁵ that together form one or more fused rings, and
A is selected from the group consisting of: 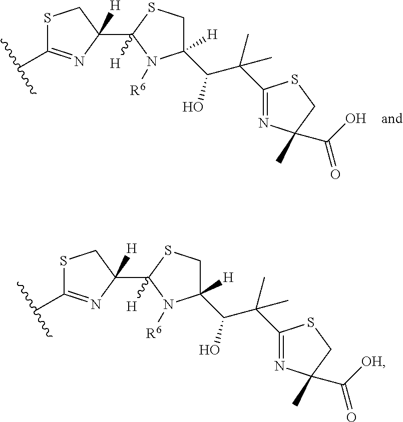
wherein R⁶ is H or CH₃.

2. The compound of claim 1, wherein R⁶ is H.

3. The compound of claim 1, wherein the compound is selected from the group consisting of:

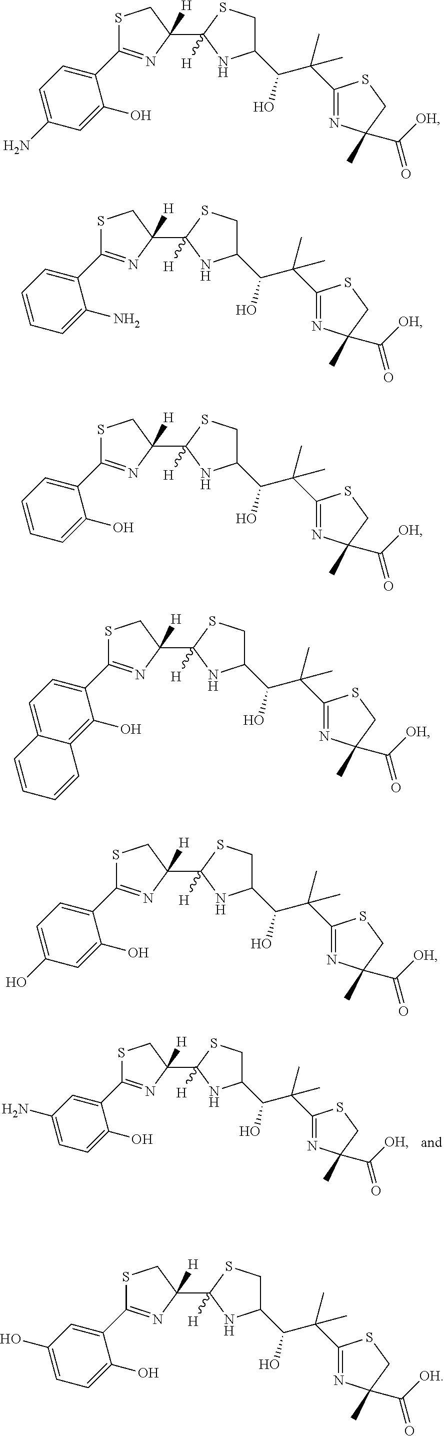

4. A method of making one or more compounds of claim 1, comprising:
contacting one or more microbes modified to produce the one or more compounds of claim 1 with a compound having the following structure: 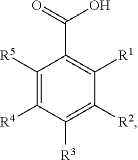
wherein R¹ is OH or NH₂, and R², R³, R⁴, and R⁵ are each independently H, OH, NH₂, SH, I, Br, Cl, F, C₁-C₆ alkyl, or one or more adjacent combinations of R², R³, R⁴, and/or R⁵ that together form one or more fused rings,
for a period of time sufficient to produce the one or more compounds of claim 1.

5. The method of claim 4, further comprising isolating the one or more compounds of claim 1.

6. A method of removing one or more metals from a metal-containing sample comprising:
a) contacting the metal-containing sample with one or more compounds of claim 1, wherein a complex between the one or more metals and the one or more compounds is formed;
b) isolating the complex of a) from the sample.

7. The method of claim 6, further comprising reversing the isolated complex between the one or more metals and the one or more compounds of claim 1 to provide one or more metal-free compounds of claim 1 and uncomplexed one or more metals.

8. The method of claim 7, further comprising repeating a) on the metal-containing sample or a second metal-containing sample using the metal-free compounds of claim 1 and isolating the complex between the one or more metals from the metal-containing sample and the metal-free compounds or the complex between the one or more metals of the second metal-containing sample and the metal-free compounds.

9. The method of claim 6, wherein the compounds of claim 1 are attached to a polymeric bead, polymeric resin, non-polymeric resin, activated carbon, or inorganic material.

10. The method of claim 9, wherein the polymeric bead comprises a non-ionic crosslinked polymer or a hydrophobic resin.

11. The method of claim 6, wherein the metal-containing sample is selected from the group consisting of aqueous samples, rust-containing surfaces, soil, electronic waste, and coal.

12. The method of claim 6, wherein the metal-containing sample is selected from the group consisting of water, tap water, pool water, wastewater, ocean water, freshwater, brine, jewelry, flatware, aquatic vessels, buildings, mine tailings/waste, semiconductor process effluent, fish farm growth environment, jewelry and plating effluents, solar panel process effluents, and wastewater treatment sludge or precipitants.

13. The method of claim 6, wherein the metal is selected from the group consisting of iron, copper, gold, silver, palladium, platinum, lithium, aluminum, bismuth, gallium, germanium, indium, rhodium, selenium, silicon, nickel, tellurium, zinc, manganese, arsenic, lead, mercury, cerium, scandium, yttrium, lanthanum, praseodymium, neodymium, promethium, samarium, europium, gadolinium, terbium, dysprosium, holmium, erbium, thulium, ytterbium, lutetium, and combinations thereof.

14. A composition comprising modified microbes comprising a compound of claim 1.

15. The composition of claim 14, wherein the modified microbes are modified Escherichia coli, Bacillus subtilis, Streptomyces coelicolor, Streptomyces lividans, Streptomyces venezuelae, Saccharomyces cerevisiae, Pichia pastoris, Aspergillus niger, Aspergillus nidulans, Pseudomonas aeruginosa, or a combination thereof.

16. The composition of claim 15, wherein the modified microbes are modified Escherichia coli.
